(19) United States
(12) Patent Application Publication
(10) Pub. No.: US 2026/0000307 A1
(43) Pub. Date: Jan. 1, 2026

(54) SYSTEMS AND METHODS FOR RAPID BLOOD PRESSURE MONITORING

(71) Applicant: Lukasz R. Kiljanek, Chesapeake Beach, MD (US)

(72) Inventor: Lukasz R. Kiljanek, Chesapeake Beach, MD (US)

(21) Appl. No.: 19/256,120

(22) Filed: Jun. 30, 2025

Related U.S. Application Data
(60) Provisional application No. 63/666,125, filed on Jun. 29, 2024.
(60) Provisional application No. 63/784,037, filed on Apr. 5, 2025.
(60) Provisional application No. 63/795,372, filed on Apr. 26, 2025.
(60) Provisional application No. 63/795,505, filed on Apr. 27, 2025.
(60) Provisional application No. 63/810,081, filed on May 22, 2025.

(51) Int. Cl. A61B 5/022; A61B 5/0235

(52) U.S. Cl. CPC A61B 5/02208; A61B 5/02233; A61B 5/0235; A61B 2560/0223

(57) ABSTRACT

Systems and methods are disclosed for blood pressure analysis. In some examples, a system receives sensor data from a sensor during a time period to monitor a blood pressure wave signal in the sensor data. The system inflates a cuff (worn by a patient) with a gas until a characteristic of the pulse wave signal crosses a characteristic threshold from a first side to a second side at a first time within the time period. The cuff is at a first pressure at the first time. The system deflates the cuff until the characteristic of the pulse wave signal crosses the characteristic threshold from the second side to the first side at a second time within the time period. The cuff is at a second pressure at the second time. The system identifies a blood pressure value corresponding to the patient as being between the first pressure and the second pressure.

### CROSS-REFERENCE TO RELATED APPLICATIONS

The present application claims the priority benefit of U.S. provisional application No. 63/666,125 filed Jun. 29, 2024 and entitled “New faster, more accurate and precise blood pressure meter with novel business model and phantom to validate blood pressure monitors,” U.S. provisional application No. 63/784,037 filed Apr. 5, 2025 and entitled “Blood pressure monitor 2.0,” U.S. provisional application No. 63/795,372 filed Apr. 26, 2025 and entitled “New generation blood pressure monitor. Blood pressure monitor 2.0,” U.S. provisional application No. 63/795,505 filed Apr. 27, 2025 and entitled “BP monitor 2.0,” and U.S. provisional application No. 63/810,081 filed May 22, 2025 and entitled “improved BP monitor,” the disclosures of which are hereby incorporated by reference in their entireties.

### TECHNICAL FIELD

This disclosure relates to physiological attribute monitoring systems and methods, and in particular, to physiological attribute monitoring systems and methods that controls cuff inflation and/or sensor analysis to improve speed, accuracy, reliability, and flexibility of determining physiological attributes such as diastolic blood pressure, systolic blood pressure, central venous pressure, heart rate, respiratory rate, and/or cardiac output.

### BACKGROUND

Blood pressure monitors are medical devices used to measure the force of blood against the walls of the arteries as the heart pumps. Blood pressure monitors can help detect and manage conditions such as hypertension, a major risk factor for heart disease and stroke. It generally takes multiple minutes to determine diastolic blood pressure and systolic blood pressure for a patient using a traditional blood pressure monitor.

While a traditional blood pressure monitor is measuring a patient's blood pressure, the patient's blood flow is at least partially cut off, slowed, or even stopped, which can be detrimental to the patient's health, especially if the patient already has cardiovascular issues such as atrial fibrillation, arrhythmia, heart disease, or aortic disease. For certain situations, such as a patient undergoing dialysis or serious surgery in an intensive care unit (ICU) (e.g., with high risk of significant blood loss), it is desirable for doctors to monitor blood pressure regularly-every 15 minutes in some situations, or even every minute in others. However, this puts patients that are already suffering under more cardiovascular stress, potentially lowering a patient's probability of surviving a serious surgery, or hindering the patient's recovery.

Additionally, traditional blood pressure monitors are often inaccurate because they do not properly account for different sizes of different patients. Traditional blood pressure monitors offer only a handful of different cuff sizes for different patient sizes (e.g., different patient arm circumferences). However, especially for patients that are between one size and another, or at the edge of a particular size range, it can be unclear which size of cuff to use, and neither may be fully accurate due to the stepwise changes between the two cuff sizes. Sometimes, the wrong cuff size is used, either due to lack of resources (e.g., physicians in remote areas might only have access to one cuff size) or lack of training. Using a cuff that is too small generally results in overestimation of blood pressure, potentially leading to a misdiagnosis of hypertension and/or overtreatment with medication, or even unnecessary surgery. Using a cuff that is too large generally results in underestimation of blood pressure, potentially causing a delay in diagnosis and/or treatment of hypertension. Studies have shown that changing from one cuff size to the next can cause a change in blood pressure measurement of approximately 4.8 millimeters of mercury (mmHg). Studies have shown that changing two cuff sizes can cause a change in blood pressure measurement of approximately 19.5 mmHg. These are significant deviations that can make a significant difference between a diagnosis of a patient as healthy or as severely sick.

### SUMMARY

Systems and methods are disclosed for blood pressure analysis for a patient. In some examples, a system receives sensor data from a sensor during a time period to monitor a characteristic of a blood pressure wave signal in the sensor data during the time period. A cuff is worn by a patient during the time period. The system inflates at least one bladder of the cuff with a gas until the characteristic of the blood pressure wave signal crosses a characteristic threshold from a first side of the characteristic threshold to a second side of the characteristic threshold at a first time within the time period. The at least one bladder is at a first pressure at the first time. The system deflates the at least one bladder of the cuff until the characteristic of the blood pressure wave signal crosses the characteristic threshold from the second side of the characteristic threshold to the first side of the characteristic threshold at a second time within the time period. The at least one bladder is at a second pressure at the second time. The system identifies a blood pressure value corresponding to the patient as being between the first pressure and the second pressure.

In one example, an apparatus is provided that includes one or more memories and one or more processors coupled to the one or more memories. The at least one processor is configured to perform operations. The operations include receiving sensor data from a sensor during a time period to monitor a characteristic of a blood pressure wave signal in the sensor data during the time period. a cuff is worn by the patient during the time period. The operations include inflating at least one bladder of the cuff with a gas until the characteristic of the blood pressure wave signal crosses a characteristic threshold from a first side of the characteristic threshold to a second side of the characteristic threshold at a first time within the time period. The at least one bladder is at a first pressure at the first time. The operations include deflating the at least one bladder of the cuff until the characteristic of the blood pressure wave signal crosses the characteristic threshold from the second side of the characteristic threshold to the first side of the characteristic threshold at a second time within the time period. The at least one bladder is at a second pressure at the second time. The operations include identifying a blood pressure value corresponding to the patient, wherein the blood pressure value is between the first pressure and the second pressure.

In another example, a method is provided. The method includes receiving sensor data from a sensor during a time period to monitor a characteristic of a blood pressure wave signal in the sensor data during the time period. a cuff is worn by the patient during the time period. The method includes inflating at least one bladder of the cuff with a gas until the characteristic of the blood pressure wave signal crosses a characteristic threshold from a first side of the characteristic threshold to a second side of the characteristic threshold at a first time within the time period. The at least one bladder is at a first pressure at the first time. The method includes deflating the at least one bladder of the cuff until the characteristic of the blood pressure wave signal crosses the characteristic threshold from the second side of the characteristic threshold to the first side of the characteristic threshold at a second time within the time period. The at least one bladder is at a second pressure at the second time. The method includes identifying a blood pressure value corresponding to the patient, wherein the blood pressure value is between the first pressure and the second pressure.

In another example, a non-transitory computer-readable medium is provided that has stored thereon instructions that, when executed by one or more processors, cause the one or more processors to perform operations. The operations include receiving sensor data from a sensor during a time period to monitor a characteristic of a blood pressure wave signal in the sensor data during the time period. a cuff is worn by the patient during the time period. The operations include inflating at least one bladder of the cuff with a gas until the characteristic of the blood pressure wave signal crosses a characteristic threshold from a first side of the characteristic threshold to a second side of the characteristic threshold at a first time within the time period. The at least one bladder is at a first pressure at the first time. The operations include deflating the at least one bladder of the cuff until the characteristic of the blood pressure wave signal crosses the characteristic threshold from the second side of the characteristic threshold to the first side of the characteristic threshold at a second time within the time period. The at least one bladder is at a second pressure at the second time. The operations include identifying a blood pressure value corresponding to the patient, wherein the blood pressure value is between the first pressure and the second pressure.

In another example, an apparatus is provided. The apparatus includes means for receiving sensor data from a sensor during a time period to monitor a characteristic of a blood pressure wave signal in the sensor data during the time period. a cuff is worn by the patient during the time period. The apparatus includes means for inflating at least one bladder of the cuff with a gas until the characteristic of the blood pressure wave signal crosses a characteristic threshold from a first side of the characteristic threshold to a second side of the characteristic threshold at a first time within the time period. The at least one bladder is at a first pressure at the first time. The apparatus includes means for deflating the at least one bladder of the cuff until the characteristic of the blood pressure wave signal crosses the characteristic threshold from the second side of the characteristic threshold to the first side of the characteristic threshold at a second time within the time period. The at least one bladder is at a second pressure at the second time. The apparatus includes means for identifying a blood pressure value corresponding to the patient, wherein the blood pressure value is between the first pressure and the second pressure.

This summary is not intended to identify key or essential features of the claimed subject matter, nor is it intended to be used in isolation to determine the scope of the claimed subject matter.

### BRIEF DESCRIPTION OF THE DRAWINGS

Illustrative embodiments of the present application are described in detail below with reference to the following figures:

FIG. 1 is a block diagram illustrating an architecture of a physiological attribute monitoring system, in accordance with some examples;

FIG. 2 is a block diagram illustrating use of a physiological attribute monitoring system that determines systolic blood pressure and diastolic blood pressure during deflation of a cuff that is previously inflated using a pump, in accordance with some examples;

FIG. 3 is a block diagram illustrating use of a physiological attribute monitoring system that determines systolic blood pressure and/or diastolic blood pressure based on convergence upon threshold(s) associated with these blood pressure values, and with a cuff that is inflated using a compressed gas canister, in accordance with some examples;

FIG. 4 is a graph diagram illustrating changes to cuff pressure in a process involving sequential inflations and deflations of a cuff of a physiological attribute monitoring system to converge on a cuff pressure value indicative of a blood pressure value (e.g., systolic blood pressure or diastolic blood pressure) of a patient, in accordance with some examples;

FIG. 5 is a block diagram illustrating a physiological attribute monitoring system with a cuff around a body part of a patient, where the cuff is divided (along a length of the body part) into multiple gas bladders that can be inflated and/or deflated independently, and where the gas bladders are further divided (along the circumference of the body part) into multiple gas pillows, in accordance with some examples;

FIG. 6 is a block diagram illustrating a physiological attribute monitoring system with a cuff around a body part of a patient, where the cuff is divided (along a length of the body part) into multiple gas bladders that can be inflated and/or deflated independently, in accordance with some examples;

FIG. 7 is a graph diagram illustrating a comparison between blood pressure values measured using two different blood pressure monitor systems, or two different sensors (associated with different cuffs or gas bladders) of a single blood pressure monitor system, in accordance with some examples;

FIG. 8 is a block diagram illustrating a physiological attribute monitoring system with a cuff that is divided (along the circumference of the body part) into multiple gas pillows, in accordance with some examples;

FIG. 9 is a block diagram illustrating a physiological attribute monitoring system with a cuff that includes fasteners along a surface of the cuff, in accordance with some examples;

FIG. 10 is a block diagram illustrating an example of an artificial intelligence system for training, use of, and/or updating of one or more machine learning model(s) that are used to generate physiological attribute curve(s), physiological attribute value(s), and/or recommendation(s), in accordance with some examples;

FIG. 11 is a block diagram illustrating a RAG system that may be used to implement some aspects of the technology, in accordance with some examples.

FIG. 12 is a conceptual diagram illustrating a process for dynamically updating an output (that is generated using ML model(s)) in a continuous fashion as further data continues to be received over time, in accordance with some examples.

FIG. 13 is a flow diagram illustrating a process for physiological attribute analysis for a patient, in accordance with some examples; and

FIG. 14 is a block diagram of an exemplary computing device that may be used to implement some aspects of the technology.

### DETAILED DESCRIPTION

It generally takes multiple minutes to determine diastolic blood pressure and systolic blood pressure for a patient using a traditional blood pressure monitor. While a traditional blood pressure monitor is measuring a patient's blood pressure, the patient's blood flow is at least partially cut off, slowed, or even stopped, which can be detrimental to the patient's health, especially if the patient already has cardiovascular issues such as atrial fibrillation, arrhythmia, heart disease, or aortic disease. For certain situations, such as a patient undergoing dialysis or serious surgery in an intensive care unit (ICU) (e.g., with high risk of significant blood loss), it is desirable for doctors to monitor blood pressure regularly-every 15 minutes in some situations, or even every minute in others. However, this puts patients that are already suffering under more cardiovascular stress, potentially lowering a patient's probability of surviving a serious surgery, or hindering the patient's recovery.

Systems and methods are described herein for improved blood pressure monitoring that provides faster blood pressure readings (e.g., in a matter of seconds) and more accurate blood pressure readings. Thus, even for situations where a patient undergoes periodic blood pressure testing (e.g., every 15 minutes or even every minute), effects on the patient's blood flow (e.g., blood flow being at least partially cut off, slowed, or even stopped) are minimized due to the significantly faster blood pressure measurement.

Additionally, traditional blood pressure monitors are often inaccurate because they do not properly account for different sizes of different patients. Traditional blood pressure monitors offer only a handful of different cuff sizes for different patient sizes (e.g., different patient arm circumferences). However, especially for patients that are between one size and another, or at the edge of a particular size range, it can be unclear which size of cuff to use, and neither may be fully accurate due to the stepwise changes between the two cuff sizes. Sometimes, the wrong cuff size is used, either due to lack of resources (e.g., physicians in remote areas might only have access to one cuff size) or lack of training. Using a cuff that is too small generally results in overestimation of blood pressure, potentially leading to a misdiagnosis of hypertension and/or overtreatment with medication, or even unnecessary surgery. Using a cuff that is too large generally results in underestimation of blood pressure, potentially causing a delay in diagnosis and/or treatment of hypertension. Studies have shown that changing from one cuff size to the next can cause a change in blood pressure measurement of approximately 4.8 millimeters of mercury (mmHg). Studies have shown that changing two cuff sizes can cause a change in blood pressure measurement of approximately 19.5 mmHg. These are both significant deviations that can make a significant difference between a diagnosis of a patient as healthy or as sick, potentially leading to overmedication, undermedication, or misdiagnosis.

Systems and methods are disclosed for blood pressure analysis for a patient. In some examples, a system receives sensor data from a sensor during a time period to monitor a characteristic of a blood pressure wave signal in the sensor data during the time period. A cuff is worn by a patient during the time period. The system inflates at least one bladder of the cuff with a gas until the characteristic of the blood pressure wave signal crosses a characteristic threshold (e.g., amplitude crosses an amplitude threshold, slope crosses a slope threshold), from a first side of the threshold to a second side of the threshold at a first time within the time period. The at least one bladder is at a first pressure at the first time. The system deflates the at least one bladder of the cuff until the characteristic of the blood pressure wave signal crosses the threshold from the second side of the threshold to the first side of the threshold at a second time within the time period. The at least one bladder is at a second pressure at the second time. The system identifies a blood pressure value corresponding to the patient as being between the first pressure and the second pressure.

The systems and methods disclosed herein solve a number of technical problems, and provide a number of technical improvements. For instance, the systems and methods described herein can improve the speed, efficiency, accuracy, flexibility, adaptability, and reliability of blood pressure monitoring.

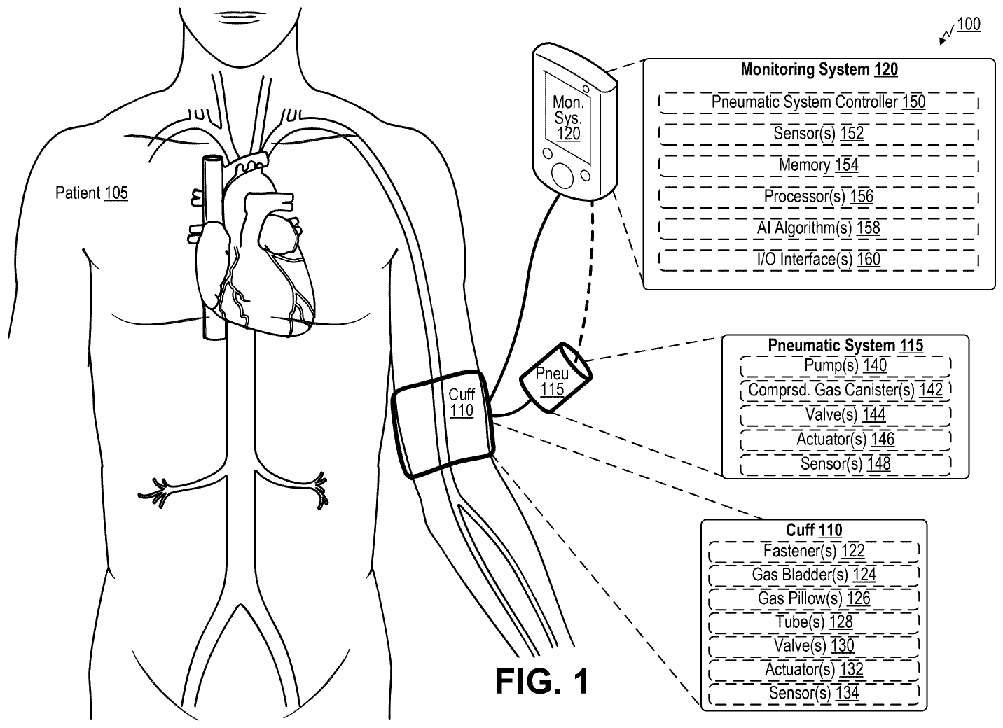

FIG. 1 is a block diagram illustrating an architecture of a physiological attribute monitoring system 100. The physiological attribute monitoring system 100 includes a cuff 110 that is worn by a patient 105. The physiological attribute monitoring system 100 includes a pneumatic system 115 that inflates and/or deflates the cuff 110 while the patient 105 is wearing the cuff 110. The physiological attribute monitoring system 100 includes a monitoring system 120 that analyzes pressure data, audio data, and/or other sensor data to identify blood pressure wave signals at different cuff pressures, and to determine blood pressure values (e.g., diastolic blood pressure, systolic blood pressure, and/or central venous pressure) based on the blood pressure wave signals at different cuff pressures and other bodily functions, signals and/or waveforms, such as, heart rate, respiratory rate, and/or cardiac output, based on various sensors.

The cuff 110 is designed to be wrapped around the arm, thigh, or another body part of the patient 105, for instance as illustrated in FIG. 1. The cuff 110 is responsible for applying pressure to the arm, which is essential for measuring blood pressure. Fasteners 122 secure the cuff in place, ensuring it remains properly positioned during the measurement process. The fasteners 122 can include hook and loop fasteners (e.g. Velcro® fasteners), magnetic fasteners (e.g., magnets, ferromagnetic materials, ferromagnets, electromagnets that can fasten and/or release in controlled manner, metals, rare earth minerals, or combinations thereof), clips, pins, hooks, loops, screws, threads, elastics, ties, adhesives (e.g., glues, tapes, and/or reusable adhesives), or combinations thereof.

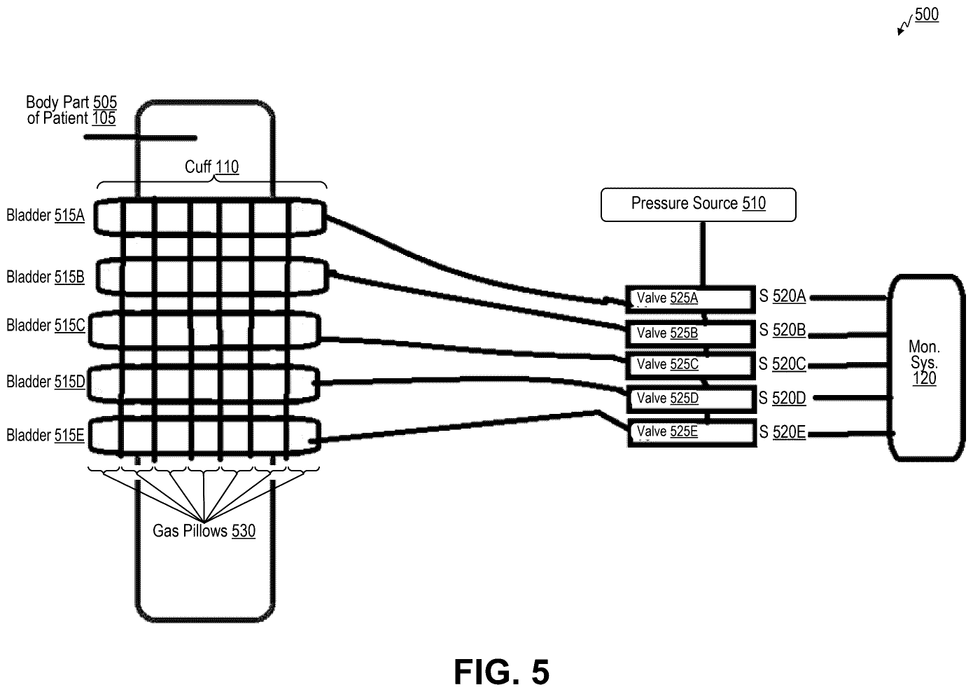

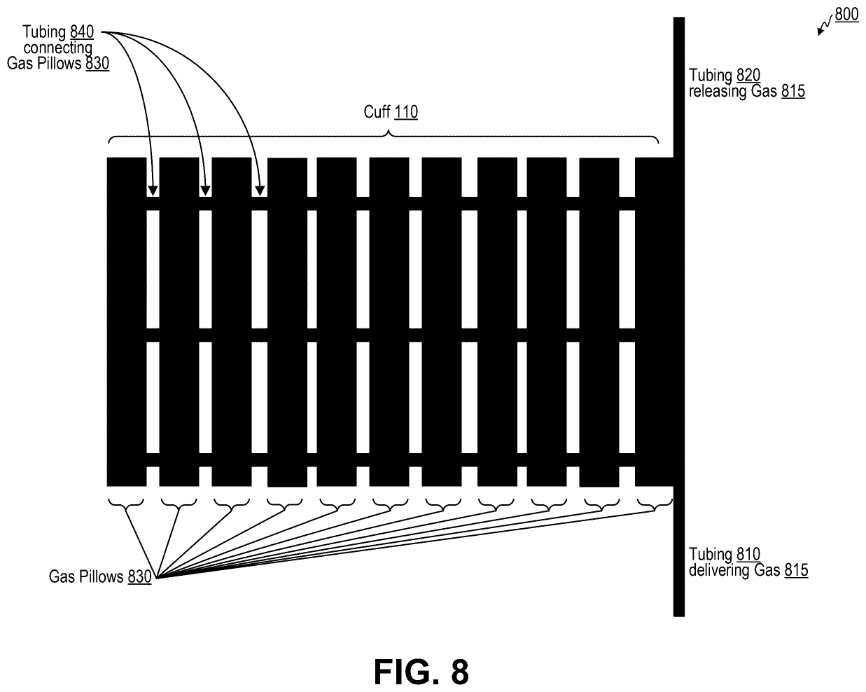

Within the cuff, one or more gas bladder(s) 124 and/or gas pillow(s) 126 are inflated and deflated by the pneumatic system 115 to apply pressure to the patient 105 and release pressure from the patient 105, respectively. In some examples, the gas bladder(s) 124 and/or gas pillow(s) 126 are coupled to one another, and/or to the pneumatic system 115, via tube(s) 128. In some examples, the gas bladder(s) 124 are further sub-divided into gas pillow(s) 126 that are connected to one another via tube(s) 128. The tubes 128 connect various components, facilitating the flow of gas to and from the gas bladders and pillows, enabling the inflation and deflation processes. In some examples, the gas pillow(s) 126 provide additional cushioning and support, distributing pressure evenly for enhanced comfort and/or accuracy. Examples of gas pillow(s) 126 of a cuff 110 (coupled to one another with tube(s) 128) are illustrated in FIG. 5 and FIG. 8. For instance, the different gas pillows 830 of FIG. 8 is an example of the tube(s) 128.

In some aspects, the gas bladder(s) 124 can be referred to as gas pillow(s), as bladders, as pillows, or as portion(s) of the cuff 110. In some examples, the gas pillow(s) 126 can be referred to as gas bladder(s), as pillows, as bladders, and/or as portion(s) of the cuff 110.

In some examples, the cuff 110 includes valve(s) 130 that control flow of gases from the pneumatic system 115, to the pneumatic system 115, throughout the 110 (e.g., between gas bladder(s) 124 and/or gas pillow(s) 126), or a combination thereof. In some examples, the cuff 110 includes actuator(s) 132 (e.g., motors, linear actuators) that can be actuated (e.g., activated or deactivated) by the pneumatic system 115 and/or the monitoring system 120, for instance to open or close the valve(s) 130, to move a portion of the cuff 110 relative to another portion of the cuff 110, to move a portion of the cuff 110 relative to the patient 105, or a combination thereof. The valve(s) 130 can control the flow of gas, opening and closing to regulate the pressure applied by the cuff 110. The actuator(s) 132 can operate these valves, receiving signals from the pneumatic system 115 and/or the monitoring system 120 to manage gas flow and pressure.

In some examples, the cuff 110 includes sensor(s) 134. In some examples, the pneumatic system 115 includes sensor(s) 148. In some examples, the monitoring system 120 includes sensor(s) 152. These various sensors of the system can capture sensor data to monitor the pressure of the cuff, the blood pressure of the patient 105, audio signals indicative of the blood flow of the patient 105 (e.g., Korotkoff sounds), oscillations caused by blood flow, blood volume changes, pulse oximetry changes, and/or other indications of blood pressure. The sensor(s) 152 can include sensor(s) that are built in the monitoring system 120 (e.g., broad angle view camera sensor, providing data for analysis of posture therefore for determination whether patient is at rest, before or during the operation of the system). The sensors(s) 134 can include sensor(s) that are part of the cuff 110 or included in the cuff 110, such as accelerometers, microphones, pressure sensors, or other types of sensors discussed herein, for instance providing data for analysis of posture therefore for determination whether patient is being at rest, before or during the operation of the system, and also providing data for the AI algorithm(s) 158 (e.g., ML Engine 1020) for AI analysis, to be able to better discriminate from movement artifacts. The sensor data can be received from the sensor(s) 134, the sensor(s) 148, and/or the sensor(s) 152 by the monitoring system 120. The sensor data from the sensor(s) 134, the sensor(s) 148, and/or the sensor(s) 152 can be analyzed by the monitoring system 120 to determine blood pressure values, such as the diastolic blood pressure of the patient 105 and/or the systolic blood pressure of the patient 105, based on the sensor data.

The sensors (e.g., the sensor(s) 134, the sensor(s) 148, and/or the sensor(s) 152) can include pressure sensors, such as Micro-Electro-Mechanical Systems (MEMS) pressure sensors, piezoresistive sensors, capacitive, piezoelectric, Micro-Optome-Mechanical Systems (MOMS), absolute pressure sensors. The pressure sensors can be used to determine the pressure of the cuff 110 (e.g., of gas bladder(s) 124 and/or gas pillow(s) 126 of the cuff 110). The pressure sensors can be used to detect pressure oscillations caused by blood flow.

The sensors (e.g., the sensor(s) 134, the sensor(s) 148, and/or the sensor(s) 152) can include microphones or other audio sensors, such as condenser microphones, electret microphones, or MEMS microphones. The physiological attribute monitoring system 100 can use microphones to perform auscultatory detection (e.g., listening for the Korotkoff sounds that occur as the cuff inflates and/or deflates and blood flows through the brachial artery or any artery). The physiological attribute monitoring system 100 can use microphones to enhance accuracy of pressure-based blood pressure monitoring by listening for Korotkoff sounds alongside oscillation detection. In some examples, specific microphones can be chosen that are capable of withstanding the relatively high static pressures exerted by the inflating cuff, that are sensitive to low frequency ranges (e.g., the Korotkoff sounds are low-frequency signals), and/or that function consistently despite temperature changes (e.g., caused by pressure changes and/or bodily proximity). In some examples, any filters (e.g., high-pass filters, band-pass filters harmonic filters, narrow spectra filters, other types of filters, or a combination thereof) can improve sensitivity of the microphones to the low frequency ranges that Korotkoff sounds reside in, and/or can help filter out irrelevant noise. In some examples preprocessing using AI or ML for any signal recorded from any sensors can be performed. In some examples, temperature compensation circuitry (e.g., coolers, heaters, heat sinks, fans, and the like) can be used to compensate for temperature changes.

The sensors (e.g., the sensor(s) 134, the sensor(s) 148, and/or the sensor(s) 152) can include optical sensors, such as cameras, photoplethysmography (PPG) sensors (e.g., which measure blood volume changes by shining light onto the skin and detecting the amount of light reflected or transmitted), and/or pulse oximetry sensors.

The sensors (e.g., the sensor(s) 134, the sensor(s) 148, and/or the sensor(s) 152) can include bioimpedance sensors (e.g., measure the electrical impedance of body tissues, which can be related to blood volume and flow, and thus blood pressure), mechanical sensors such as ballistocardiography (BCG) sensors and/or seismocardiography (SCG) sensors (e.g., to detect small vibrations and movements caused by the heart and blood flow), or combinations thereof.

The sensors (e.g., the sensor(s) 134, the sensor(s) 148, and/or the sensor(s) 152) can include other types of sensors, such as thermometers, accelerometers, gyrometers, inertial measurement units (IMU), biometric sensors, and/or other sensors. In some examples, such sensors can be used by the monitoring system 120 to detect, and accommodate for, changes in temperature, position, orientation, and/or movement, all of which can in some examples affect blood pressure readings in certain situations

The pneumatic system 115, which includes pumps and compressed gas canisters, supplies the gas needed to inflate the cuff 110, generating and maintaining the required pressure for accurate measurements. In some examples, the pneumatic system 115 includes pump(s) 140 (e.g., manual pump(s) or actuated pump(s)) that the pneumatic system 115 can use to inflate the cuff 110 (e.g., to inflate the gas bladder(s) 124 and/or the gas pillow(s) 126 of the cuff 110). The system can also be connected to central gas distribution systems, for example like the ones present in ICU and in other places (e.g., oxygen line), to provide high pressure high flow gas instantaneously for even more efficient inflation of the gas pillow(s) 126, gas bladder(s) 124, and/or cuff 110.

In some examples, the pneumatic system 115 includes, and/or receives, compressed gas canister(s) 142 (e.g., with compressed air, hydrogen, helium, nitrogen, oxygen, carbon dioxide, argon, nitrous oxide, another gas, or a combination thereof) that the pneumatic system 115 can use to inflate the cuff 110 (e.g., to inflate the gas bladder(s) 124 and/or the gas pillow(s) 126 of the cuff 110). The pneumatic system 115 can include valve(s) 144 that control flow of gases from the pneumatic system 115 (e.g., from the compressed gas canister(s) 142 and/or the pump(s) 140) to the cuff 110 or vice versa. In some examples, the pneumatic system 115 includes actuator(s) 146 (e.g., motors, linear actuators) that can be actuated (e.g., activated or deactivated) by the pneumatic system 115 and/or the monitoring system 120, for instance to open or close the valve(s) 144, to supply gas to the cuff 110 or to stop supplying gas to the cuff 110. The valve(s) 144 can control the flow of gas, opening and closing to regulate the pressure applied by the cuff 110. The actuator(s) 146 can operate these valves, receiving signals from the pneumatic system 115 and/or the monitoring system 120 to manage gas flow and pressure.

In some examples, the pressure in the gas pillow(s) 126, the gas bladder(s) 124, and/or the cuff 110 can also be established, during deflation, using actuator(s) (e.g., actuator(s) 132, actuator(s) 146) and/or valves (e.g., valve(s) 130, valve(s) 144) to deflate these gas pillow(s) 126, the gas bladder(s) 124, and/or the cuff 110 to set or desired pressures. In some examples, to achieve more fine and granular pressure control in gas pillow(s) 126, the gas bladder(s) 124, and/or the cuff 110, the actuator(s) and/or valve(s) decrease the inflow or outflow rates according to how far the current pressure in gas pillow(s) 126, the gas bladder(s) 124, and/or the cuff 110 is from desired pressure in gas pillow(s) 126, the gas bladder(s) 124, and/or the cuff 110. So for example, if the pressure in the gas pillow(s) 126, the gas bladder(s) 124, and/or the cuff 110 is much lower than desired, the inflation will start with high flow rate, but as the pressure in gas pillow(s) 126, the gas bladder(s) 124, and/or the cuff 110 approaches the desired pressure for that gas pillow(s) 126, the gas bladder(s) 124, and/or the cuff 110 (which can be known to the physiological attribute monitoring system 100 based on its software and algorithms, and/or based on attributes of the patient 105), the flow rate decreases gradually to zero. For example, if the pressure in the gas pillow(s) 126, the gas bladder(s) 124, and/or the cuff 110 is much higher than desired, the deflation starts with high flow rate-but as the pressure in gas pillow(s) 126, the gas bladder(s) 124, and/or the cuff 110 approaches the desired pressure for that gas pillow(s) 126, the gas bladder(s) 124, and/or the cuff 110 (which can be known to the system based on its software and algorithms, and/or based on attribute(s) of the patient 105) the flow rate decreases gradually to zero. If the pressure in any gas pillow(s) 126, the gas bladder(s) 124, and/or the cuff 110, is different than what is desired by the physiological attribute monitoring system 100 at any point, for its optimal operations (which can be known to the system based on its software and algorithms, and/or based on attribute(s) of the patient 105) the physiological attribute monitoring system 100 can automatically increase or decrease the pressure in gas pillow(s) 126, the gas bladder(s) 124, and/or the cuff 110.

The monitoring system 120 encompasses sensors, processors, algorithms, and/or interfaces that are used together to analyze sensor data to determine blood pressure values (e.g., diastolic blood pressure, systolic blood pressure, and/or central venous pressure) and other physiologic or bodily measurements and signals in many embodiments (i.e. heart rate, cardiac output, arterial blood hemoglobin oxygen saturation), for the patient 105. The monitoring system 120 includes a pneumatic system controller 150 that can manage the pneumatic system 115, for instance controlling when the pneumatic system 115 supplies gas to the cuff 110 to inflate the cuff, when the pneumatic system 115 opens valve(s) in the cuff 110 to deflate the cuff (or when the pneumatic system controller 150 itself opens valve(s) in the cuff 110 to deflate the cuff), and/or otherwise controlling the flow and pressure of gas within the physiological attribute monitoring system 100. The sensor(s) 152 can include any of the types of sensors described herein, and the monitoring system 120 can use the sensor(s) 152 to detect, measure, and/or monitor cuff pressure, Korotkoff audio, oscillations, physiological parameters related to blood pressure, and/or other indicators of blood pressure. In some examples, portions of certain sensor(s) are located within the pneumatic system 115 and/or the monitoring system 120 (e.g., portions that receive and/or interpret electrical signals from probes), while other portions of the sensor(s) (e.g., probes) are located within, coupled to, or near the cuff 110 or the patient 105.

The monitoring system 120 can include a memory 154, which can include random access memory (RAM), read-only memory (ROM), at least one non-transitory computer readable storage medium, a portable or removable storage medium, or a combination thereof. The memory 154 can store instructions for computer programs executed by processor(s) 156 of the monitoring system 120, can store sensor data from the sensor(s) of the physiological attribute monitoring system 100 (e.g., the sensor(s) 134, the sensor(s) 148, and/or the sensor(s) 152), can store model data (e.g., parameters, hyperparameters, fine-tuning data) corresponding to one or more machine learning (ML) model(s) and/or other artificial intelligence algorithm(s) 158, or combinations thereof. The monitoring system 120 can include one or more artificial intelligence algorithm(s) 158, such as one or more trained machine learning models.

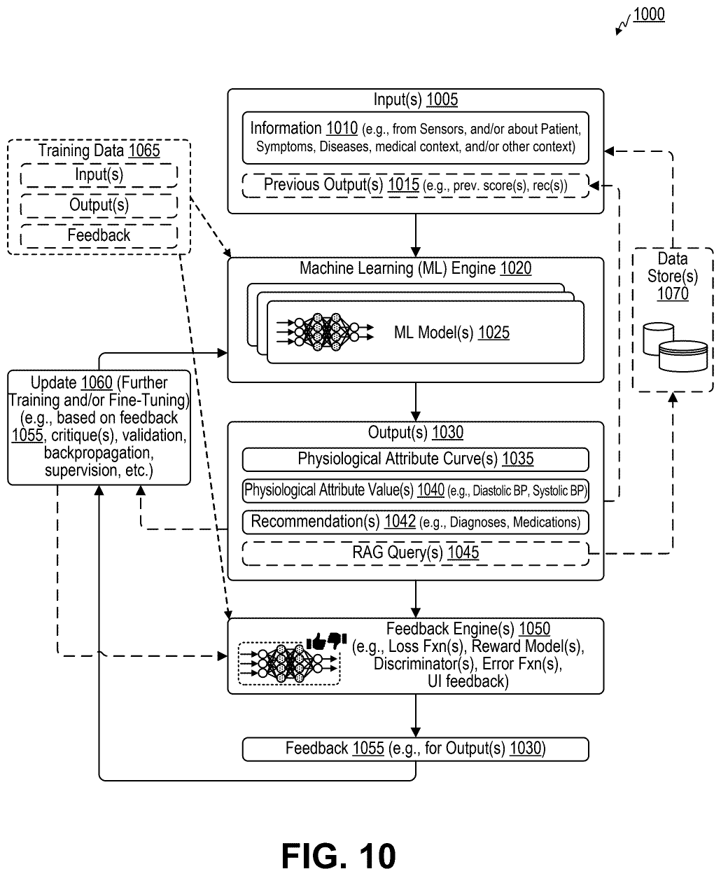

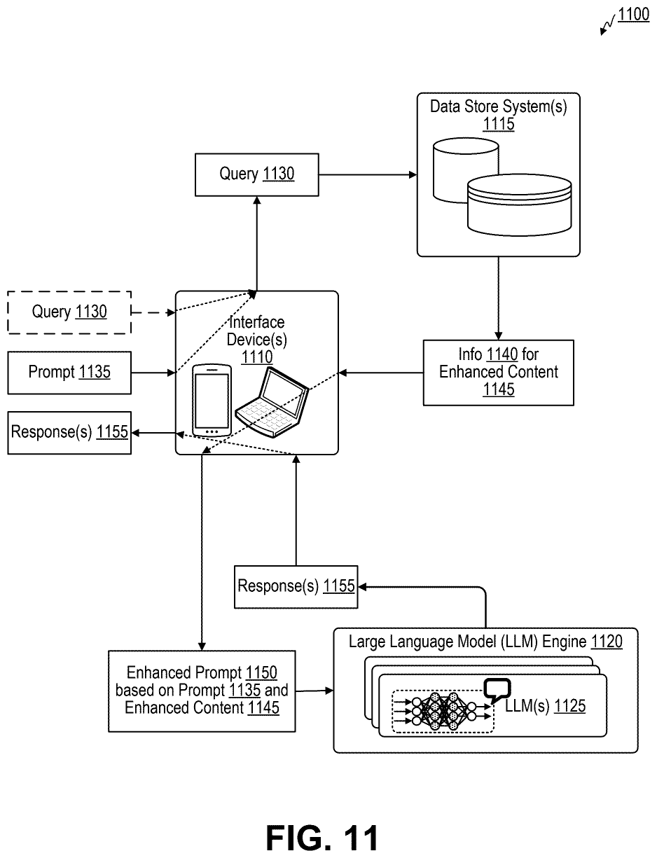

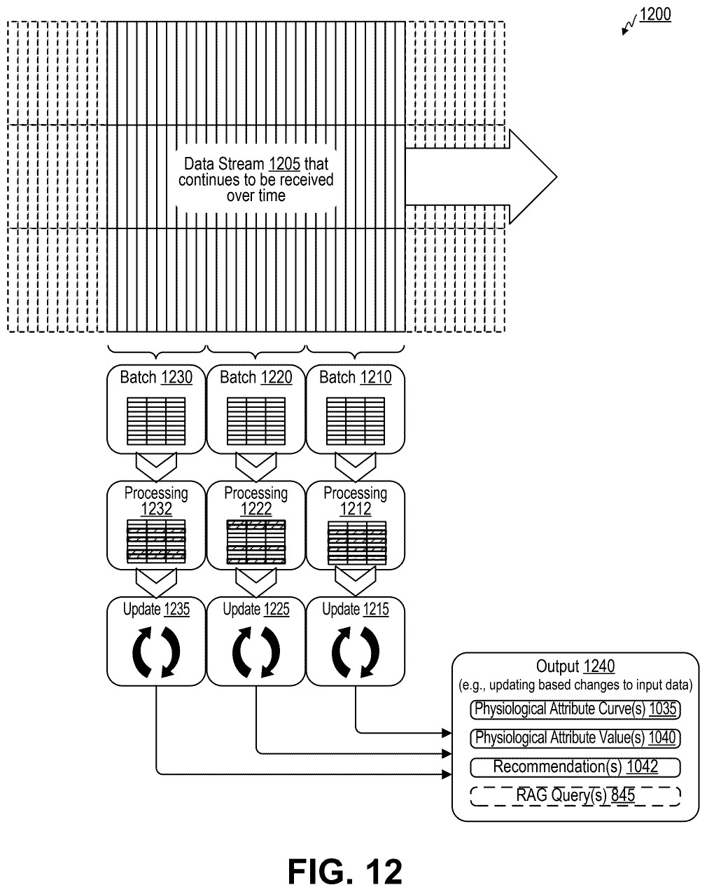

In some examples, the monitoring system 120 can use the artificial intelligence algorithm(s) 158 to determine, predict, and/or estimate certain aspects of the patient's blood pressure parameters (e.g., blood pressure curves, blood pressure values, and/or recommendations) or other bodily measurements and signals (i.e. heart rate) based on information about the patient (e.g., medical records, medical history, and/or other context), based on sensor data from the sensor(s) (e.g., the sensor(s) 134, the sensor(s) 148, and/or the sensor(s) 152), based on medical data (e.g., from medical textbooks, medical journals, and/or other repositories of medical information), symptoms entered through an interactive user interface of the monitoring system 120 (e.g., through the I/O interface(s) 160), mood of the patient 105, weather, atmospheric pressure, height of the physiological attribute monitoring system 100 (e.g., the monitoring system 120) above the sea level, atmospheric temperature, temperature of the gas in tubing or portion(s) (e.g., gas pillow(s) 126, gas bladder(s) 124) of the cuff 110 and/or other systems (e.g., central pressure system, gas canisters), pressure in compressed gas canister(s) 142, pressure in a central pressure system delivering gas to or through the pneumatic system 115 to the cuff 110, characteristics of the patient 105, characteristics of another patient with shared characteristic(s) with the patient 105, data associated with another patient using the same type of physiological attribute monitoring system 100, characteristics of and/or data from the physiological attribute monitoring system 100 and/or any other systems coupled to the physiological attribute monitoring system 100, characteristics of and/or data from the sensors(s) (e.g., the sensor(s) 134, the sensor(s) 148, and/or the sensor(s) 152), characteristics of and/or data from interactive user interface(s) of the physiological attribute monitoring system 100 (e.g., interactive user interface(s) that receive inputs and/or provide outputs through the I/O interface(s) 160), or combinations thereof. Examples of the artificial intelligence algorithm(s) 158 are illustrated in FIG. 10, FIG. 11, and FIG. 12.

In some examples, the system may include more than one cuff 110. Each cuff 110 can include its own set of fastener(s) 122, gas bladder(s) 124, gas pillow(s) 126, tube(s) 128, valve(s) 130, actuator(s) 132, and/or sensor(s) 134. In some examples, certain components can be shared between cuffs, such as certain sensor(s) 134 and/or certain tube(s) 128. Each cuff 110 can also have its own pneumatic system 115, monitoring system 120, and/or pneumatic system controller 150. In some examples, two or more cuffs can share a pneumatic system 115, monitoring system 120, and/or pneumatic system controller 150. In an illustrative example, a monitoring system 120 can monitor multiple cuffs 110, with each cuff 110 having its own pneumatic system 115 and its own pneumatic system controller 150 in the monitoring system 120. In another illustrative example, each cuff 110 of a set of cuffs can have its own pneumatic system 115 and its own monitoring system 120. In another illustrative example, a single pneumatic system 115 can supply gas to multiple cuffs 110, whether each cuff 110 has its own monitoring system 120 or some of the cuffs 110 share a monitoring system 120.

The monitoring system 120 also include input/output (I/O) interface(s) 160 such as displays (e.g., touchscreen displays with touch-sensitive surfaces), keyboards, keypads, mice, styluses, and the like. The I/O interface(s) 160 of the monitoring system 120 can be used to receive input(s) through interactive user interface(s) of the monitoring system 120 (e.g., providing contextual information about the patient 105 from the patient 105 and/or a physician or nurse), can be used to provide output(s) through interactive user interface(s) of the monitoring system 120 (e.g., providing blood pressure values for the patient 105, and/or recommendation(s) for the patient 105, to the patient 105 and/or a physician or nurse).

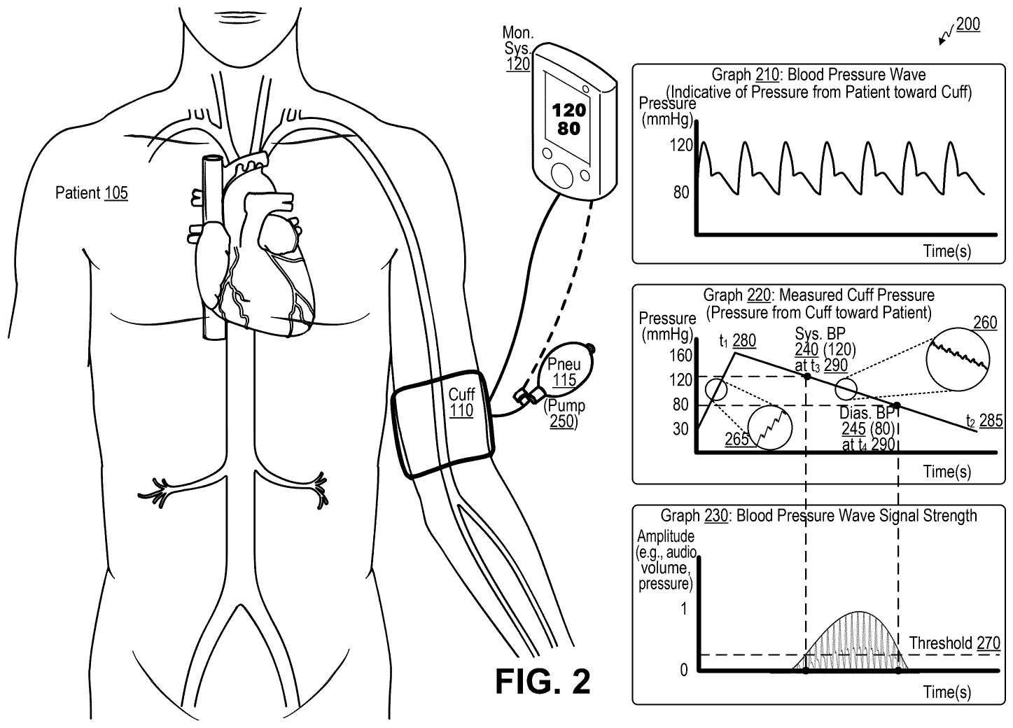

FIG. 2 is a block diagram illustrating use of a physiological attribute monitoring system 200 that determines systolic blood pressure 240 and diastolic blood pressure 245 during deflation of a cuff 110 that is previously inflated using a pump 250. The physiological attribute monitoring system 200 is an example of the physiological attribute monitoring system 100. The pump 250 is an example of the pump(s) 140.

A graph 210 represents a blood pressure pulse wave over time, which can be detected using sensor(s) of the physiological attribute monitoring system 200 (e.g., the sensor(s) 134, the sensor(s) 148, and/or the sensor(s) 152). The horizontal axis of the graph 210 represents time, moving forward from left to right. The vertical axis of the graph 210 represents pressure, measured in mmHg. The blood pressure pulse wave measured in the graph 210 is indicative of pressure from the patient 105 toward the cuff 110 (e.g., the blood pressure of the patient 105), with oscillations in blood pressure (e.g., oscillometric signal) between the systolic blood pressure 240 of the patient 105 (120 mmHg) and the diastolic blood pressure 245 of the patient 105 (80 mmHg). Alternately, the vertical axis of the graph 210 can represent audio volume or another amplitude measurement, with the blood pressure pulse wave representing Korotkoff audio recorded at or near the cuff 110 and/or the patient 105.

A graph 220 represents measured cuff pressure over time, which can be detected using sensor(s) of the physiological attribute monitoring system 200 (e.g., the sensor(s) 134, the sensor(s) 148, and/or the sensor(s) 152). The graph 220 illustrates that the pump 250 is used to inflate the cuff 110 from a deflated state from a time 0 to a time t₁ 280. The zoomed-in view 265 of the graph 220 of the pressure shows that this increase in pressure (from time 0 to time t₁ 280) is rough (e.g., non-smooth), with peaks and troughs that can be caused by discontinuities (and/or lack of continuity and/or consistency) in the flow of gas into the cuff 110 from the pump 250. Such discontinuities can be caused by motion of piston(s) and/or impeller(s) in the pump 250, time between pumping actions, or a combination thereof.

In some examples, the peaks and troughs can also be partly caused by oscillations in pressure from the blood pressure pulse wave (e.g., of the graph 210 and/or the graph 230). However, because of the discontinuities (and/or lack of continuity and/or consistency) in the flow of gas into the cuff 110 from the pump 250 can be inconsistent, it is difficult to identify which variations in the inflation pressure curve are caused by the discontinuities (and/or lack of continuity and/or consistency) in the flow of gas into the cuff 110 from the pump 250, and which variations in the inflation pressure curve are caused by the oscillations in pressure from the blood pressure pulse wave. In addition, in some examples, the pump 250 can introduce audio noise, making it more difficult to accurately monitor audio recordings for Korotkoff audio.

From time t₁ 280 to time t₂ 285, the pneumatic system 115 and/or the monitoring system 120 causes the cuff 110 to deflate, for instance by activating an actuator (e.g., actuator(s) 132 and/or actuator(s) 146) to open one or more valve(s) in the cuff 110 and/or in the pneumatic system 115 (e.g., valve(s) 130 and/or valve(s) 144). The deflation of the cuff 110 from time t₁ 280 to time t₂ 285 can be performed at a more controlled rate, steady rate, and/or constant rate, since deflation can be performed without a pump, through opening of one or more valve(s). Because of this, variations in pressure, and/or audio signals corresponding to Korotkoff audio, are easier to detect during the deflation of the cuff 110 (from time t₁ 280 to time t₂ 285) than during inflation of the cuff 110 (from time 0 to time t₁ 280).

The decrease in pressure in the cuff 110 is generally smooth, steady, constant, controlled, and/or consistent between t₁ 280 and time t₃ 290, and between t₄ 295 and time t₄ 285. However, as illustrated in the zoomed-in view 265 of the graph 220, between a time t₃ 290 to time t₄ 295, the decrease in pressure is rough (e.g., non-smooth), with peaks and troughs that can be caused by oscillations in pressure from the blood pressure pulse wave (e.g., of the graph 210 and/or the graph 230).

A graph 230 represents measures strength (e.g., amplitude and/or magnitude) of the blood pressure wave signal (e.g., oscillometric signal and/or Korotkoff audio signal) over time, for instance as detected using sensor(s) of the physiological attribute monitoring system 200 (e.g., the sensor(s) 134, the sensor(s) 148, and/or the sensor(s) 152). The graph 230, too, shows that the strength of the blood pressure pulse wave as detected by the sensor(s) exceeds a threshold 270 between the time t₃ 290 and the time t₄ 295. This is used by the physiological attribute monitoring system 200 to determine the systolic blood pressure 240 and the diastolic blood pressure 245. In particular, the pressure at the time t₃ 290 (where the pressure is higher than at the time t₄ 295) is identified as the systolic blood pressure 240 of the patient 105, while the pressure at the time t₄ 295 (where the pressure is lower than at the time t₃ 290) is identified as the diastolic blood pressure 245 of the patient 105. In the example of FIG. 2, the systolic blood pressure 240 of the patient 105 is illustrated to be 120 mmHg, while the diastolic blood pressure 245 of the patient 105 is illustrated to be 80 mmHg.

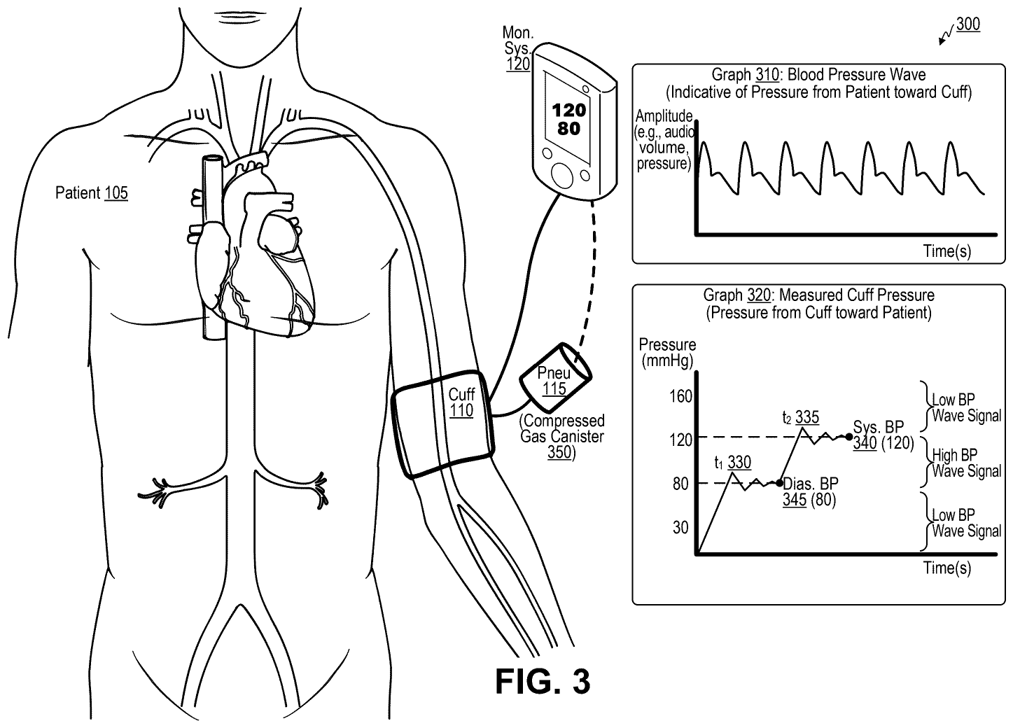

FIG. 3 is a block diagram illustrating use of a physiological attribute monitoring system 300 that determines systolic blood pressure 340 and/or diastolic blood pressure 345 based on convergence upon threshold(s) associated with these blood pressure values, and with a cuff 110 that is inflated using a compressed gas canister 350. The physiological attribute monitoring system 200 is an example of the physiological attribute monitoring system 100. The compressed gas canister 350 is an example of the compressed gas canister(s) 142.

Because the pneumatic system 115 of the physiological attribute monitoring system 300 supplies gas to the cuff 110 from the compressed gas canister 350 rather than the pump 250, the increase in pressure in the cuff 110 during inflation of the cuff 110 of the physiological attribute monitoring system 300 is more smooth, steady, constant, controlled, and/or consistent than the increase in pressure in the cuff 110 during inflation of the cuff 110 of the physiological attribute monitoring system 200. The physiological attribute monitoring system 300 can also supply gas to inflate the cuff 110 more quietly using the compressed gas canister 350 than the physiological attribute monitoring system 200 can using the pump 250. In some examples, the compressed gas canister 350 can be a portable compressed gas canister 350. In some examples, the compressed gas canister 350 can be part of a central gas system and/or central pressure system. Because of the lack of pump-related pressure discontinuities during inflation, and the lack of pump-related noise (that can prevent accurate monitoring of Korotkoff audio), the physiological attribute monitoring system 300 does not need to wait for deflation to determine the systolic blood pressure 340 or the diastolic blood pressure 345, but can check for the systolic blood pressure 340 and/or the diastolic blood pressure 345 during inflation, deflation, or both.

Similarly to the graph 210, the graph 310 represents a blood pressure pulse wave over time, which can be detected using sensor(s) of the physiological attribute monitoring system 200 (e.g., the sensor(s) 134, the sensor(s) 148, and/or the sensor(s) 152).

Similarly to the graph 220, the graph 320 represents measured cuff pressure over time, which can be detected using sensor(s) of the physiological attribute monitoring system 200 (e.g., the sensor(s) 134, the sensor(s) 148, and/or the sensor(s) 152). The graph 320 illustrates that the monitoring system 120 (e.g., the pneumatic system controller 150) and/or the pneumatic system 115 activates actuator(s) (e.g., actuator(s) 132, actuator(s) 146) to open valve(s) (e.g., valve(s) 130, valve(s) 144) to supply pressure from the compressed gas canister 350 to the cuff 110 to inflate the cuff 110 from a time 0 to a time t₁ 330.

The physiological attribute monitoring system 300 conveys gas from the compressed gas canister 350 to the cuff 110 to inflate the cuff 110 while a characteristic of the blood pressure wave signal (e.g., oscillometric signal and/or Korotkoff audio signal) is low (e.g., amplitude below an amplitude threshold) and/or is decreasing sufficiently (e.g., slope below a slope threshold), and until the characteristic of the blood pressure wave signal increases to be high (e.g., amplitude above the amplitude threshold) and/or is increasing sufficiently (e.g., slope above the slope threshold) at time t₁ 330. The amplitude threshold can be, for instance, the threshold 270 illustrated in the graph 230. The physiological attribute monitoring system 300 then knows that the diastolic blood pressure 345 is below the local peak pressure reached at time t₁ 330, which can be referred to as an upper bound pressure for the diastolic blood pressure 345. The physiological attribute monitoring system 300 then activates (e.g., actuator(s) 132, actuator(s) 146) to open valve(s) (e.g., valve(s) 130, valve(s) 144) to release gas from the cuff 110 to deflate the cuff 110 while the characteristic of the blood pressure wave signal is high (e.g., amplitude above the amplitude threshold) and/or is increasing sufficiently (e.g., slope above a slope threshold), and until the characteristic of the blood pressure wave signal decreases to be low (e.g., amplitude below the amplitude threshold) and/or is decreasing sufficiently (e.g., slope below a slope threshold). The physiological attribute monitoring system 300 then knows that the diastolic blood pressure 345 is above the local trough pressure reached at this time, which can be referred to as a lower bound pressure for the diastolic blood pressure 345.

The physiological attribute monitoring system 300 can then inflate the cuff 110 once more (e.g., at a slower rate and/or pressure than before for increased accuracy), while a characteristic of a blood pressure wave signal is low (e.g., amplitude below the amplitude threshold) and/or is decreasing sufficiently (e.g., slope below a slope threshold), and until the characteristic of the blood pressure wave signal increases to be high (e.g., amplitude above the amplitude threshold) and/or is increasing sufficiently (e.g., slope above a slope threshold), or until the pressure increases by half of (or another fraction or percentage of) difference (the “distance”) between the current lower bound and the current upper bound. The physiological attribute monitoring system 300 can thus reduce the upper bound for the diastolic blood pressure 345 to match the pressure of the cuff 110 at this time, which should be closer to the actual value for the diastolic blood pressure 345 due to the slower inflation rate. The physiological attribute monitoring system 300 can then deflate the cuff 110 once more (e.g., at a slower rate and/or pressure than before for increased accuracy), while a characteristic of a blood pressure wave signal is high (e.g., amplitude above the amplitude threshold) and/or is increasing sufficiently (e.g., slope above a slope threshold), and until the characteristic of the blood pressure wave signal decreases to be low (e.g., amplitude below the amplitude threshold) and/or is decreasing sufficiently (e.g., slope below a slope threshold), or until the pressure decreases by half of (or another fraction or percentage of) difference (the “distance”) between the current upper bound and the current lower bound. The physiological attribute monitoring system 300 can thus increase the lower bound for the diastolic blood pressure 345 to match the pressure of the cuff 110 at this time, which should be closer to the actual value for the diastolic blood pressure 345 due to the slower deflation rate. In some examples, the physiological attribute monitoring system 300 can perform a binary search for the diastolic blood pressure 345 by finding upper bounds and lower bounds for the diastolic blood pressure 345, and continuing to refine these upper bounds and lower bounds over time as the physiological attribute monitoring system 300 continues to gather more data through more pressure adjustments in the vicinity of the diastolic blood pressure 345, as discussed herein. In some examples, the binary search can stop early in case the system is able to measure the physiological attribute during the binary search.

This process can continue until the physiological attribute monitoring system 300 converges on a value for the diastolic blood pressure 345. For instance, once the difference between the upper bound and the lower bound (e.g., difference 410) is sufficiently small (e.g., below a difference threshold 420), the physiological attribute monitoring system 300 can identify the diastolic blood pressure 345 to be a middle value between the upper bound and the lower bound (e.g., the mean of the upper bound and the lower bound).

The physiological attribute monitoring system 300 can use a similar process to determine the systolic blood pressure 340. The physiological attribute monitoring system 300 conveys gas from the compressed gas canister 350 to the cuff 110 to inflate the cuff 110 while a blood pressure wave signal (e.g., oscillometric signal and/or Korotkoff audio signal) is high (e.g., amplitude above the amplitude threshold) and/or is increasing sufficiently (e.g., slope above a slope threshold), and until the characteristic of the blood pressure wave signal decreases to be low (e.g., amplitude below the amplitude threshold) and/or is decreasing sufficiently (e.g., slope below a slope threshold) at time t₂ 335. The physiological attribute monitoring system 300 then knows that the systolic blood pressure 340 is below the local peak pressure reached at time t₂ 335, which can be referred to as an upper bound pressure for the systolic blood pressure 340. The physiological attribute monitoring system 300 then activates (e.g., actuator(s) 132, actuator(s) 146) to open valve(s) (e.g., valve(s) 130, valve(s) 144) to release gas from the cuff 110 to deflate the cuff 110 while the characteristic of the blood pressure wave signal is low (e.g., amplitude below the amplitude threshold) and/or is decreasing sufficiently (e.g., slope below a slope threshold), and until the characteristic of the blood pressure wave signal increases to be high (e.g., amplitude above the amplitude threshold) and/or is increasing sufficiently (e.g., slope above a slope threshold). The physiological attribute monitoring system 300 then knows that the systolic blood pressure 340 is above the local trough pressure reached at this time, which can be referred to as a lower bound pressure for the systolic blood pressure 340.

The physiological attribute monitoring system 300 can then inflate the cuff 110 once more (e.g., at a slower rate and/or pressure than before for increased accuracy), while a characteristic of a blood pressure wave signal is high (e.g., amplitude above the amplitude threshold) and/or is increasing sufficiently (e.g., slope above a slope threshold), and until the characteristic of the blood pressure wave signal decreases to be low (e.g., amplitude below the threshold) and/or is decreasing sufficiently (e.g., slope below a slope threshold), or until the pressure increases by half of (or another fraction or percentage of) difference (the “distance”) between the current lower bound and the current upper bound. The physiological attribute monitoring system 300 can thus reduce the upper bound for the systolic blood pressure 340 to match the pressure of the cuff 110 at this time, which should be closer to the actual value for the diastolic blood pressure 345 due to the slower inflation rate. The physiological attribute monitoring system 300 can then deflate the cuff 110 once more (e.g., at a slower rate and/or pressure than before for increased accuracy), while a characteristic of a blood pressure wave signal is low (e.g., amplitude below the amplitude threshold) and/or is decreasing sufficiently (e.g., slope below a slope threshold), and until the characteristic of the blood pressure wave signal increases to be high (e.g., amplitude above the amplitude threshold) and/or is increasing sufficiently (e.g., slope above a slope threshold, or until the pressure decreases by half of (or another fraction or percentage of) difference (the “distance”) between the current upper bound and the current lower bound. The physiological attribute monitoring system 300 can thus increase the lower bound for the systolic blood pressure 340 to match the pressure of the cuff 110 at this time, which should be closer to the actual value for the diastolic blood pressure 345 due to the slower deflation rate. In some examples, the physiological attribute monitoring system 300 can perform a binary search for the systolic blood pressure 340 by finding upper bounds and lower bounds for the systolic blood pressure 340, and continuing to refine these upper bounds and lower bounds over time as the physiological attribute monitoring system 300 continues to gather more data through more pressure adjustments in the vicinity of the systolic blood pressure 340, as discussed herein.

This process can continue until the physiological attribute monitoring system 300 converges on a value for the systolic blood pressure 340. For instance, once the difference between the upper bound and the lower bound (e.g., difference 410) is sufficiently small (e.g., below a difference threshold such as the difference threshold 420), the physiological attribute monitoring system 300 can identify the systolic blood pressure 340 to be a middle value between the upper bound and the lower bound (e.g., the mean of the upper bound and the lower bound).

The process used by the physiological attribute monitoring system 300 to determine the systolic blood pressure 340 and the diastolic blood pressure 345 (e.g., binary search and/or another convergence-based search) is significantly faster than the process used by the physiological attribute monitoring system 200 to determine the systolic blood pressure 240 and the diastolic blood pressure 245. For instance, in some examples, the physiological attribute monitoring system 300 can determine the systolic blood pressure 340 and the diastolic blood pressure 345 in approximately one or more seconds. On the other hand, in some examples, the physiological attribute monitoring system 200 to determine the systolic blood pressure 240 and the diastolic blood pressure 245 in approximately 2 minutes. In some examples, the binary search can stop early in case the system is able to measure the physiological attribute during the binary search.

The process used by the physiological attribute monitoring system 300 to determine the systolic blood pressure 340 and the diastolic blood pressure 345 is also more accurate than the process used by the physiological attribute monitoring system 200 to determine the systolic blood pressure 240 and the diastolic blood pressure 245. For instance, the physiological attribute monitoring system 200 does not slow or reverse deflation of the cuff 110 from time t₁ 280 to time t₂ 285, and thus only gets one pass at collecting pressure readings (e.g., see graph 220) and/or blood pressure wave signal strength readings (e.g., see graph 230) to determine the systolic blood pressure 240 and the diastolic blood pressure 245. On the other hand, the physiological attribute monitoring system 300 collects multiple data points for pressures where the blood pressure wave signal is on either side of the amplitude threshold (e.g., threshold 270), and crosses over the amplitude threshold.

In some examples, instead of (or in addition to) using the compressed gas canister 350, the physiological attribute monitoring system 300 can receive pressurized gas from a central gas system and/or central pressure system. In some examples, instead of (or in addition to) using the compressed gas canister 350, the physiological attribute monitoring system 300 can use a pump system that provides a continuous or constant flow rate, such as circulation pump, a transfer pump, or a pump with multiple pistons that work in parallel to eliminate flow drop-offs or inconsistencies.

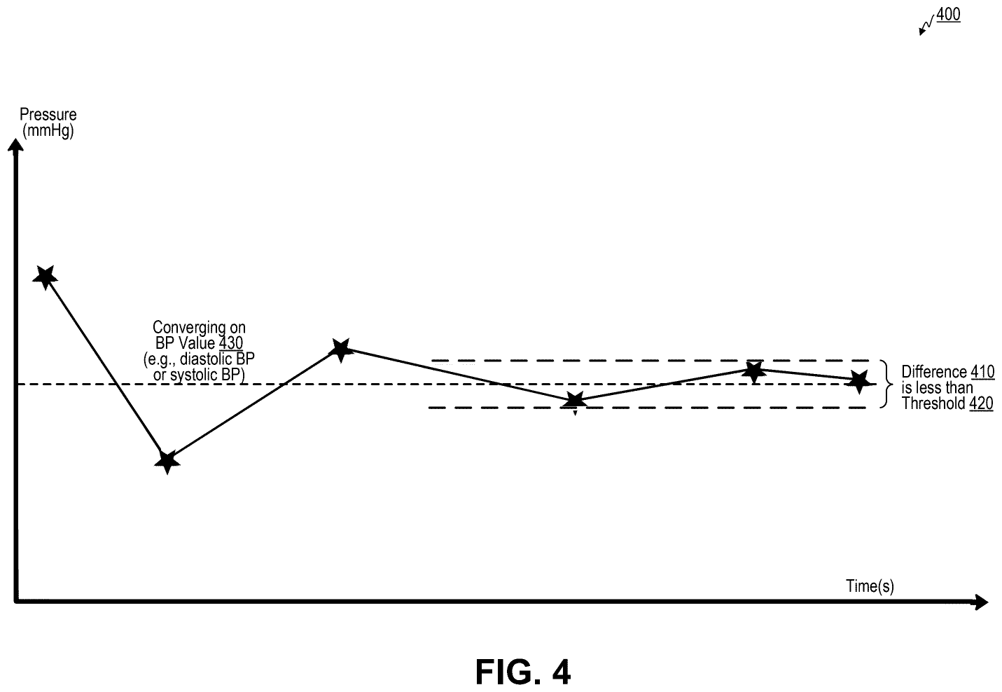

FIG. 4 is a graph diagram illustrating changes to cuff pressure in a process 400 involving sequential inflations and deflations of a cuff 110 of a physiological attribute monitoring system 300 to converge on a cuff pressure value indicative of a blood pressure value (e.g., systolic blood pressure 340 or diastolic blood pressure 345) of a patient 105. The graph of FIG. 4 illustrates the above-described process 400 for determining the systolic blood pressure 340 and the diastolic blood pressure 345, also illustrated in the graph 320.

When the physiological attribute monitoring system 300 is determining the diastolic blood pressure 345, the amplitude of the blood pressure wave signal (e.g., as in the graph 230) is low (below an amplitude threshold such as the threshold 270) while the pressure is below the diastolic blood pressure 345, and the amplitude of the blood pressure wave signal is high (above the amplitude threshold) while the pressure is above the diastolic blood pressure 345. This allows the physiological attribute monitoring system 300 to continue inflating and deflating the cuff 110 and noting at which pressures the amplitude of the blood pressure wave signal crosses the threshold to determine increasingly accurate and close upper bounds and lower bounds for the diastolic blood pressure 345, respectively, until the difference 410 between the upper bound and the lower bound is less than a difference threshold 420 for at least a predetermined amount of measurements (e.g., one or more measurements), and/or for at least a predetermined amount of time. In some examples, the difference threshold 420 can be predetermined or pre-set. In some examples, the difference threshold 420, and/or the time or number of measurements for which the difference 410 is to be below the difference threshold 420, can be dynamically determined based on noise level detected by the sensors (e.g., as a high noise level exceeding a noise threshold can make it more difficult to converge within a narrow difference threshold 420), attributes of the patient 105 (e.g., is the patient 105 moving, is the patient 105 sick, age of the patient 105, and/or other patient characteristics discussed herein), or a combination thereof.

When the physiological attribute monitoring system 300 is determining the systolic blood pressure 340, the amplitude of the blood pressure wave signal (e.g., as in the graph 230) is high (above the amplitude threshold) while the pressure is below the systolic blood pressure 340, and the amplitude of the blood pressure wave signal is low (below the amplitude threshold) while the pressure is above the systolic blood pressure 340. This allows the physiological attribute monitoring system 300 to continue inflating and deflating the cuff 110 and noting at which pressures the amplitude of the blood pressure wave signal crosses the threshold to determine increasingly accurate and close upper bounds and lower bounds for the systolic blood pressure 340, respectively, until the difference 410 between the upper bound and the lower bound is less than a difference threshold 420 for at least a predetermined amount of measurements (e.g., one or more measurements), and/or for at least a predetermined amount of time. In some examples, the difference threshold 420 can be predetermined or pre-set. In some examples, the difference threshold 420, and/or the time or number of measurements for which the difference 410 is to be below the difference threshold 420, can be dynamically determined based on noise level detected by the sensors (e.g., as a high noise level exceeding a noise threshold can make it more difficult to converge within a narrow difference threshold 420), attributes of the patient 105 (e.g., is the patient 105 moving, is the patient 105 sick, age of the patient 105, and/or other patient characteristics discussed herein), or a combination thereof . . .

FIG. 5 is a block diagram illustrating a physiological attribute monitoring system 500 with a cuff 110 around a body part 505 of a patient 105. The cuff 110 is divided (along a length of the body part 505) into multiple gas bladders 515A-515E that can be inflated and/or deflated independently. The gas bladders 515A-515E are further divided (along the circumference of the body part 505) into multiple gas pillows 530. The physiological attribute monitoring system 500 can be an example of the physiological attribute monitoring system 100. The gas bladders 515A-515E are examples of the gas bladder(s) 124 of the cuff 110. The gas pillows 530 are examples of the gas pillow(s) 126 of the cuff 110. The body part 505 may be, for example, an arm of the patient 105, a calf of the patient 105, a thigh of the patient 105, a leg of the patient 105, a wrist of the patient 105, a finger of the patient 105, another limb of the patient 105, another extremity of the patient 105, another body part of the patient 105, or a combination thereof. In an illustrative example, the body part 505 is an upper arm of the patient 105.

The cuff 110 is divided into gas bladders 515A-515E along a length of the body part 505 (e.g., along a length of the arm) of the patient 105, so that certain gas bladders are closer to the heart of the patient 105 and other gas bladders are farther from the heart of the patient 105. The cuff 110 is illustrated as including five gas bladders 515A-515E, but can have more or fewer than five in some examples. In some examples, the gas bladders 515A-515E are coupled to one another using a flexible and/or flaccid material. In some examples, the gas bladders 515A-515E are coupled to one another using tubing (e.g., tube(s) 128), valve(s) 130, and/or actuator(s) 132, so that gas can transfer from one gas bladder to another. In some examples, the gas bladders 515A-515E are not directly coupled to one another with any tubing (e.g., tube(s) 128,), valve(s) 130, and/or actuator(s) 132, so there is no gas transferred from one gas bladder to another.

The physiological attribute monitoring system 500 includes a pressure source 510, which may include, for example, the pump(s) 140 (e.g., the pump 250), the compressed gas canister(s) 142 (e.g., the compressed gas canister 350), another source of gas, another source of pressure, or a combination thereof. In some examples, the pressure source 510 is a source of positive pressure, for instance to provide pressure to inflate the gas bladders 515A-515E of the cuff 110. In some examples, the pressure source 510 is a source of negative pressure, for instance to provide suction to deflate the gas bladders 515A-515E of the cuff 110.

The physiological attribute monitoring system 500 includes valves 525A-525E. The pressure source 510 and/or the valves 525A-525E can be part of the pneumatic system 115 of the physiological attribute monitoring system 500. The valves 525A-525E can be examples of the valve(s) 130 and/or the valve(s) 144. In some examples, the physiological attribute monitoring system 500 includes actuator(s) (e.g., actuator(s) 132 and/or actuator(s) 146) that the physiological attribute monitoring system 500 can use to control (e.g., open or close) each of the valves 525A-525E. In some examples, the physiological attribute monitoring system 500 can control each of the valves 525A-525E independently of one another, for instance by controlling actuator(s) coupled to each of the valves 525A-525E independently from one another. In some examples, the monitoring system 120 (e.g., pneumatic system controller 150) and/or the pneumatic system 115 of the physiological attribute monitoring system 500 can open the valves 525A-525E to either inflate the gas bladders 515A-515E of the cuff 110 (e.g., to allow pressurized gas to flow from the pressure source 510 to the gas bladders 515A-515E of the cuff 110), or to deflate the gas bladders 515A-515E of the cuff 110 (e.g., to allow pressurized gas to flow out of the gas bladders 515A-515E of the cuff 110). In some examples, the monitoring system 120 (e.g., pneumatic system controller 150) and/or the pneumatic system 115 of the physiological attribute monitoring system 500 can close the valves 525A-525E to maintain the gas pressure within the gas bladders 515A-515E of the cuff 110.

In some examples, the physiological attribute monitoring system 500 includes sensors 520A-520E, which can include pressure sensors (e.g., pressure transducers), microphones or other audio sensors (e.g., audio transducers), optical sensors (e.g., optical transducers), bioimpedance sensors (e.g., bioimpedance transducers), mechanical sensors (e.g., mechanical transducers), other types of sensors discussed herein, or a combination thereof. The sensors 520A-520E can be examples of the sensor(s) 134, the sensor(s) 148, and/or the sensor(s) 152. The pressure sensors of the sensors 520A-520E can measure pressure at each of the gas bladders 515A-515E of the cuff 110 at a given time, and report the measured pressures to the monitoring system 120 for recording and analysis. The pressure sensors, audio sensors, optical sensors, bioimpedance sensors, and/or mechanical sensors of the sensors 520A-520E can measure readings for a blood pressure wave signal (e.g., oscillometric signal, Korotkoff audio signal, blood impedance change signals, blood volume change signals, and/or blood vibration signals), and can report these blood pressure wave signal readings to the monitoring system 120 for analysis.

In some examples, the pneumatic system 115 can inflate and/or deflate the different gas bladders 515A-515E uniformly. For instance, in some examples, the pneumatic system 115 can inflate the different gas bladders 515A-515E at uniform inflation rates and/or can deflate the different gas bladders 515A-515E at uniform deflation rates. In some examples, when inflating the cuff 110, the pneumatic system 115 can inflate the different gas bladders 515A-515E to the same pressure. In some examples, when deflating the cuff 110, the pneumatic system 115 can deflate the different gas bladders 515A-515E to the same pressure.

In some examples, the pneumatic system 115 can inflate and/or deflate the different gas bladders 515A-515E with variability in rates and/or pressures. For instance, in some examples, the pneumatic system 115 can inflate the different gas bladders 515A-515E at different inflation rates and/or can deflate the different gas bladders 515A-515E at different deflation rates. In some examples, when inflating the cuff 110, the pneumatic system 115 can inflate the different gas bladders 515A-515E to different pressures. In some examples, when deflating the cuff 110, the pneumatic system 115 can deflate the different gas bladders 515A-515E to different pressures.

For instance, in some examples, the physiological attribute monitoring system 500 inflates the gas bladder (of the gas bladders 515A-515E) that is most proximal to the heart to the lowest pressure (e.g., also inflating this gas bladder at a lower inflation rate or speed), and inflate the gas bladder (of the gas bladders 515A-515E) that is most distant from the heart to the highest pressure (e.g., also inflating this gas bladder at a higher inflation rate or speed). In some examples, this can allow the physiological attribute monitoring system 500 to test blood pressure wave signal readings for different pressures in parallel, further increasing speed of determining the blood pressure values (e.g., systolic blood pressure 340 and/or diastolic blood pressure 345) for the patient 105. For instance, in some examples, the physiological attribute monitoring system 500 can use lower-pressure gas bladders (that are closer to the heart) to determine the diastolic blood pressure 345 of the patient 105, while, in parallel, using the higher-pressure gas bladders (that are further from the heart) to determine the systolic blood pressure 340 of the patient 105. The higher-pressure gas bladder(s) being further from the heart can reduce the impact of the higher-pressure gas bladder(s) on blood pressure near the lower-pressure gas bladder(s), despite the higher-pressure gas bladder(s) having higher pressure. The lower-pressure gas bladders being at lower pressures than the higher-pressure gas bladder(s) can reduce the impact of the lower-pressure gas bladders on blood pressure near the higher-pressure gas bladder(s), despite the lower-pressure gas bladder(s) being closer to the heart of the patient 105.

In some examples, the order of the higher-pressure gas bladders and the lower-pressure gas bladders can be reversed, with the higher-pressure gas bladders being closer to the heart, and the lower-pressure gas bladders being farther from the heart. For instance, in some cases, this can help reduce pain for certain patients.

In an illustrative example of variable pressures in each of the gas bladders 515A-515E, the physiological attribute monitoring system 500 inflates the gas bladder 515A to about 60 mmHg, inflates the gas bladder 515B to about 90 mmHg, inflates the gas bladder 515C to about 120 mmHg, inflates the gas bladder 515D to about 150 mmHg, and inflates the gas bladder 515E to about 180 mmHg. The pressure differential between the gas bladders 515A-515E is maintained by the pressure valves 525A-525E. Once the gas bladders 515A-515E are inflated to these pressures, the pressure source 510 and/or the valves 525A-525E close delivery of the gas to the gas bladders 515A-515E. The monitoring system 120 then transduce the pressure in the gas bladders 515A-515E through the sensors 520A-520E (e.g., pressure transducers and/or pressure sensors) and read the blood pressure (e.g., take blood pressure wave signal readings). The monitoring system 120 can thus determine various waveforms affected by the patient's pulse, blood flow, movement, and/or artifacts.

For instance, if the systolic blood pressure of the patient 105 is 140 mmHg, the sensor 520E only registers minimal blood pressure wave signal, and/or non-variability of the pressure in time). In another example, if the diastolic blood pressure of the patient 105 is 90 mmHg, the sensor 520A can record minimal blood pressure wave signal, and/or non-variability of the pressure in time.

In some examples, each gas bladder of the gas bladders 515A-515E is further divided into gas pillows 530 along the circumference of the body part 505 (e.g., along the circumference of the arm) of the patient 105. For instance, as illustrated in FIG. 5, each gas bladder of the gas bladders 515A-515E is divided into seven gas pillows 530, but can have more or fewer than seven in some examples.

In some examples, the gas pillows 530 are coupled to one another using tubing (e.g., tube(s) 128), so that gas can transfer from one gas bladder to another. In some examples, the gas pillows 530 are not directly coupled to one another with any tubing (e.g., tube(s) 128), so there is no gas transferred from one gas bladder to another.

In some examples, the pneumatic system 115 can inflate and/or deflate the different gas pillows 530 uniformly. For instance, in some examples, the pneumatic system 115 can inflate the different gas pillows 530 at uniform inflation rates and/or can deflate the different gas pillows 530 at uniform deflation rates. In some examples, when inflating the cuff 110, the pneumatic system 115 can inflate the different gas pillows 530 to the same pressure. In some examples, when deflating the cuff 110, the pneumatic system 115 can deflate the different gas pillows 530 to the same pressure.

In some examples, the pneumatic system 115 can inflate and/or deflate the different gas pillows 530 with variability in rates and/or pressures. For instance, in some examples, the pneumatic system 115 can inflate the different gas pillows 530 at different inflation rates and/or can deflate the different gas pillows 530 at different deflation rates. In some examples, when inflating the cuff 110, the pneumatic system 115 can inflate the different gas pillows 530 to different pressures. In some examples, when deflating the cuff 110, the pneumatic system 115 can deflate the different gas pillows 530 to different pressures.

In some examples, the cuff 110 includes overlapping portions that overlap over each other while the cuff 110 is worn by the patient 105. For instance, if the cuff 110 includes fasteners that fasten one portion of the cuff 110 to another portion of the cuff 110, such as hook-and-loop fasteners (e.g., Velcro® fasteners), magnetic fasteners, or adhesive fasteners-a first portion of the cuff 110 (with a first set of fasteners) generally overlaps over a second portion of the cuff 110 (with a second set of fasteners that fasten to the first set of fasteners). In some examples, the fasteners include electromagnetic fasteners (e.g., with push button activation or controlled by the system). In some examples, the physiological attribute monitoring system 500 can inflate a first subset of the gas pillows 530 that are in one or both of these overlapping portions of the cuff 110 less than second subset of the gas pillows 530 that are not in any of the overlapping portions of the cuff 110. This can prevent the overlapping portion(s) of the cuff 110 from having increased gas volume (e.g., double gas volume, potentially impacting pressure) compared to the non-overlapping portions of the cuff 110. Only filling one of the overlapping portions can make the cuff 110 more reliable, accurate, and flexible-for instance by allowing the cuff 110 to more accurately accommodate and account for different patients having different sizes of the body part 505 This can prevent “double counting” pressure at overlapping portions of the cuff 110.

In some embodiments the cuff 110 includes a measure, or measuring tape, allowing operator to measure the body part 505 (e.g., upper arm) circumference. This information can be entered into the monitoring system 120 for its operations, AI algorithm(s) 158 and/or cloud systems, to be used in calibration, training, validation, and/or testing of the AI algorithm(s) 158—and/or in production to improve accuracy of estimation of physiological measures and signals such as blood pressure, or other physiological attributes. The gas bladder(s) 124, each separately or the entire cuff 110, can have automatic ways of determining of the how much of cuff 110 is overlapping (doubled), or what the body part 505 (e.g., arm) circumference is. For instance, the physiological attribute monitoring system 500 can use the magnetic or electromagnetic fasteners, and electromagnetic field sensors to determine the size of the body part 505. For instance, the position(s) of the fasteners relative to one another may be determined based on sensor data from magnetic fields detectors and/or electromagnetic fields detectors. The physiological attribute monitoring system 500 can determine and/or infer the circumferences of the body part 505 (e.g., arm) using pretrained AI/ML models on data. In some examples, the data that is used to pretrain these models, is obtained from previous operation of the physiological attribute monitoring system 500, but when body part (e.g., arm) circumference is known, or the same measurements are applied on body parts 505 (e.g., legs) with known circumference. The circumference of the body part 505 (e.g., arm or leg) can also be estimated, or measured, using math, statistics or AI/ML models. The AI/ML models for that would be pretrained on the data including, circumference of body parts (e.g., arms, legs), cuff pressure/volume curves, gas type used to fill the bladders, temperature of the gas within the bladders, inflation and deflation rate of the bladder, slope of pressure/volume curve during inflation and slope of pressure/volume curve during deflation volume, or other data discussed herein. The pretraining of these AI/ML models involves the use of data collected on various body parts (e.g., various patients arms, legs, and/or other body parts) with various circumferences, patients using various inflation/deflation rates and/or pressures, and various size cuffs, fastened at various strengths (e.g., tightness), with various combinations of bladders and or gas pillows, or combinations thereof. The AI/ML models, once trained, can be used to estimate the circumference of the body part 505 (e.g., arm). The same slope can used to determine and estimate other measurable bodily parameters or characteristics The data from cameras and digital video and audio recordings can be used to train system's AI not only for arm circumference, but also, for instance, to detect patient's movement, or restlessness. In some examples, the sensor(s) (e.g., the sensor(s) 134, the sensor(s) 148, and/or the sensor(s) 152), can include one or more cameras. In some examples, the camera(s) can be wide angle camera(s) coupled to the monitoring system 120, or in the room with the patient 105. In some examples, the camera(s) (and/or other sensor(s) 152) can be coupled to the monitoring system 120 through wired and/or wireless connection(s)). In some examples, the camera(s) can include visible light cameras, infrared (e.g., thermovision) cameras, night vision cameras, other trypes of cameras, or combinations thereof.

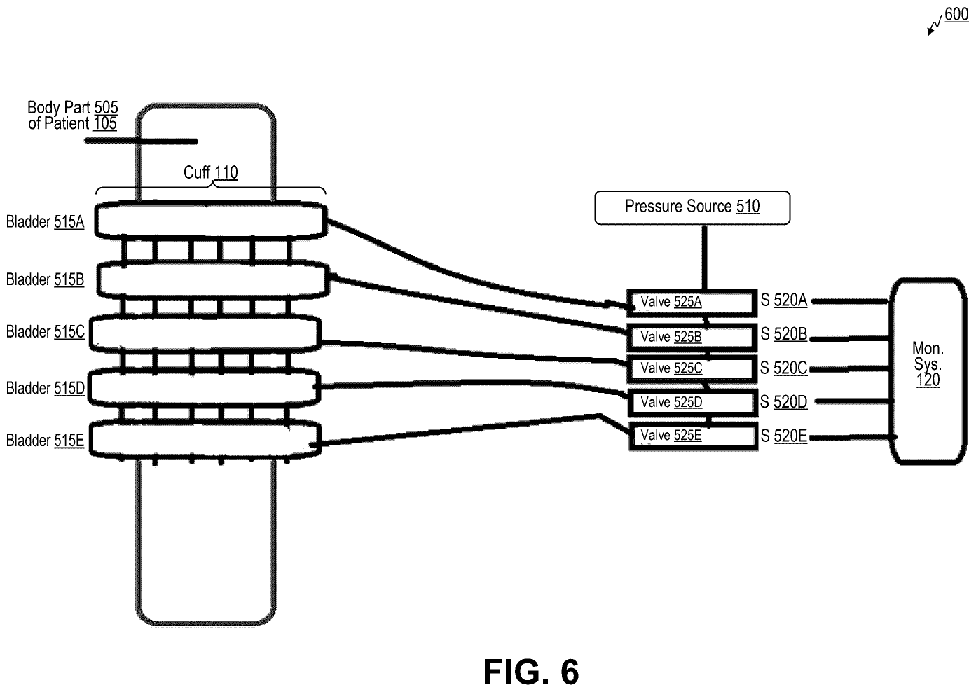

FIG. 6 is a block diagram illustrating a physiological attribute monitoring system 600 with a cuff 110 around a body part 505 of a patient 105. The cuff 110 is divided (along a length of the body part 505) into multiple gas bladders 515A-515E that can be inflated and/or deflated independently. The physiological attribute monitoring system 600 can be an example of the physiological attribute monitoring system 100. The physiological attribute monitoring system 600 is an example of a variant of the physiological attribute monitoring system 500 in which the gas bladders 515A-515E are not further divided into gas pillows 530.

Various signal characteristics can be measured by the sensors 520A-520E, including but not limited to: amplitude, minimal pressure, maximal pressure, length of peak, decay of peak, attack for peak and other their waveform parameters, principal component analysis, Fourier and Harmonic analysis of the waveform (e.g., during the normal or abnormal heart function), or a combination thereof. Heart pumping of the blood can be registered and quantified and digitalized at the monitoring system 120. The monitoring system 120 can also analyze blood pressure pulse waveforms by analyzing, for instance, amplitude statistics, variance measures, shape descriptors, energy and power calculations, Fourier transform methods, spectral moments, power spectrum metrics, spectral flatness and entropy, wavelet transforms, other transforms like Wigner Village distribution, statistical signal processing like correlation analysis, coherence analysis, envelope analysis, detrended fluctuation analysis, requirements quantification analysis, pattern recognition methods like principal component analysis or linear discriminant analysis, clustering algorithms, hidden Markov models, dynamic time warping, nonlinear dynamics metrics like fractal dimension or entropy measures, Lyapunov exponents, faces space reconstruction, statistical modeling like regression models, timeseries models for example autoregressive integrative moving average, state space models, Bayesian waveform analysis, deep learning, neural network, gradient boosting models, random forest, decision trees, machine learning (ML) algorithms, artificial intelligence (AI) algorithms, or any other formula, statistical or analytics system, or a combination thereof.

Before the blood pressure monitoring system (e.g., physiological attribute monitoring system 100, physiological attribute monitoring system 300, physiological attribute monitoring system 500, or physiological attribute monitoring system 600) is used to detect blood pressure values (e.g., systolic blood pressure 340, diastolic blood pressure 345) for a patient 105, the blood pressure monitoring system is trained, calibrated, and/or validated against ground truth or real known measured physiologic parameters or signals (e.g., blood pressure and/or heart rate). Based on this training, calibration, and/or validation, the blood pressure monitoring system is able to correlate signals and/or waveforms recorded of all or some sensors 520A-520E, including and not limited to, data stored in a cloud storage repository, past recordings from other databases, simulations, experiments with phantom patients (e.g., devices that simulate blood flowing through an arm), experiments with real patients (e.g., patient 105), or blood pressure/circulation simulators.

In some examples, before the blood pressure monitoring system (e.g., physiological attribute monitoring system 100, physiological attribute monitoring system 300, physiological attribute monitoring system 500, or physiological attribute monitoring system 600) is used to detect blood pressure values (e.g., systolic blood pressure 340, diastolic blood pressure 345) for a patient 105, one or more AI algorithm(s) 158 (e.g., ML models) are trained, validated, fine-tuned, calibrated, and/or updated using databases (or other data stores) which have ground truth readings of physiologic signals (e.g., blood pressure recorded by arterial line, or manual blood pressure checks by physicians, blood pressure recorded by left heart or aortic catheterization) and data from all or some sensors (e.g., sensor(s) 134, sensor(s) 148, sensor(s) 152, sensors 520A-520E) within the blood pressure monitoring system. By synchronizing and/or calibrating the sensor readings to the ground truth blood pressure recordings, the blood pressure monitoring system can improve accuracy of estimation of physiologic signals (e.g., blood pressure) based on data from all or some sensors within the blood pressure monitoring system.

Training, validation, fine-tuning, calibration, and/or updating of the blood pressure monitoring system (e.g., physiological attribute monitoring system 100, physiological attribute monitoring system 300, physiological attribute monitoring system 500, or physiological attribute monitoring system 600) (e.g., of the AI algorithm(s) 158) can be based on experiments and/or validation using various patients with various sizes of the body part 505 (e.g., arm), various patients with different blood pressures, various patients with different diseases and/or symptoms, various patients with different genders and/or sexes, various patients with different ethnicities and/or racial backgrounds, different body parts (e.g., arm vs. calf), or combinations thereof. This can help the blood pressure monitoring system be flexible and reliable in a large number of scenarios. For instance, if a patient 105 has injured arm(s) or does not have arm(s) (e.g., due to amputation or other injury), the blood pressure monitoring system can be used on the patient's calf, thigh, or another body part.

In some examples, the monitoring system 120 can analyze data from the sensors (e.g., sensor(s) 134, sensor(s) 148, sensor(s) 152, sensors 520A-520E) to identify patterns, identify trends, identify waveform characteristics, identify and remove outliers, generate statistical models, make predictions, make recommendations (e.g., of diagnoses and/or treatments), or a combination thereof. In some examples, the monitoring system 120 can route the sensor data to a server system or other remote system that can be used to perform some or all of this analysis, allowing the monitoring system 120, reducing computational load and power draw at the monitoring system 120 by offloading analyses that use more computational resources to the remote system. In some examples, the monitoring system 120 can maintain and/or transfer the waveforms and all recorded data from the sensors (e.g., sensor(s) 134, sensor(s) 148, sensor(s) 152, sensors 520A-520E) to a central repository of data, for instance where other medical records and/or data associated with the patient 105 may be stored. This central repository, in some examples, may be the remote system that performs the analyses. In some examples, offloading data and/or processing may also allow a combination of review using the AI algorithm(s) 158 and/or review using experts (e.g., physicians and/or nurses) to provide improved accuracy of diagnoses and/or treatment selections for the patient 105.

One technical improvement of the blood pressure monitoring system (e.g., physiological attribute monitoring system 100, physiological attribute monitoring system 300, physiological attribute monitoring system 500, or physiological attribute monitoring system 600) is continuous blood pressure monitoring without the need of placement of an invasive arterial line, or regular slow (minutes-long) inflation and deflation of the cuff 110. In some examples, the blood pressure monitoring system can be configured to continuously monitor one or both of the blood pressure values (e.g., diastolic blood pressure and/or systolic blood pressure). For instance, for certain surgeries and/or treatments (e.g., dialysis), monitoring of diastolic blood pressure is sufficient. The blood pressure monitoring system can continuously monitor blood pressure monitoring system by continuously converging on the diastolic blood pressure over time (e.g., as illustrated in the graph 320 and/or the graph of the process 400), continuing to inflate and deflate the cuff with periodic micro-adjustments. This can prevent having to increase pressure to potentially harmful levels that might cut off or slow blood flow (e.g., as in during measurement of the systolic blood pressure), thereby improving patient outcomes and many diseases and/or treatments, for example during cardiac surgery, dialysis, or in an intensive surgery (e.g., in the ICU). The blood pressure monitoring system continuing to monitor blood pressure in this way can also continue to improve accuracy of the blood pressure measurements over time, as the convergence can continue to make smaller and smaller adjustments to the pressure in the cuff 110 as illustrated in the graph 320 and the graph of the process 400 in FIG. 4.

The bound pressure searching (e.g., binary searching) in the process 400 of FIG. 4 is optimized with repeated measurements, as pressure rarely changes dramatically within the short span of time that such repeated measurements are taken in. If needed, and adjustment of bounds is needed (e.g., if blood pressure measuring in prior bounds fails due to change in blood pressure) the bounds can be adjusted, for full new establishment of new bounds. For instance, in some examples measurement for systolic blood pressure and/or diastolic blood pressure can be retriggered either when needed (e.g., when the blood pressure measurement is failing), when a user (e.g., the patient 105 or a physician or other operator) requests it, or periodically according to a predetermined interval (e.g., every 30 seconds, ever N seconds, every minute, every five minutes, every N minutes, ever hour, every N hours, or a combination thereof).

On the other hand, if subsequent measurements of a blood pressure value (e.g., diastolic blood pressure) succeed, the upper bound may be gradually decreased, and lower bound increased, so the meter will attempt to check blood pressure and/or other measurable bodily characteristics and measures with less time and less inflation/deflations. This adaptive bound mode of operation can be a default setting, or can be turned on or off as a separate mode as needed. For instance, this type of mode can work well if the patient 105 is sleeping, under anesthesia, undergoing dialysis, and/or if the 50//is only tracking diastolic blood pressure without tracking systolic blood pressure. This mode would be less likely to be useful for an agitated ICU patient in restraints, as such a patient may have rapidly fluctuating blood pressure values. This mode can be tuned, autotuned, or set by an operator (e.g., tuning or setting how aggressively the system decreases upper bounds and increases lower bounds to narrow bounds, and/or pushes bounds outward to extend bounds outward in case of a blood pressure check failure) based on a patient's daily schedule, daytime activity, night time activity, and sensor-measured activity levels (e.g., based for example on accelerometers reads). The mode can be turned on and off based on activity and/or other parameters, such as any of the parameters discussed herein.

In some examples, where the blood pressure monitoring system (e.g., physiological attribute monitoring system 100, physiological attribute monitoring system 300, physiological attribute monitoring system 500, or physiological attribute monitoring system 600) is configured to monitor only one blood pressure value (e.g., diastolic pressure or systolic blood pressure but not both) over time, this can also allow the physiological attribute monitoring system 500 to analyze sensor data from the sensors (e.g., sensor(s) 134, sensor(s) 148, sensor(s) 152, sensors 520A-520E) to monitor perfusion and/or blood pressures in different body parts, for instance even to monitor blood pressure in a body part that is currently being operated on (e.g., through surgery or another procedure) without cutting off or slowing blood flow. This can also improve patient outcomes during certain surgeries, such as treatment of wounds where the patient is at risk of significant blood loss during the surgery.

In some examples, where the blood pressure monitoring system (e.g., physiological attribute monitoring system 100, physiological attribute monitoring system 300, physiological attribute monitoring system 500, or physiological attribute monitoring system 600) is configured to monitor only one blood pressure value (e.g., diastolic pressure) over time, this can also allow the blood pressure monitoring system to monitor a body part 505 of the patient 105 while the body part 505 is undergoing dialysis with the fistula or arteriovenous (AV) graft on that body part 505. The blood pressure monitoring system can use the same body part 505 to monitor blood pressure (e.g., diastolic blood pressure) while hemodialysis is ongoing, using an AV fistula or AV graft on that body part 505. Blood that is at higher pressures than the pressure needed by the blood pressure monitoring system to estimate diastolic pressure (e.g., the vast majority of the patient's blood) will still get through the measurement area, distally into extremities (e.g., to AV fistula or AV graft), meaning that dialysis can continue even during continuous blood pressure monitoring using the blood pressure monitoring system. In some examples, the blood pressure monitoring system can monitor the blood pressure value (e.g., diastolic blood pressure) on multiple body parts, such as a body part that is undergoing dialysis (while the body part is undergoing dialysis) as well as a body part that is not undergoing dialysis, with the blood pressure monitoring system comparing and/or calibrating the blood pressure measurements from the different body parts to negate or minimize any potential impact of the dialysis itself on the patient's blood pressure value measurements. In some examples, blood pressure monitoring system can monitor the blood pressure value (e.g., diastolic blood pressure) on a body part having the AV fistula or AV graft in patients not undergoing hemodialysis at the time of blood pressure monitoring. The blood at higher pressures than pressure needed to estimate diastolic pressure would still get through the measurement area, distally into extremities.

As noted previously, in some examples, the gas bladders 515A-515E are further divided into different gas pillows 530, for instance as illustrated in FIG. 5. In some examples, each gas bladder can be divided into the same number of different gas pillows 530 as other pillows—for instance, in the physiological attribute monitoring system 500, all of the gas bladders 515A-515E are divided into seven gas pillows 530. In some examples, different gas bladders of the gas bladders 515A-515E can be divided into different numbers of gas pillows 530. For instance, the gas bladder 515A can be divided into seven gas pillows, the gas bladder 515B can be divided into ten gas pillows, and so forth. In some examples, the functional divisions between the different gas pillows 530 of each gas bladder are fully permissive, or partially permissive, of flow of the gas.

In some examples, the different gas bladders 515A-515E may have different lengths, widths, and/or sizes. For instance, different gas bladders 515A-515E can be designed at different sizes to accommodate various circumferences of the body part 505, for various sizes of patients. A blood pressure monitoring system with different lengths of the gas bladders 515A-515E can rely on a shorter (smaller) gas bladder for smaller patients (e.g., where the circumference of the body part 505 is smaller than a threshold) and can rely on a longer (larger) gas bladder for larger patients (e.g., where the circumference of the body part 505 is larger than the threshold).

In some examples, the gas bladders 515A-515E do not communicate between themselves. Their walls can be coupled to (e.g., connected with) the adjacent gas bladders with a soft, flexible, and/or flaccid material. This allows for maintenance of different gas pressures in different gas bladders 515A-515E, and also allows for each bladder and respective valve(s) and/or sensor(s) (e.g., pressure transducers and/or microphones) to work independently of each other, and independently react to the measured physiological values like systolic blood pressure, temperature, respiratory rate, heart rate, diastolic blood pressure, other types of sensor data and/or readings discussed herein, or a combination thereof.

In the examples of the physiological attribute monitoring system 500 and/or the physiological attribute monitoring system 600 illustrated in FIG. 5 and FIG. 6, respectively, the length of tubing between the valves 525A-525E and the gas bladders 515A-515E is relatively long, for illustrative purposes. However, in some examples, this tubing may be shorter or longer. In some examples, the tubing between the valves 525A-525E and the gas bladders 515A-515E can be included in, and/or encompassed in, one magistral, or multitube, or multi lumen tube or connector. This can avoid additional tangling of the tubes, and this minimize effects of the outside pressures on the tubing and the gas bladders 515A-515E.

Additionally, while the sensors 520A-520E of the physiological attribute monitoring system 500 and physiological attribute monitoring system 600 are illustrated as separated from the cuff 110 in FIG. 5 and FIG. 6, in some examples, at least some of the sensors 520A-520E can be positioned differently. For instance, in some examples, the sensors 520A-520E can be part of the cuff 110, for instance being coupled to (e.g., adherent to) the gas bladders 515A-515E. For instance, in some examples, the sensor 520A can be coupled to the gas bladder 515A, the sensor 520B can be coupled to the gas bladder 515B, and so forth. Similarly, in some examples, the valve 525A can be coupled to the gas bladder 515A, the valve 525B can be coupled to the gas bladder 515B, and so forth. This can reduce and/or shorten the amount of tubing, and decrease artifacts, including from movement of tubing, of the patient, and/or other movements—and/or reduce artifacts from external pressures, forces, and/or factors. In some examples, the monitoring system 120 can be coupled to all of the sensors 520A-520E, valves 525A-525E, and/or gas bladders 515A-515E. In some examples, the monitoring system 120 can be receive sensor data from the sensors 520A-520E, and can control (e.g., via the pneumatic system controller 150) when to open or close each of the valves 525A-525E, thereby controlling how much gas is in each of the gas bladders 515A-515E at a given time.

In some examples, the sensors (e.g., sensor(s) 134, sensor(s) 148, sensor(s) 152, sensors 520A-520E) of the blood pressure monitoring system (e.g., physiological attribute monitoring system 100, physiological attribute monitoring system 300, physiological attribute monitoring system 500, or physiological attribute monitoring system 600) can include, for instance, pressure sensors, temperature transducers/sensors, light sensors, electromagnetic sensors, sound sensors, spectroscopy sensors, pulse-oximetry sensors, oximetry sensors, cameras, microphones, current emitters, charge emitters, piezoelectric, microphones, cameras, light emitting diodes (LEDs), warming elements, cooling elements, conductive parts adherent to skin (of the patient 105), Doppler sensors, sound sensors, electromagnetic coils, electromagnetic sensors, light sensors, ultrasound sensors, ultrasound emitters, light emitters, electromagnetic field and wave emitters, other types of sensors discussed herein, other types of emitters discussed herein, or combinations thereof.

In some examples, the sensors, emitters, and/or conductive parts (adherent to skin) can be used by the blood pressure monitoring system to, for instance, record temperature waveform, pulse waveform, independence, inductance, capacitance, bioreactance, impedance, other characteristics or metrics associated with the patient 105, or a combination thereof. In some examples, the sensors, emitters, and/or conductive parts (adherent to skin) can be used by the blood pressure monitoring system to (1) generate signals or impulses or energy (e.g. electromagnetic wave, light, sound, current, electric charge, electromagnetic wave, sound or other); (2) sense the effects on generated signals or impulses or energy by physiological sources like systolic blood pressure, blood flow, pulse, temperature, respiratory rate, heart rate, diastolic pressure, body movement, artifacts, and/or background noise, and/or (3) sense the effects of physiological signals (e.g., heart beats, temperature, muscle generated electric potential) on generated signals (e.g., electric current, sound).

In some examples, the sensors (e.g., sensor(s) 134, sensor(s) 148, sensor(s) 152, sensors 520A-520E) of the blood pressure monitoring system include a temperature sensor and/or impedance sensor that the blood pressure monitoring system can use to detect patient movement. In some examples, the sensors (e.g., sensor(s) 134, sensor(s) 148, sensor(s) 152, sensors 520A-520E) of the blood pressure monitoring system include an electromagnetic sensor or impedance sensor that the blood pressure monitoring system can use to detect high amounts of background electromagnetic noise. In some embodiments, the detection of patient movement or high amount of background electric noise by the blood pressure monitoring system can could trigger the monitoring system 120 to output (e.g., through the I/O interface(s) 160) an alert (e.g., to an operator of the blood pressure monitoring system) alerting the operator of potential errors during blood pressure measurement. In some examples, the blood pressure monitoring system can automatically perform additional cycles of inflation and deflation in such circumstances to try to improve accuracy further (e.g., to try to converge more closely to the accurate systolic blood pressure or diastolic blood pressure) despite this. For instance, in some examples, the blood pressure monitoring system can automatically reduce the difference threshold 420 to try to improve accuracy in such situations. In some examples, the blood pressure monitoring system can instead increase the difference threshold 420, for instance if the added noise (e.g., from movement and/or electromagnetic noise) is making it impossible to converge on a blood pressure value (e.g., systolic blood pressure or diastolic blood pressure) at the default value for the difference threshold 420.

In some examples, the physiological attribute monitoring system (e.g., physiological attribute monitoring system 100, physiological attribute monitoring system 300, physiological attribute monitoring system 500, and/or physiological attribute monitoring system 600) is configured to repeat estimations and/or measurements of blood pressures and bodily characteristics and signal(s) multiple times (e.g., a prespecified or predetermined number of times, or an automatically determined number of times based on sensor data). For instance, the physiological attribute monitoring system can repeat measurements of physiological attributes (e.g., blood pressure values) periodically (e.g., at preset intervals or times, and/or automatically determined intervals or times), at specific times (e.g., scheduled times), until the asymptotic convergence happens of measured blood pressure or other bodily characteristics or until specific preset time lapses, until another event that can be automatically detected (e.g., patient 105 stands up from a sitting or laying down position, until patients moves or goes to sleep as determined by accelerometers) occurs, until a predetermined amount of time lapses (e.g., five minutes) without activity by the patient 105 (e.g., to ensure that the patient 105 is sufficiently rested for an accurate blood pressure reading), or a combination thereof.

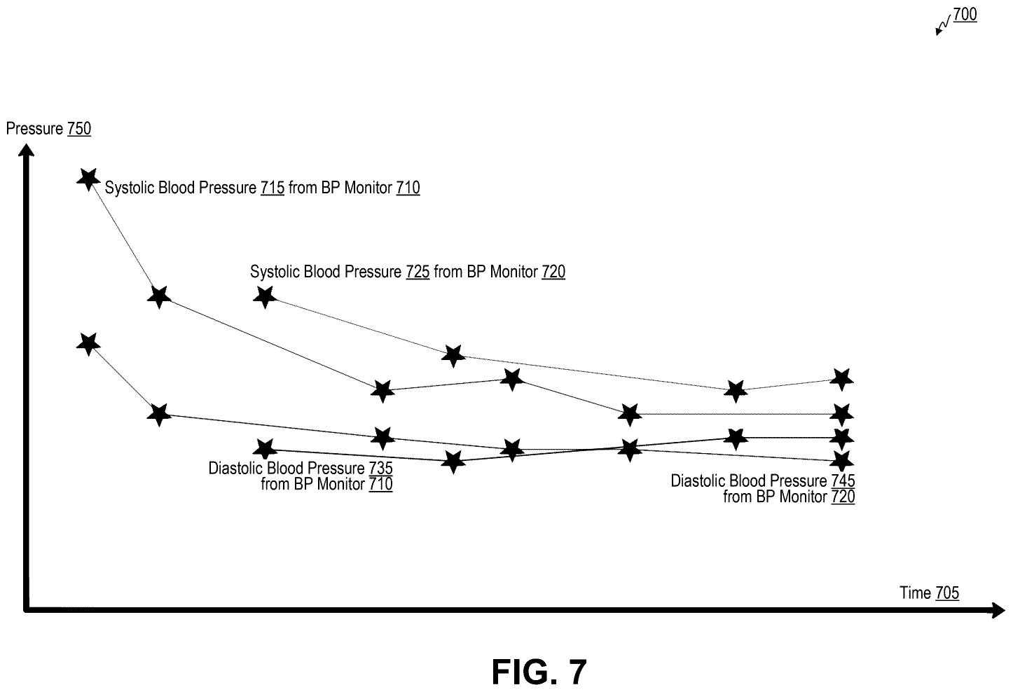

FIG. 7 is a graph diagram 700 illustrating a comparison between blood pressure values measured using two different blood pressure monitor systems, or two different sensors (associated with different cuffs or gas bladders) of a single blood pressure monitor system. In the graph diagram 700, the horizontal axis is time 705 (going forward from left to right), and the vertical axis is pressure 750, increasing from bottom to top. The graph diagram 700 illustrates values for systolic blood pressure and diastolic blood pressure from a first blood pressure monitor 710 and a second blood pressure monitor 720. In particular, the graph diagram 700 illustrates changes to systolic blood pressure 715 as measured by the first blood pressure monitor 710 over time, changes to systolic blood pressure 725 as measured by the second blood pressure monitor 720 over time, changes to diastolic blood pressure 735 as measured by the first blood pressure monitor 710 over time, and changes to diastolic blood pressure 745 as measured by the second blood pressure monitor 720 over time.

In some examples the first blood pressure monitor 710 and the second blood pressure monitor 720 are entirely separate and/or different blood pressure monitoring systems. In some examples, the first blood pressure monitor 710 and the second blood pressure monitor 720 share certain components, such as the monitoring system 120, the pneumatic system 115, and/or the cuff 110. For instance, in an illustrative example, the first blood pressure monitor 710 can correspond to a first gas bladder (e.g., gas bladder 515A), a first set of sensor(s) (e.g., sensor 520A), and/or a first valve (e.g., valve 525A), while the second blood pressure monitor 720 can correspond to a second gas bladder (e.g., gas bladder 515B), a second set of sensor(s) (e.g., sensor 520B), and/or a second valve (e.g., valve 525B).

In some examples, the first blood pressure monitor 710 and the second blood pressure monitor 720 can synchronize and/or share their blood pressure data (e.g., measurements of diastolic blood pressure and/or systolic blood pressure) with each other and/or with a third system (e.g., a comparator system). The first blood pressure monitor 710, the second blood pressure monitor 720, and/or the third (comparator) system can compare the blood pressure data (e.g., measurements of diastolic blood pressure and/or systolic blood pressure) from the first blood pressure monitor 710 and the second blood pressure monitor 720, and use the compared data for a number of functions. These functions can include calibrating the first blood pressure monitor 710 and/or the second blood pressure monitor 720 (e.g., especially if one is known to be more accurate and/or reliable than the other, for instance where one is a relatively inaccurate portable or wearable device while the other is a more accurate invasive arterial line), determining averaged blood pressure values (e.g., averaged measurements of diastolic blood pressure and/or systolic blood pressure), identifying if one of the blood pressure monitors is inaccurate or producing errors, diagnosing errors in the blood pressure monitors, or a combination thereof.

As illustrated in the graph diagram 700, the measurements for both diastolic blood pressure and systolic blood pressure are higher earlier along the timeline (e.g., closer to a time 705 of zero) and lower later along the timeline (e.g., further away from the time 705 of zero). This can be caused by increased activity before the blood pressure measurement. For instance, walking over to a chair and sitting down in the chair to prepare for the blood pressure measurement can be difficult for some patients, especially those who are elderly, obese, or sick. Such patients can react to exertion with hypertension. Thus, blood pressure values can be elevated early into measurement, especially for such patients. Over time, blood pressure values become asymptotic and stabilize. For instance, blood pressure can stabilize for certain patients in 5 minutes. For certain patients, such as those who are elderly, obese, and/or sick, blood pressure can take longer than 5 minute to stabilize.

Because the physiological attribute monitoring systems disclosed herein (e.g., the physiological attribute monitoring system 100, physiological attribute monitoring system 300, physiological attribute monitoring system 500, and/or physiological attribute monitoring system 600) can provide improved longer-term physiological attribute monitoring (e.g., blood pressure monitoring) without cutting off blood flow, both due to faster blood pressure value detection and the ability to continuously monitor a single blood pressure value (e.g., diastolic blood pressure) (or even both blood pressure values using different gas bladders) over time, blood pressure can more easily be measured for longer durations of time, for instance allowing patients and physicians to quickly obtain accurate blood pressure values for durations of 5 minutes or even longer. The blood pressure monitoring system can use a moving average to determine blood pressure values over time in this way, ensuring the blood pressure is not overestimated. In some examples, the physiological attribute monitoring system can automatically track blood pressure values and output an alert (e.g., via the I/O interface(s) 160) when the blood pressure values have stabilized (e.g., are stable within a predetermined threshold pressure range for at least a predetermined amount of time), so that blood pressure monitoring of the patient 105 is not stopped too early.

It should be understood that the physiological attribute monitoring systems and/or the blood pressure monitoring systems disclosed herein are not limited to monitoring blood pressure, but can also monitor other physiological attributes such as heart rate, respiratory rate, oxygen arterial blood saturation, cardiac output, other physiological attributes disclosed herein, or a combination thereof.

FIG. 8 is a block diagram illustrating a physiological attribute monitoring system 800 with a cuff 110 that is divided (along the circumference of the body part) into multiple gas pillows 830. The physiological attribute monitoring system 800 is an example of the physiological attribute monitoring system 100. The multiple gas pillows 830 are examples of the gas pillow(s) 126. The cuff 110 is divided into gas pillows 830 along the circumference of the body part 505 (e.g., along the circumference of the arm) of the patient 105. For instance, as illustrated in FIG. 8, the cuff 110 is divided into eleven gas pillows 830, but can have more or fewer than eleven in some examples.

In some examples, the pneumatic system 115 can inflate and/or deflate the different gas pillows 830 uniformly. For instance, in some examples, the pneumatic system 115 can inflate the different gas pillows 830 at uniform inflation rates and/or can deflate the different gas pillows 830 at uniform deflation rates. In some examples, when inflating the cuff 110, the pneumatic system 115 can inflate the different gas pillows 830 to the same pressure. In some examples, when deflating the cuff 110, the pneumatic system 115 can deflate the different gas pillows 830 to the same pressure.

In some examples, the pneumatic system 115 can inflate and/or deflate the different gas pillows 830 with variability in rates and/or pressures. For instance, in some examples, the pneumatic system 115 can inflate the different gas pillows 830 at different inflation rates and/or can deflate the different gas pillows 830 at different deflation rates. In some examples, when inflating the cuff 110, the pneumatic system 115 can inflate the different gas pillows 830 to different pressures. In some examples, when deflating the cuff 110, the pneumatic system 115 can deflate the different gas pillows 830 to different pressures.

In some examples, the cuff 110 includes overlapping portions that overlap over each other while the cuff 110 is worn by the patient 105. For instance, if the cuff 110 includes fasteners that fasten one portion of the cuff 110 to another portion of the cuff 110, such as hook-and-loop fasteners (e.g., Velcro® fasteners), magnetic fasteners, or adhesive fasteners-a first portion of the cuff 110 (with a first set of fasteners) generally overlaps over a second portion of the cuff 110 (with a second set of fasteners that fasten to the first set of fasteners). In some examples, the physiological attribute monitoring system 800 can inflate a first subset of the gas pillows 830 that are in one or both of these overlapping portions of the cuff 110 less than second subset of the gas pillows 830 that are not in any of the overlapping portions of the cuff 110. This can prevent the overlapping portion(s) of the cuff 110 from having increased volume I think the stays the same (e.g., even double volume) compared to the non-overlapping portions of the cuff 110, making the cuff 110 more reliable, accurate, and flexible—for instance by allowing the cuff 110 to more accurately accommodate and account for different patients having different sizes of the body part 505.

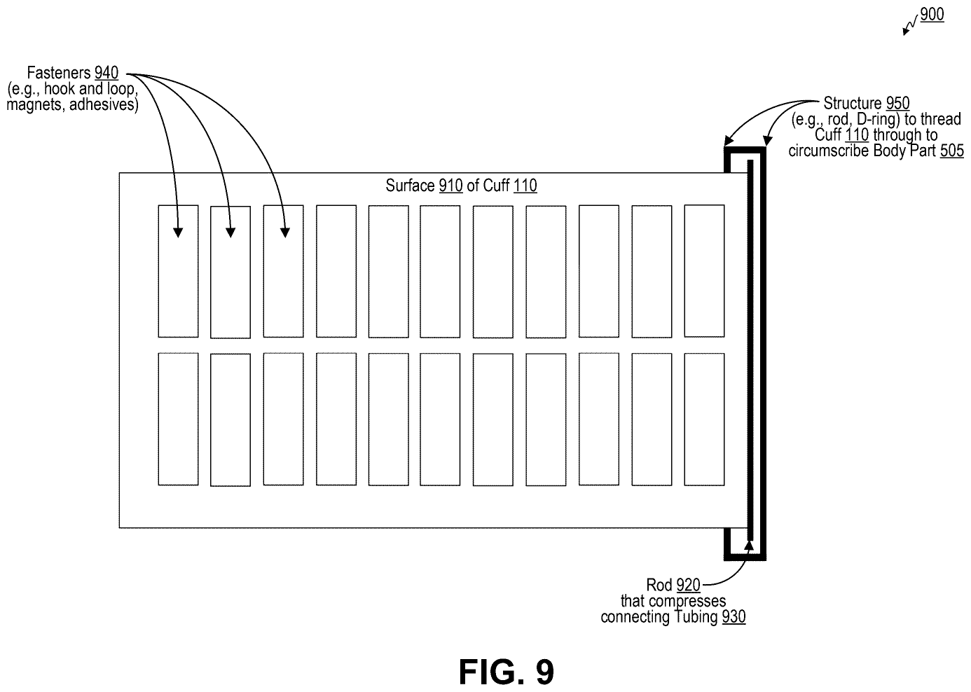

In some examples, the physiological attribute monitoring system 800 can inflate a first subset of the gas pillows 830 that are not in these overlapping portions of the cuff 110, as well as a portion of the second subset that is closer to (more proximal to) the body part 505 of the patient 105. In some examples, a ring, rod, or other structure at an end of cuff 110 (e.g., see rod 920 and/or structure 950 of FIG. 9) can automatically squeeze and/or pinch the tubing 840 between the gas pillows 830 closed, thus closing off the flow of gas to the potion of the second subset (e.g., the overlapping portion) that is further away from (more distal from) the body part 505 of the patient 105, leaving those gas pillows empty or with very little gas. In some examples, the ring, rod, or other structure at the end of cuff 110 (e.g., see rod 920 and/or structure 950 of FIG. 9) has an edge that the tubing 840 bends around. For instance, when the cuff threads through the structure 950 and bend over the rod 920 (or other edge), this can occlude the tubing 840, preventing gas from moving further through the tubing 840 to gas pillows that are beyond the bend in the tubing 840. The tubing 840 can be made of soft, flexible materials to allow the tubing 840 to bend around the rod 920, the structure 950, and/or other edges, rings, or other structures.

In some examples, the gas pillows 830 are coupled to one another using tubing (e.g., tubing 840, which may be examples of the tube(s) 128), so that gas can transfer from one gas pillow to another. For instance, in the context of the physiological attribute monitoring system 800 of FIG. 8, during inflation of the cuff 110, the tubing 810 can deliver gas 815 (e.g., from the pneumatic system 115) to the cuff 110, starting from the rightmost gas pillow of the different gas pillows 830. The gas 815 can be conveyed through the tubing 840 to the different gas pillows 830, for instance from right to left. During deflation of the cuff 110, the cuff 110 can release the gas 815 through the tubing 820. Gas 815 from gas pillows 830 that are farther from the tubing 820 can be first conveyed through the tubing 840, for instance from left to right, before being released through the tubing 820. In some examples, the gas pillows 830 are not directly coupled to one another with any tubing, so there is no gas transferred from one gas pillow to another.

In some examples, physiological attribute monitoring system 800 may represent an entire cuff 110. In some examples, the physiological attribute monitoring system 800 may represent a single gas bladder of a cuff 110, such as one of the gas bladders 515A-515E (or one of the gas bladder(s) 124). In some examples, certain tubes (e.g., the tubing 820) can be discarded or removed, as the gas 815 can be vented from valves directly on the bladder(s) and/or pillow(s), or the same tubing 810 can also receive the gas 815 during deflation. The tubing 840 that connects the gas pillows 830 can include lumens and/or openings between walls or membranes between the gas pillows 830.

FIG. 9 is a block diagram illustrating a physiological attribute monitoring system 900 with a cuff 110 that includes fasteners 940 along a surface 910 of the cuff 110. The surface 910 can be an exterior surface of the cuff 110 (e.g., that faces away from the body part 505 of the patient 105 when the patient 105 wears the cuff 110 on the body part 505), an interior surface of the cuff 110 (e.g., that faces toward from the body part 505 of the patient 105 when the patient 105 wears the cuff 110 on the body part 505), or a combination thereof.

The fasteners 940 can each include, for instance, hook and loop fasteners (e.g., Velcro® fasteners), magnetic fasteners (e.g., magnets, ferromagnetic materials, ferromagnets, electromagnets, metals, rare earth minerals, or combinations thereof), clips, pins, hooks, loops, screws, threads, elastics, ties, adhesives (e.g., glues, tapes, and/or reusable adhesives), or combinations thereof. In some examples, the physiological attribute monitoring system 900 can include sensors (e.g., sensor(s) 134, sensor(s) 148, sensor(s) 152, sensors 520A-520E) that can obtain measurements that a monitoring system 120 of the physiological attribute monitoring system 900 can use to determine which first fastener of the fasteners 940 is attached which second fastener of the fasteners 940, which the monitoring system 120 of the physiological attribute monitoring system 900 can use in turn to determine the size (e.g., circumference) of the body part 505 of the patient 105.

For instance, in some examples, the fasteners 940 include a first fastener with an electromagnet and a second fastener with a ferromagnetic material (e.g., a magnet or a material that is ferromagnetic). When the cuff 110 is secured around the body part 505 of the patient 105, the first fastener (the electromagnet) magnetically couples to the second fastener (the ferromagnetic material). In some examples, the monitoring system 120 of the physiological attribute monitoring system 900 can identify that the first fastener (the electromagnet) is in use to secure the cuff 110 to the body part 505 of the patient 105 based on detection (e.g., using the sensor(s)) of an electrical attribute (e.g., voltage, resistance, current, or a combination thereof) corresponding to the first fastener (the electromagnet) in the second fastener (the ferromagnetic material) (e.g., a voltage from the electromagnet running through the ferromagnetic material). In some examples, the electromagnet(s) cane activated and/or deactivated in response to receipt of an input through an interactive user interface (e.g., a button pressed by a user), can be activated and/or deactivated automatically by the physiological attribute monitoring system 900 system, or a combination thereof.

In some examples, the fasteners 940 include multiple electromagnets that each have different electrical attributes (e.g., voltage, resistance, current, or a combination thereof). When the cuff 110 is secured around the body part 505 of the patient 105, the monitoring system 120 of the physiological attribute monitoring system 900 can identify that a specific fastener (a specific electromagnet) is in use to secure the cuff 110 to the body part 505 of the patient 105 based on detection (e.g., using the sensor(s)) of an electrical attribute (e.g., voltage, resistance, current, or a combination thereof) corresponding to that specific fastener (that specific electromagnet) on another fastener (e.g., another electromagnet or ferromagnetic material). These can be used for AI/ML system (e.g., artificial intelligence system 1000) and to estimate body part (e.g., arm) circumference. In some examples, the electromagnet(s) cane activated and/or deactivated in response to receipt of an input through an interactive user interface (e.g., a button pressed by a user), can be activated and/or deactivated automatically by the physiological attribute monitoring system 900 system, or a combination thereof.

In some examples, the sensor(s) (e.g., sensor(s) 134, sensor(s) 148, sensor(s) 152, sensors 520A-520E) can include pressure sensors, optical sensors, or other sensors that the physiological attribute monitoring system 900 can use to detect an overlapping portion of the cuff 110, to detect the size (e.g., circumference) of the body part 505 of the patient 105.

The physiological attribute monitoring system 900 also illustrates a rod 920 and a structure 950. The structure 950 can include a second rod, a D-ring, or a combination thereof. In some examples, the cuff 110 is threaded through the structure 950 (and/or alongside the rod 920) to circumscribe the body part 505 of the patient 105. In some examples, the rod 920 and/or the structure 950 can compress connecting tubing 840, such as the tubing 840 between gas pillows 830, so that certain gas pillows (e.g., in overlapping portion(s) of the cuff 110) do not receive gas, or receive less gas, than other gas pillows (e.g., in non-overlapping portion(s) of the cuff 110). For instance, when the gas bladder(s) of the cuff 110 go through the structure 950 and/or alongside the rod 920, causing the 110 to bend, this bend can cause automatic adjustment of the used part of the bladder (e.g., inflated or deflated) to the arm circumference.

In some examples, when the cuff 110 is threaded through the through the structure 950 and/or alongside the rod 920, the functional divisions (e.g., the tubing 840) between the sections of each bladder (e.g., the different gas pillows of each bladder) become impermissible (“closed”) for flow of the gas. This can allow for automatic adjustment of the inflated bladder length to the circumference of body part 505. This also allows to use minimal volume of gas (e.g., to preserve batteries, to preserve gas from the compressed gas canister 350, and the like).

FIG. 10 is a block diagram illustrating an example of an artificial intelligence system 1000 for training, use of, and/or updating of one or more machine learning model(s) 1025 that are used to generate physiological attribute curve(s) 1035, physiological attribute value(s) 1040, and/or recommendation(s) 1042. The machine learning (ML) system 1000 includes an ML engine 1020 that generates, trains, uses, and/or updates one or more ML model(s) 1025. In some examples, the AI algorithm(s) 158 includes the ML system 1000, the ML engine 1020, the ML model(s) 1025, and/or the feedback engine(s) 1050, or vice versa.

The ML model(s) 1025 can include, for instance, one or more neural network(s) (NN(s)), convolutional NN(s) (CNN(s)), time delay NN(s) (TDNN(s)), deep network(s) (DN(s)), autoencoder(s) (AE(s)), variational autoencoder(s) (VAE(s)), deep belief net(s) (DBN(s)), recurrent NN(s) (RNN(s)), generative adversarial network(s) (GAN(s)), conditional GAN(s) (cGAN(s)), feed-forward network(s), network(s) having fully connected layers, support vector machine(s) (SVM(s)), random forest(s) (RF), computer vision (CV) system(s), autoregressive (AR) model(s), Sequence-to-Sequence (Seq2Seq) model(s), large language model(s) (LLM(s)), multimodal large language model(s) (MLLM(s)), deep learning system(s), classifier(s), transformer(s), gradient boosted model(s), gradient boosted tree(s), regression model(s), autoregression model(s), or any other supervised on unsupervised ML or AI model, or a combination thereof.

In some examples, the ML model(s) 1025 can include a U-Network (U-Net) structure and/or architecture that includes a contracting path and an expansive path. If the ML model(s) 1025 is a U-Net, the ML model(s) 1025 may include, for instance, combination of convolution, up-convolution, pooling and skip connections that allows the ML model(s) 1025 to extract and capture complex features, while also keeping and reconstructing spatial information.

In examples where the ML model(s) 1025 include LLMs and/or MLLMs, the LLMs and/or MLLMs can include, for instance, a Generative Pre-Trained Transformer (GPT) (e.g., GPT-2, GPT-3, GPT-3.5, GPT-4, etc.), DaVinci or a variant thereof, an LLM using Massachusetts Institute of Technology (MIT)® langchain, Pathways Language Model (PaLM), Large Language Model Meta® AI (LLaMA), Language Model for Dialogue Applications (LaMDA), Google® Gemini®, Anthropic® Claude®, Anthropic® Claude® Sonnet®, Bidirectional Encoder Representations from Transformers (BERT), Anthropic® Claude®, Falcon (e.g., 40B, 7B, 1B), Orca, Phi-1, StableLM, DeepSeek® R1, Alibaba® Qwen®, ByteDance® Doubao®, another LLM or MLLM, variant(s) of any of the previously-listed LLMs or MLLMs, or a combination thereof.

Within FIG. 10, a graphic representing the ML model(s) 1025 illustrates a set of circles connected to one another. Each of the circles can represent a node, a neuron, a perceptron, a layer, a portion thereof, or a combination thereof. The circles are arranged in columns. The leftmost column of white circles represent an input layer. The rightmost column of white circles represent an output layer. Two columns of shaded circled between the leftmost column of white circles and the rightmost column of white circles each represent hidden layers. An ML model can include more or fewer hidden layers than the two illustrated, but includes at least one hidden layer. In some examples, the layers and/or nodes represent interconnected filters, and information associated with the filters is shared among the different layers with each layer retaining information as the information is processed. The lines between nodes can represent node-to-node interconnections along which information is shared. The lines between nodes can also represent weights (e.g., numeric weights) between nodes, which can be tuned, updated, added, and/or removed as the ML model(s) 1025 are trained and/or updated. In some cases, certain nodes (e.g., nodes of a hidden layer) can transform the information of each input node by applying activation functions (e.g., filters) to this information, for instance applying convolutional functions, downscaling, upscaling, data transformation, and/or any other suitable functions.

In some examples, the ML model(s) 1025 can include a feed-forward network, in which case there are no feedback connections where outputs of the network are fed back into itself. In some cases, the ML model(s) 1025 can include a recurrent neural network, which can have loops that allow information to be carried across nodes while reading in input. In some cases, the network can include a convolutional neural network, which may not link every node in one layer to every other node in the next layer.

One or more input(s) 1005 can be provided to the ML model(s) 1025. The ML model(s) 1025 can be trained by the ML engine 1020 (e.g., based on training data 1065) to generate one or more output(s) 1030. In some examples, the input(s) 1005 include information 1010. The information 1010 can include, for instance, sensor data from sensors (e.g., sensor(s) 134, sensor(s) 148, sensor(s) 152, sensors 520A-520E), patient age, patient sex, patient gender, patient weight, patient height, patient size (e.g., circumference of a body part 505), patient ethnicity, patient medical record, patient medical history, patient family member medical records, patient family member medical histories, symptoms experienced by the patient, diseases that the patient suffers from, records of past physiological attributes (e.g., blood pressures, heart rates, etc.) of the patient and/or other patients (e.g., similar patients with shared characteristics), other information about the patient and/or similar patients with shared characteristics, information from medical textbooks and/or medical journals, other medical context, symptoms entered through an interactive user interface of the monitoring system 120 (e.g., through the I/O interface(s) 160), mood of the patient 105, weather, atmospheric pressure, height of the physiological attribute monitoring system 100 (e.g., the monitoring system 120) above the sea level, atmospheric temperature, temperature of the gas in tubing or portion(s) (e.g., gas pillow(s) 126, gas bladder(s) 124) of the cuff 110 and/or other systems (e.g., central pressure system, gas canisters), pressure in gas canisters, pressure in central pressure system delivering gas to or through the pneumatic system 115 to the cuff 110, characteristics of the patient 105, characteristics of another patient with shared characteristic(s) with the patient 105, data associated with another patient using the same type of physiological attribute monitoring system 100, or a combination thereof. In some examples, the input(s) 1005 can include prompt(s) (e.g., to an LLM). In some examples, the input(s) 1005 can include information retrieved from data store(s) 1070, for instance via retrieval augmented generation (RAG) (e.g., via RAG query(s) 1045). In some examples, the input(s) 1005 can include prompt(s) that are modified and/or enhanced using information retrieved from data store(s) 1070, for instance via retrieval augmented generation (RAG) (e.g., via RAG query(s) 1045).

The output(s) 1030 that ML model(s) 1025 generate by processing the input(s) 1005 (e.g., the information 1010 and/or the previous output(s) 1015) can include physiological attribute curve(s) 1035, physiological attribute value(s) 1040, recommendation(s) 1042, and/or RAG query(s) 1045. The physiological attribute curve(s) 1035 can include, for instance, curves that relate a given physiological attribute (e.g., blood pressure, heart rate, respiratory rate, cardiac output, or other physiological attributes discussed herein) to cuff pressure (e.g., pressure in the cuff 110, in gas bladder(s) 124, and/or in gas pillow(s) 126), curves that relate gas pressure to time, curves that relate the physiological attribute to a blood pressure wave signal amplitude, amplitude thresholds for blood pressure wave signals, curves that relate the amplitude thresholds to cuff pressure, or combinations thereof. In an illustrative example, the physiological attribute curve(s) 1035 relate pressure (e.g., in the cuff 110, in at least one of the gas bladder(s) 124, in at least one of the gas pillow(s) 126, and/or in the body part 505 of the patient 105) to time. In some examples, the physiological attribute curve(s) 1035 can vary from patient to patient, especially, for patients who are different sizes (e.g., different circumferences of body part 505), so using the ML model(s) 1025 to determine the physiological attribute curve(s) 1035 automatically can improve accuracy and/or expedite determination of physiological attribute values (e.g., blood pressure values, heart rate values, respiratory rate values, cardiac output values, or values for other physiological attributes discussed herein) for a patient. In some examples, the ML model(s) 1025 determine a shape of a physiological attribute curve of the physiological attribute curve(s) 1035.

The physiological attribute value(s) 1040 can include, for instance, systolic blood pressure, diastolic blood pressure, other blood pressure metrics or values, heart rate values, respiratory rate values, cardiac output values, or values for other physiological attributes discussed herein, or a combination thereof. In some examples, the ML model(s) 1025 determine the physiological attribute value(s) 1040 based on the physiological attribute curve(s) 1035 (e.g., based on a shape of a physiological attribute curve of the physiological attribute curve(s) 1035).

The recommendation(s) 1042 can include, for instance, a diagnosis (e.g., of a disease related to hypertension or hypotension), a recommendation to prescribe a specific medication (e.g., for hypertension or hypotension), a recommendation to perform a specific procedure (e.g., for hypertension or hypotension), a recommendation to provide a specific treatment (e.g., for hypertension or hypotension), a recommendation to obtain more information (e.g., from the patient 105), a recommendation to perform a specific test or scan (e.g., Magnetic resonance imaging (MRI), positron emission tomography (PET) scan, computed axial tomography (CAT) scan, X-ray, ultrasound, angiography)), a recommendation to re-test blood pressure (e.g., due to higher than threshold likelihood of error or inaccuracy), a recommendation to see a provider, a recommendation to contact a provider, a recommendation to recheck blood pressure or other bodily measurable characteristics, a recommendation for the patient to stay still for the time of measurement, a recommendation to replace batteries, a recommendation to take or give a medicine to the patient, a recommendation to withhold medicine from the patient, a recommendation for the patient to drink more water, a recommendation for the patient to eat more high potassium foods, a recommendation for the patient to eat less high potassium foods, a recommendation for the patient to make another change to their diet, a recommendation for the patient to go for a walk, a recommendation for the patient to exercise more, recommendation for the patient to rest, a recommendation for the patient to wake up, a recommendation for the patient to sleep, a recommendation for the patient to get more sleep more regularly, a recommendation for the patient to make another type of change to their lifestyle, a recommendation for the physician to obtain blood test for the patient, a recommendation for the physician to obtain a urine test for the patient, a recommendation to replace the gas canister (e.g., compressed gas canister(s) 142, compressed gas canister 350), or a combination thereof. The ML model(s) 1025 can generate each of the output(s) 1030 based on the information 1010, information from the data store(s) 1070, and/or other types of input(s) 1005 (e.g., previous output(s) 1015).

In some examples, the ML model(s) 1025 can identify something in the input(s) 1005 about which the data store(s) 1070 include additional information, and can fashion at least one query (e.g., the RAG query(s) 1045) for the data store(s) 1070 to retrieve the additional information from the data store(s) 1070. For instance, if the information 1010 references a specific model of device, the RAG query(s) 1045 can include one or more queries of the data store(s) 1070 for additional information about the specific model of device, for instance to retrieve its components, configurations, settings, firmware updates, ranges of optimal operating parameters (e.g., temperature, clock speed, and so forth), or a combination thereof. The additional information retrieved from the data store(s) 1070 using the RAG query(s) 1045 can be used as part of the input(s) 1005 (e.g., as part of the information 1010 and/or part of the previous output(s) 1015) for further passes of data processing by the ML model(s) 1025.

In some examples, certain output(s) 1030 (e.g., the physiological attribute curve(s) 1035, the physiological attribute value(s) 1040, the recommendation(s) 1042, the RAG query(s) 1045) can be used as part of the input(s) 1005 to the ML model(s) 1025 (e.g., as part of previous output(s) 1015) for identifying other output(s) 1030 (e.g., the physiological attribute curve(s) 1035, the physiological attribute value(s) 1040, the recommendation(s) 1042, the RAG query(s) 1045). For instance, in an illustrative example, the physiological attribute curve(s) 1035 can be processed, as previous output(s) 1015, by the ML model(s) 1025 to generate the physiological attribute value(s) 1040, the recommendation(s) 1042, the RAG query(s) 1045, and/or other output(s) 1030. In some examples, at least some of the previous output(s) 1015 in the input(s) 1005 represent previously-identified instances of some of the output(s) 1030 that are input into the ML model(s) 1025 to generate other types of the output(s) 1030. In some examples, based on receipt of the input(s) 1005, the ML model(s) 1025 can select the output(s) 1030 from a list of possible outputs, for instance by ranking the list of possible outputs by likelihood, probability, and/or confidence based on the input(s) 1005. In some examples, based on receipt of the input(s) 1005, the ML model(s) 1025 can identify the output(s) 1030 at least in part using generative artificial intelligence (AI) content generation techniques, for instance using an LLM to generate custom text and/or graphics identifying the output(s) 1030. In some examples, the LLM-based output(s) 1030 are conversationally responsive to a prompt in the input(s) 1005 (e.g., in the information 1010 and/or in the previous output(s) 1015).

In some examples, the 1025 can generate intermediate data based on the 1005, and the 1025 can generate the output(s) 1030 based on the intermediate data. For instance, in some examples, the ML model(s) 1025 can process the information 1010 to generate a description of a user, to identify behaviors (or categories of behaviors) of the user, to identify an intent of the user, to identify short-term goals of the user, to identify long-term goals of the user, to categorize the user into one of a set of categories (e.g., by behavior, intent, demographics, goals, or a combination thereof), or a combination thereof. The ML model(s) 1025 can then generate the output(s) 1030 based on this intermediate data, for instance to improve customization and/or personalization of the output(s) 1030 to user(s).

In some examples, the ML system repeats the process illustrated in FIG. 10 multiple times to generate the output(s) 1030 in multiple passes, using some of the output(s) 1030 from earlier passes as some of the input(s) 1005 in later passes (e.g., as some of the previous output(s) 1015). For instance, in a first illustrative example, in a first pass, the ML model(s) 1025 can identify the physiological attribute curve(s) 1035 based on input of the information 1010 into the ML model(s) 1025. In a second pass, the ML model(s) 1025 can identify the physiological attribute value(s) 1040 based on input of the information 1010 and the previous output(s) 1015 (that includes the physiological attribute curve(s) 1035 from the first pass) into the ML model(s) 1025. In a third pass, the ML model(s) 1025 can identify the recommendation(s) 1042 based on input of the information 1010 and the previous output(s) 1015 (that includes the physiological attribute curve(s) 1035 from the first pass and/or the physiological attribute value(s) 1040 from the second pass) into the ML model(s) 1025.

In some examples, the ML system includes one or more feedback engine(s) 1050 that generate and/or provide feedback 1055 about the output(s) 1030. In some examples, the feedback 1055 indicates how well the output(s) 1030 align to corresponding expected output(s), how well the output(s) 1030 serve their intended purpose, or a combination thereof. In some examples, the feedback engine(s) 1050 include loss function(s), reward model(s) (e.g., other ML model(s) that are used to physiological attribute curve the output(s) 1030), discriminator(s), error function(s) (e.g., in back-propagation), user interface feedback received via a user interface from a user, or a combination thereof. In some examples, the feedback 1055 can include one or more alignment physiological attribute curve(s) that physiological attribute curve a level of alignment between the output(s) 1030 and the expected output(s) and/or intended purpose.

The ML engine 1020 of the ML system can update (e.g., further train and/or fine-tune) the ML model(s) 1025 based on the feedback 1055 to perform an update 1060 (e.g., further training and/or fine-tuning) of the ML model(s) 1025 based on the feedback 1055. In some examples, the feedback 1055 includes positive feedback, for instance indicating that the output(s) 1030 closely align with expected output(s) and/or that the output(s) 1030 serve their intended purpose. In some examples, the feedback 1055 includes negative feedback, for instance indicating a mismatch between the output(s) 1030 and the expected output(s), and/or that the output(s) 1030 do not serve their intended purpose. For instance, high amounts of loss and/or error (e.g., exceeding a threshold) can be interpreted as negative feedback, while low amounts of loss and/or error (e.g., less than a threshold) can be interpreted as positive feedback. Similarly, high amounts of alignment (e.g., exceeding a threshold) can be interpreted as positive feedback, while low amounts of alignment (e.g., less than a threshold) can be interpreted as negative feedback.

In response to positive feedback in the feedback 1055, the ML engine 1020 can perform the update 1060 to update the ML model(s) 1025 to strengthen and/or reinforce weights (and/or connections and/or hyperparameters) associated with generation of the output(s) 1030 to encourage the ML engine 1020 to generate similar output(s) 1030 given similar input(s) 1005. In this way, the update 1060 can improve the ML model(s) 1025 itself by improving the accuracy of the ML model(s) 1025 in generating output(s) 1030 that are similarly accurate given similar input(s) 1005. In response to negative feedback in the feedback 1055, the ML engine 1020 can perform the update 1060 to update the ML model(s) 1025 to weaken and/or remove weights (and/or connections and/or hyperparameters) associated with generation of the output(s) 1030 to discourage the ML engine 1020 from generating similar output(s) 1030 given similar input(s) 1005. In this way, the update 1060 can improve the ML model(s) 1025 itself by improving the accuracy of the ML model(s) 1025 in generating output(s) 1030 are more accurate given similar input(s) 1005. In some examples, for instance, the update 1060 can improve the accuracy of the ML model(s) 1025 in generating output(s) 1030 by reducing false positive(s) and/or false negative(s) in the output(s) 1030.

For instance, here, if the physiological attribute curve(s) 1035, the physiological attribute value(s) 1040, and/or the recommendation(s) 1042 are used for a subsequent process (e.g., a physiological attribute value determination, a diagnosis, and/or a medication prescription), and the subsequent process is successful (e.g., the physiological attribute value determination and/or diagnosis is accurate, and/or the medication works to relieve the patient's symptoms), the success of the subsequent process can be interpreted as feedback 1055 that is positive (e.g., positive feedback). On the other hand, if the physiological attribute curve(s) 1035 and/or physiological attribute value(s) 1040 are used in a subsequent process (e.g., a physiological attribute value determination, a diagnosis, and/or a medication prescription), and the subsequent process fails or is unsuccessful (e.g., the physiological attribute value determination and/or the diagnosis is inaccurate, and/or the medication fails to relieve the patient's symptoms), the failure or lack of success of the subsequent process can be interpreted as feedback 1055 that is negative (e.g., negative feedback). Either way, the update 1060 can improve the artificial intelligence system 1000 and the overall system by improving the consistency with which the ML model(s) 1025 are successful, and improving the consistency with which the subsequent process (e.g., physiological attribute value determination, diagnosis, and/or medication prescription) is successful.

In some examples, the ML engine 1020 can also perform an initial training of the ML model(s) 1025 before the ML model(s) 1025 are used to generate the output(s) 1030 based on the input(s) 1005. During the initial training, the ML engine 1020 can train the ML model(s) 1025 based on training data 1065. In some examples, the training data 1065 includes examples of input(s) (of any input types discussed with respect to the input(s) 1005), output(s) (of any output types discussed with respect to the output(s) 1030), and/or feedback (of any feedback types discussed with respect to the feedback 1055). In some cases, positive feedback in the training data 1065 can be used to perform positive training, to encourage the ML model(s) 1025 to generate output(s) similar to the output(s) in the training data given input of the corresponding input(s) in the training data. In some cases, negative feedback in the training data 1065 can be used to perform negative training, to discourage the ML model(s) 1025 from generating output(s) similar to the output(s) in the training data given input of the corresponding input(s) in the training data. In some examples, the training of the ML model(s) 1025 (e.g., the initial training with the training data 1065, update(s) 1060 based on the feedback 1055, and/or other modification(s)) can include fine-tuning of the ML model(s) 1025, retraining of the ML model(s) 1025, or a combination thereof.

In some examples, the ML model(s) 1025 can include an ensemble of multiple ML models, and the ML engine 1020 can curate and manage the ML model(s) 1025 in the ensemble. The ensemble can include ML model(s) 1025 that are different from one another to produce different respective outputs, which the ML engine 1020 can average (e.g., mean, median, and/or mode) to identify the output(s) 1030. In some examples, the ML engine 1020 can calculate the standard deviation of the respective outputs of the different ML model(s) 1025 in the ensemble to identify a level of confidence in the output(s) 1030. In some examples, the standard deviation can have an inverse relationship with confidence. For instance, if the respective outputs of the different ML model(s) 1025 are very different from one another (and thus have a high standard deviation above a threshold), the confidence that the output(s) 1030 are accurate may be low (e.g., below a threshold). On the other hand, if the respective outputs of the different ML model(s) 1025 are equal or very similar to one another (and thus have a low standard deviation below a threshold), the confidence that the output(s) 1030 are accurate may be high (e.g., above a threshold).

In some examples, different ML models(s) 1025 in the ensemble can include different types of models. For instance, in some examples, an ensemble can include a NN and a SVM that are both trained to process the input(s) 1005 to generate at least a subset of the output(s) 1030. In some examples, the ensemble may include different ML model(s) 1025 that are trained to process different inputs of the input(s) 1005 and/or to generate different outputs of the output(s) 1030. For instance, in some examples, a first model (or set of models) can process the input(s) 1005 to generate the physiological attribute curve(s) 1035, a second model (or set of models) can process the input(s) 1005 to generate the physiological attribute value(s) 1040, a third model (or set of models) can process the input(s) 1005 to generate the recommendation(s) 1042, and a fourth model (or set of models) can process the input(s) 1005 to generate the RAG query(s) 1045. In some examples, the ML engine 1020 can choose specific ML model(s) 1025 to be included in the ensemble because the chosen ML model(s) 1025 are effective at accurately processing particular types of input(s) 1005, are effective at accurately generating particular types of output(s) 1030, are generally accurate, process input(s) 1005 quickly, generate output(s) 1030 quickly, are computationally efficient, have higher or lower degrees of uncertainty than other models in the ensemble, or a combination thereof.

In some examples, one or more of the ML model(s) 1025 can be initialized with weights, connections, and/or hyperparameters that are selected randomly. This can be referred to as random initialization. These weights, connections, and/or hyperparameters are modified over time through training (e.g., initial training with the training data 1065 and/or update(s) 1060 based on the feedback 1055), but the random initialization can still influence the way the ML model(s) 1025 process data, and thus can still cause different ML model(s) 1025 (with different random initializations) to produce different output(s) 1030. Thus, in some examples, different ML model(s) 1025 in an ensemble can have different random initializations.

In some examples, the data collected and available during at least one or more subsequent time units (e.g., continuously subsequent time units or not continuously subsequent time units) of the operation of the physiological attribute monitoring system 100 can be used (1) to train, retrain, or tune of ML model(s) 1025, (2) to obtain output(s) 1030 (e.g., physiological attribute curve(s) 1035, physiological attribute value(s) 1040, recommendation(s) 1042), and/or (3) to modify the operation (e.g., switching off or on, restarting, changing settings) of the physiological attribute monitoring system 100 (e.g., of the cuff 110, the pneumatic system 115, and/or the monitoring system 120), for next one or more time units (e.g., continuously subsequent time units or not continuously subsequent time units).

As an ML model (of the ML model(s) 1025) is trained (e.g., along the initial training with the training data 1065, update(s) 1060 based on the feedback 1055, and/or other modification(s)), different versions of the ML model at different stages of training can be referred to as checkpoints. In some examples, after each new update to a model (e.g., update 1060) generates a new checkpoint for the model, the ML engine 1020 tests the new checkpoint (e.g., against testing data and/or validation data where the correct output(s) are known) to identify whether the new checkpoint improves over older checkpoints or not, and/or if the new checkpoint introduces new errors (e.g., false positive(s) and/or false negative(s)). This testing can be referred to as checkpoint benchmark scoring. In some examples, in checkpoint benchmark scoring, the ML engine 1020 produces a benchmark physiological attribute curve for one or more checkpoint(s) of one or more ML model(s) 1025, and keeps the checkpoint(s) that have the best (e.g., highest or lowest) benchmark physiological attribute curves in the ensemble. In some examples, if a new checkpoint is worse than an older checkpoint, the ML engine 1020 can revert to the older checkpoint. The benchmark physiological attribute curve for a can represent a level of accuracy of the checkpoint and/or number of errors (e.g., false positive or false negative) by the checkpoint during the testing (e.g., against the testing data and/or the validation data). In some examples, an ensemble of the ML model(s) 1025 can include multiple checkpoints of the same ML model.

In some examples, the ML model(s) 1025 can be trained and/or updated (e.g., with training data 1065 and/or the update 1060) over a number of epochs, where each epoch includes one or more batches of training data. A batch refers to a subset of the training data that is processed together in a single forward and backward pass during model training. Processing data in batches can improve computational efficiency and stabilize gradient updates. As training progresses across multiple epochs, the model parameters are iteratively refined, enabling the model to generalize better to unseen data. The use of multiple epochs and batch-based updates allows the model to progressively learn complex patterns and reduce prediction error, thereby improving accuracy and/or robustness of the ML model(s) 1025 over time. The number of epochs and batch size may be predetermined or dynamically adjusted based on performance of the ML model(s) 1025, convergence criteria, and/or available computational resources.

In some examples, the ML model(s) 1025 can be modified, either through the initial training (with the training data 1065), an update 1060 based on the feedback 1055, or another modification to introduce randomness, variability, and/or uncertainty into an ensemble of the ML model(s) 1025. In some examples, such modification(s) to the ML model(s) 1025 can include dropout (e.g., Monte Carlo dropout), in which one or more weights or connections are selected at random and removed. In some examples, dropout can also be performed during inference, for instance to modify the output(s) 1030 generated by the ML model(s) 1025. The term Bayesian Machine Learning (BML) can refer to random dropout, random initialization, and/or other randomization-based modifications to the ML model(s) 1025. In some examples, the modification(s) to the ML model(s) 1025 can include a hyperparameter search and/or adjustment of hyperparameters. The hyperparameter search can involve training and/or updating different ML models 1025 with different values for hyperparameters and evaluating the relative performance of the ML models 1025 (e.g., against testing data and/or validation data where the correct output(s) are known) to identify which of the ML models 1025 performs best. Hyperparameters can include, for instance, temperature (e.g., influencing level creativity and/or randomness), top P (e.g., influencing level creativity and/or randomness), frequency penalty (e.g., to prevent repetitive language between one of the output(s) 1030 and another), presence penalty (e.g., to encourage the ML model(s) 1025 to introduce new data in the output(s) 1030), other parameters or settings, or a combination thereof.

In some examples, the ML engine 1020 can perform retrieval-augmented generation (RAG) using the model(s) 1025. For instance, in some examples, the ML engine 1020 can pre-process the input(s) 1005 by retrieving additional information from one or more data store(s) 1070 (e.g., any of the databases and/or other data structures discussed herein) and using the additional information to enhance the input(s) 1005 before the input(s) 1005 are processed by the ML model(s) 1025 to generate the output(s) 1030. For instance, in some examples, the enhanced versions of the input(s) 1005 can include the additional information that the ML engine 1020 retrieved from the one or more data store(s) 1070. In some examples, the artificial intelligence system 1000 can retrieve the additional information from one or more data store(s) 1070 by querying the data store(s) 1070 using RAG query(s) 1045 generated by the ML model(s) 1025 (or extracted from the input(s) 1005 using the ML model(s) 1025). In some examples, this RAG process provides the ML model(s) 1025 with more relevant information, allowing the ML model(s) 1025 to generate more accurate and/or personalized output(s) 1030.

In some examples, the analyses and data processing operations discussed as being performed by the ML model(s) 1025 can be performed by different types of AI algorithm(s) 158 (other than ML model(s)) in addition to, or instead of, being performed by the ML model(s) 1025. For instance, in some examples, AI algorithm(s) 158 can be used to process the input(s) 1005 (e.g., the information 1010 and/or the previous output(s) 1015) to generate the output(s) 1030 (e.g., the physiological attribute curve(s) 1035, the physiological attribute value(s) 1040, the recommendation(s) 1042, and/or the query(s) 1045). Examples of AI algorithm(s) 158 that the 1000 can use in this way can include rule-based AI systems (e.g., with pre-defined rules), expert-based AI systems (e.g., where experts define the rules), heuristic-based AI systems (e.g., where various key values are combined using a specific equation or formula to generate a heuristic that the systems compare to a heuristic threshold or to other generated heuristics to make a decision), genetic AI algorithms (e.g., that evolve solutions through iterative processes but without learning), search algorithms (e.g., that explore a search space to find optimal solutions to problems), symbolic AI algorithms (e.g., that focus on representing knowledge and reasoning using symbols and logical rules), or combinations thereof.

FIG. 11 is a block diagram illustrating a RAG system that may be used to implement some aspects of the technology. The RAG system 1100 includes one or more interface device(s) 1110 that can receive input(s) from a user and/or a user device, for instance by receiving a query 1130 and/or a prompt 1135 from the user and/or the system. The input(s) 1005 can be an example of the prompt 1135. The query 1130 can be an example of the RAG query(s) 1045 and/or a sub-query about a specific term in the input(s) 1005 (e.g., in the prompt 1135).

The interface device(s) 1110 can send the query 1130 to one or more data store system(s) 1115 that include, and/or that have access to (e.g., over a network connection), various data store(s) (e.g., database(s), table(s), spreadsheet(s), tree(s), ledger(s), heap(s), and/or other data structure(s)). The data store system(s) 1115 searches the data store(s) according to the query 1130. In some examples, the interface device(s) 1110 and/or the system(s) 1115 convert the query 1130 into tensor format (e.g., vector format and/or matrix format). In some examples, the data store system(s) 1115 searches the data store(s) according to the query 1130 by matching the query 1130 with data in tensor format (e.g., vector format and/or matrix format) stored in the data store(s) that are accessible to the data store system(s) 1115. The data store system(s) 1115 retrieve, from the data store(s) and based on the query 1130, information 1140 that is relevant to generating enhanced content 1145.

In some examples, the data store system(s) 1115 provide the information 1140 and/or the enhanced content 1145 to the interface device(s) 1110. In some examples, the data store system(s) 1115 provide the information 1140 to the interface device(s) 1110, and the interface device(s) 1110 generate the enhanced content 1145 based on the information 1140. The interface device(s) 1110 provides the query 1130, the prompt 1135, the information 1140, the enhanced content 1145, and/or enhanced prompt 1150 based on prompt 1135 and enhanced content 1145 to one or more LLM(s) 1125 (e.g., ML model(s) 1025) of an LLM engine 1120 (e.g., ML engine 1020). The LLM(s) 1125 generate response(s) 1155 that are responsive to the prompt 1135. In some examples, the response(s) 1155 may be, or may include, details and/or additional details of an object that the query is based on.

In some examples, the LLM(s) 1125 generate the response(s) 1155 (e.g., including the details of an object) based on the query 1130, the prompt 1135, the information 1140, the enhanced content 1145, and/or the enhanced prompt 1150. In some examples, the LLM(s) 1125 generate the response(s) 1155 to include, or be based on, the information 1140 and/or the enhanced content 1145. The LLM(s) 1125 provides the response(s) 1155 to the interface device(s) 1110. In some examples, the interface device(s) 1110 output the response(s) 1155 to the user (e.g., to the user device of the user) that provided the query 1130 and/or the prompt 1135. In some examples, the interface device(s) 1110 output the response(s) 1155 to the system (e.g., the other ML model) that provided the query 1130 and/or the prompt 1135 to the interface device(s) 1110. In some examples, the data store system(s) 1115 may include one or more ML model(s) that are trained to perform the search of the data store(s) based on the query 1130.

In some examples, the system(s) 1115 provides the information 1140 and/or the enhanced content 1145 directly to the LLM(s) 1125, and the interface device(s) 1110 provide the query 1130 and/or the prompt 1135 to the LLM(s) 1125. The LLM engine 1120 may be an example of the ML engine 1020, or vice versa. The LLM(s) 1125 may be example(s) of the AI algorithm(s) 158, and/or the ML model(s) 1025, or vice versa.

The data store system(s) 1115 can output this information 1140 to the interface device(s) 1110 and/or the LLM engine 1120. The data store system(s) 1115, the interface device(s) 1110, and/or the LLM engine 1120 can modify the prompt 1135 to add the enhanced content 1145 and thereby generate the enhanced prompt 1150. In some examples, the data store system(s) 1115, the interface device(s) 1110, and/or the LLM engine 1120 adds or appends the information 1140 and/or enhanced content 1145 to the prompt 1135 and/or the query 1130 to generate the enhanced prompt 1150. The data store system(s) 1115, device(s) 1110, and/or LLM engine 1120 process the enhanced prompt 1150 using the LLM(s) 1125 to generate the response(s) 1155. The response(s) 1155 can be an example of the output data 195. In some cases, the response(s) 1155 can include visualizations, such as graphs or charts.

In some examples, the LLM engine 1120 also updates (e.g., fine-tunes, adjusts parameters or hyperparameters of, and/or further trains) the LLM(s) 1125 based on the enhanced prompt 1150 (e.g., based on the enhanced content 1145), based on the information 1140, based on the prompt 1135, or a combination thereof.

In an illustrative example, the interface device(s) 1110 may receive the prompt 1135 as input(s) 1005 requesting information about a particular patient (e.g., a physiological attribute curve for the patient, or another one of the output(s) 1030). The device(s) 1110 extracts and/or generates the query 1130 with the name (or other identifier) of the particular patient, and initiates a query of the data store system(s) 1115 using the query 1130 to retrieve information 1140 for enhanced content 1145 indicating the patient's medical records, the patient's medical history, symptoms that the patient has reported, symptoms that a physician or nurse has identified or verified, information from medical textbooks and/or medical journals about those symptoms, diseases that the patient has been diagnosed with, information from medical textbooks and/or medical journals about those diseases, the patient's conversation history, and/or other context data about the patient. The data store system(s) 1115, the interface device(s) 1110, the LLM engine 1120, and/or another system can modify or enhance the prompt 1135 using this enhanced content 1145 (and/or information 1140) to generate an enhanced prompt 1150 that includes the enhanced content 1145 (e.g., the additional context data about the patient). In some examples, the data store system(s) 1115, the interface device(s) 1110, the LLM engine 1120, and/or another system can also fine-time the LLM(s) 1125 themselves based on the enhanced content 1145 and/or information 1140, for instance to prepare the LLM(s) 1125 to draw answers based on the enhanced content 1145 and/or information 1140 (e.g., based on the context data about the patient, based on the information from medical textbooks and/or medical journals, or a combination thereof). This enhanced prompt 1150 is processed using the LLM(s) 1125 (fine-tuned or not) to generate the response(s) 1155. In some examples, the LLM(s) 1125 can use the information 1140 and/or the enhanced content 1145 (that is in the enhanced prompt 1150, and/or that the LLM(s) 1125 are fine-tuned with) to help generate the response(s) 1155.

FIG. 12 is a conceptual diagram illustrating a process 1200 for dynamically updating an output 1240 (that is generated using ML model(s)) in a continuous fashion as further data continues to be received over time, in accordance with some examples.

A data stream 1205 is illustrated, and can represent, for instance, a stream of data to be input into the ML model(s) 1025 to generate an output 1240 (e.g., of one or more of the types of the output(s) 1030, such as the physiological attribute curve(s) 1035, physiological attribute value(s) 1040, and/or recommendation(s) 1042, and/or the RAG query(s) 1045). In some examples, the data stream 1205 is an example of at least a portion of the information 1010. In some examples, the data stream 1205 can include any of the types of data discussed with respect to the information 1010, such as user information, agent information, context information, or a combination thereof. The data stream 1205 includes large quantities of data that continue to be received over a long period of time. For instance, the data stream 1205 can include records of transactions that continue to occur over time. The output 1240 can be an example of one or more of the output(s) 1030, or vice versa.

In some examples, a system (e.g., the artificial intelligence system 1000, other systems discussed herein, or a combination thereof) can extract batches of data (e.g., batch 1210, batch 1220, batch 1230) from the data stream 1205 dynamically and in real-time (or near-real-time) as the data from the data stream 1205 continues to be received by the system. In some examples, the system can process the batches of data (e.g., using the ML model(s) 1025) dynamically and in real-time (or near-real-time) as the data from the data stream 1205 continues to be received by the system to generate update(s) to an output 1240 of the output(s) 1030. For instance, the batch 1210 undergoes processing 1212 to generate the update 1215. The batch 1220 undergoes processing 1222 to generate the update 1225. The batch 1230 undergoes processing 1232 to generate the update 1235. The system updates the output 1240 (e.g., using the ML model(s) 1025 of the artificial intelligence system 1000) based on the update 1215, the update 1225, and/or the update 1235, sequentially, in parallel, and/or in further batches of updates. For instance, the batch 1210, the batch 1220, the batch 1230, the update 1215, the update 1225, and/or the update 1235 can be added into the information 1010, the previous output(s) 1015, and/or can otherwise be added into the input(s) 1005. In this way, the system continues to update the output 1240 as the data from the data stream 1205 continues to be received by the system, so that the output 1240 is up-to-date with the changes to the data stream 1205.

In some examples, the data stream 1205 may include, for instance, at least a portion of the information 1010. In some examples, the processing 1212, the processing 1222, and/or the processing 1232, can include processing operations applied by the ML model(s) 1025 to the input(s) 1005 to generate the output(s) 1030. In some examples, the processing 1212, the processing 1222, and/or the processing 1232, can include processing operations such as normalization, reformatting, conversion between data types, rearrangement of data, removal of outliers, correction of errors, or a combination thereof. In some examples, the processing 1212, the processing 1222, and/or the processing 1232, can include processing operations using trained machine learning model(s) (e.g., the ML model(s) 1025) to generate updates to the output 1240. In some examples, the updates to the output 1240 (e.g., update 1215, update 1225, update 1235) can represent additional information (e.g., updates to the information 1010) to be input into the ML model(s) 1025 for analysis and updating of the output(s) 1030 (e.g., the output 1240). In some examples, the updates to the output 1240 are each new and updated instances of the output 1240. In some examples, the updates to the output 1240 include differences compared to a previous instance of the output 1240, so that the updates to the output 1240 can be combined with the previous instance of the output 1240 to generate an updated output 1240.

In some examples, the updates (e.g., update 1215, update 1225, update 1235) can include feedback 1055, training data, fine-tuning data, context data, model parameters (e.g., temperature, top P, frequency penalty, presence penalty and/or other parameters or settings) for training, re-training, fine-tuning, and/or updating the ML model(s) 1025 (e.g., as in the update 1060) in addition to, or instead of, updating the output 1240.

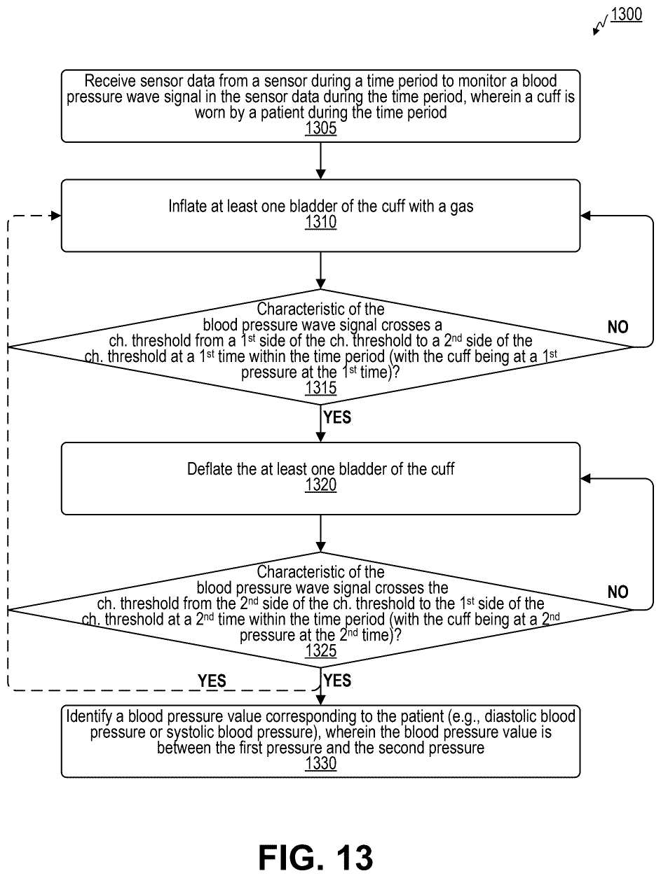

FIG. 13 is a flow diagram illustrating a process 1300 for physiological attribute analysis for a patient. The process 1300 can be performed by a physiological attribute monitoring system, which can include the physiological attribute monitoring system 100, the cuff 110, the pneumatic system 115, the monitoring system 120, a remote processing system or companion system that helps the monitoring system 120 with processing data, the physiological attribute monitoring system 200, the pump 250, the physiological attribute monitoring system 300, the compressed gas canister 350, the system performing the process 400, the physiological attribute monitoring system 500, the physiological attribute monitoring system 600, the physiological attribute monitoring system 800, the physiological attribute monitoring system 900, the artificial intelligence system 1000, the ML engine 1020, the ML model(s) 1025, the feedback engine(s) 1050, the interface device(s) 1110, the data store system(s) 1115, the LLM engine 1120, the LLM(s) 1125, the process 1200, the computing system 1400, a computing device, an apparatus, a processor executing instructions stored in a memory, a processor executing instructions stored in a non-transitory computer-readable storage medium, a component or sub-system of any of these systems, or a combination thereof.

At operation 1305, the physiological attribute monitoring system (or a subset or component thereof) is configured to, and can, receive sensor data from a sensor during a time period to monitor a blood pressure wave signal in the sensor data during the time period. A cuff 110 is worn by a patient 105 (e.g., on a body part 505) during the time period.

At operation 1310, the physiological attribute monitoring system (or a subset or component thereof) is configured to, and can, inflate (e.g., using the pneumatic system 115) at least one bladder (e.g., gas bladder(s) 124, gas bladders 515A-515E) of the cuff 110 with a gas (e.g., gas 815).

The term “inflate” as used in operation 1310 refers to increasing the amount of gas, and/or the gas pressure, in the at least one bladder of the cuff. In some examples, the inflation of operation 1310 can refer to inflating the at least one bladder of the cuff fully (e.g., until the at least one bladder of the cuff is full of the gas, or includes more than a threshold amount of the gas)-however, the inflation of operation 1310 should not be interpreted to require inflating the at least one bladder of the cuff fully. In some examples, the inflation of operation 1310 can refer to inflating the at least one bladder of the cuff by an increment (e.g., a specific amount of gas, or amount of gas pressure, transferred to the at least one bladder of the cuff) before the process 1300 proceeds from the operation 1310 to the operation 1315. In some examples, if the process 1300 returns to the operation 1310 after the operation 1315 (e.g., if the answer to the check in the operation 1315 is “no”), the inflation continues in an uninterrupted and/or continuous fashion.

At operation 1315, the physiological attribute monitoring system (or a subset or component thereof) is configured to, and can, determine if a characteristic (e.g., amplitude, slope) of the blood pressure wave signal crosses a threshold (e.g., threshold 270) from a first side of the characteristic threshold to a second side of the characteristic threshold (e.g., from below the characteristic threshold to above the characteristic threshold or vice versa) at a first time within the time period. The cuff is at a first pressure at the first time. If the characteristic of the blood pressure wave signal does not cross the characteristic threshold at the first time, the process 1300 returns to operation 1310 (to continue inflating the cuff) and/or restarts operation 1315. If the characteristic of the blood pressure wave signal does cross the characteristic threshold at the first time, the process 1300 continues from operation 1315 to operation 1320.

In some examples, operation 1310 and operation 1315, together, can refer to inflating at least one bladder of the cuff with a gas until the characteristic of the blood pressure wave signal crosses a characteristic threshold from a first side of the characteristic threshold to a second side of the characteristic threshold at a first time within the time period.

At operation 1320, the physiological attribute monitoring system (or a subset or component thereof) is configured to, and can, deflate the at least one bladder of the cuff.

The term “deflate” as used in operation 1320 refers to decreasing the amount of gas, and/or the gas pressure, in the at least one bladder of the cuff. In some examples, the deflation of operation 1320 can refer to deflating the at least one bladder of the cuff fully (e.g., until the at least one bladder of the cuff is empty of the gas, or includes less than a threshold amount of the gas)-however, the inflation of operation 1310 should not be interpreted to require deflating the at least one bladder of the cuff fully. In some examples, the deflation of operation 1320 can refer to deflating the at least one bladder of the cuff by an increment (e.g., a specific amount of gas, or amount of gas pressure, transferred out of the at least one bladder of the cuff) before the process 1300 proceeds from the operation 1320 to the operation 1325. In some examples, if the process 1300 returns to the operation 1320 after the operation 1325 (e.g., if the answer to the check in the operation 1325 is “no”), the deflation continues in an uninterrupted and/or continuous fashion.

At operation 1325, the physiological attribute monitoring system (or a subset or component thereof) is configured to, and can, determine if the characteristic (e.g., amplitude, slope) of the blood pressure wave signal crosses the characteristic threshold from the second side of the characteristic threshold to the first side of the characteristic threshold (e.g., from above the characteristic threshold to below the characteristic threshold or vice versa) at a second time within the time period. The cuff is at a second pressure at the second time. If the characteristic of the blood pressure wave signal does not cross the characteristic threshold at the second time, the process 1300 returns to operation 1320 (to continue deflating the cuff) and/or restarts operation 1325. If the characteristic of the blood pressure wave signal does cross the characteristic threshold at the second time, the process 1300 continues from operation 1325 to operation 1330 (and/or reverts back to operation 1310).

In some examples, operation 1320 and operation 1325, together, can refer to deflating the at least one bladder of the cuff until the characteristic of the blood pressure wave signal crosses the characteristic threshold from the second side of the characteristic threshold to the first side of the characteristic threshold at a second time within the time period.

At operation 1330, the physiological attribute monitoring system (or a subset or component thereof) is configured to, and can, identify a blood pressure value corresponding to the patient (e.g., diastolic blood pressure or systolic blood pressure), such that the blood pressure value is between the first pressure (of operation 1315) and the second pressure (of operation 1325).

In some aspects, the physiological attribute monitoring system (or a subset or component thereof) is configured to, and can, loop from operation 1325 back to operation 1310. The physiological attribute monitoring system can perform multiple cycles or loops of inflating the at least one bladder of the cuff and deflating the at least one bladder of the cuff, for instance to bring the upper bounds and lower bounds for the blood pressure value closer and closer to each other until they converge (e.g., until the difference 410 between the upper bounds and lower bounds for the blood pressure value are less than a difference threshold 420 apart from one another), as illustrated in the graph 320 (in FIG. 3) and/or the graph of the process 400 (in FIG. 4).

In some aspects, the blood pressure value is a diastolic blood pressure. In such aspects, the blood pressure wave signal is less than the characteristic threshold on the first side of the characteristic threshold, and the blood pressure wave signal is greater than the characteristic threshold on the second side of the characteristic threshold.

In some aspects, the blood pressure value is a systolic blood pressure. In such aspects, the blood pressure wave signal is greater than the characteristic threshold on the first side of the characteristic threshold, and the blood pressure wave signal is less than the characteristic threshold on the second side of the characteristic threshold.

In some aspects, the sensor is a microphone, the sensor data includes audio data recorded by the microphone, the blood pressure wave signal is a Korotkoff audio signal within the audio data, and the characteristic of the blood pressure wave signal is an amplitude of the Korotkoff audio signal.

In some aspects, the sensor is an electrical attribute sensor, wherein the sensor data includes electrical attribute data (e.g., voltage, resistance, current, impedance, or a combination thereof) measured by the electrical attribute sensor, and the blood pressure wave signal is an impedance signal based on the electrical attribute data.

In some aspects, inflating at least one bladder of the cuff with the gas (as in operation 1310) includes opening a valve (e.g., valve(s) 130, valve(s) 144, valves 525A-525E) to release an amount of the gas from a pressurized gas canister (e.g., compressed gas canister(s) 142, compressed gas canister 350) and to convey the amount of the gas from the pressurized gas canister to the at least one bladder of the cuff.

In some aspects, deflating the at least one bladder of the cuff (as in operation 1320) includes opening a valve (e.g., valve(s) 130, valve(s) 144, valves 525A-525E) to release an amount of the gas from the at least one bladder of the cuff.

In some aspects, the cuff includes a plurality of bladders (e.g., gas bladder(s) 124, gas bladders 515A-515E). Inflating the at least one bladder of the cuff with the gas includes inflating at least a subset of the plurality of bladders with the gas. Deflating the at least one bladder of the cuff includes deflating at least the subset of the plurality of bladders.

In some aspects, the at least one bladder includes a first bladder and a second bladder. In some aspects, inflating the at least one bladder of the cuff with the gas includes inflating the first bladder and the second bladder at a shared pressure (e.g., inflation rate, gas amount transferred into the at least one bladder), and deflating the at least one bladder of the cuff with the gas includes deflating the first bladder and the second bladder at a shared deflation rate. In some aspects, inflating the at least one bladder of the cuff with the gas includes inflating the first bladder at a first pressure (e.g., inflation rate, gas amount transferred into the first bladder) and inflating the second bladder at a second pressure (e.g., inflation rate, gas amount transferred into the second bladder). In some aspects, deflating the at least one bladder of the cuff with the gas includes deflating the first bladder at a first deflation rate (e.g., pressure, gas amount) and deflating the second bladder at a second deflation rate (e.g., pressure, gas amount).

In some aspects, the first bladder is closer to a heart of the patient than the second bladder, and wherein the first pressure (e.g., inflation rate, gas amount transferred into the at least one bladder) is lower than the second pressure (e.g., inflation rate, gas amount transferred into the at least one bladder). In some aspects, the cuff includes overlapping portions that overlap over each other while the cuff is worn by the patient during the time period. In some aspects, one of the overlapping portions includes the first bladder, and a non-overlapping portion of the cuff includes the second bladder. In such aspects, the first bladder and the second bladder can actually refer to gas pillows, such as the gas pillow(s) 126, the different gas pillows 530, and/or the different gas pillows 830.

In some aspects, the at least one bladder includes a first pillow and a second pillow. Inflating the at least one bladder of the cuff with the gas includes inflating the first pillow at a first pressure (e.g., inflation rate, gas amount transferred into the first pillow) and inflating the second pillow at a second pressure (e.g., inflation rate, gas amount transferred into the at least one bladder), and wherein deflating the at least one bladder of the cuff with the gas includes deflating the first pillow at a first deflation rate and deflating the second pillow at a second deflation rate, wherein the cuff includes overlapping portions that overlap over each other while the cuff is worn by the patient during the time period, wherein one of the overlapping portions includes the first pillow, and wherein a non-overlapping portion of the cuff includes the second pillow

In some aspects, the physiological attribute monitoring system (or a subset or component thereof) is configured to, and can, receive secondary sensor data from a second sensor during the time period to monitor a secondary blood pressure wave signal in the secondary sensor data during the time period. The physiological attribute monitoring system can inflate a second bladder of the cuff with the gas until the characteristic of the secondary blood pressure wave signal crosses a secondary threshold from a first side of the secondary threshold to a second side of the secondary threshold at a third time within the time period, wherein the second bladder is at a third pressure at the first time. physiological attribute monitoring system can deflate the second bladder of the cuff until the characteristic of the secondary blood pressure wave signal crosses the secondary threshold from the second side of the secondary threshold to the first side of the secondary threshold at a fourth time within the time period, wherein the second bladder is at a fourth pressure at the fourth time. The physiological attribute monitoring system can identify a second blood pressure value corresponding to the patient, wherein the second blood pressure value is between the third pressure and the fourth pressure. In some aspects, the first bladder and the second bladder are used to measure different types of blood pressure values. For instance, in some aspects, the blood pressure value is a diastolic blood pressure, and the second blood pressure value is a systolic blood pressure. In some aspects, the blood pressure value is a systolic blood pressure, and the second blood pressure value is a diastolic blood pressure.

In some aspects, the physiological attribute monitoring system (or a subset or component thereof) is configured to, and can, identify that a first fastener is in use to secure the cuff to a body part of the patient during the time period. The first fastener is one of a plurality of fasteners of the cuff. The physiological attribute monitoring system can identify a size of the body part of the patient based the first fastener being in use to secure the cuff to the body part of the patient during the time period. In some aspects, the physiological attribute monitoring system (or a subset or component thereof) is configured to, and can, set the characteristic threshold based the size of the body part of the patient.

In some aspects, the cuff includes at least one electromagnet. The first fastener includes a ferromagnetic material that is magnetically coupled to the at least one electromagnet of the cuff to secure the cuff to the body part of the patient during the time period. Identifying that the first fastener is in use to secure the cuff to the body part of the patient during the time period is based on detection of an electrical attribute corresponding to the electromagnet in the ferromagnetic material of the first fastener.

In some aspects, the plurality of fasteners include a plurality of electromagnets that each have different electrical attributes. The first fastener includes a first electromagnet of the plurality of electromagnets. Identifying that the first fastener is in use to secure the cuff to the body part of the patient during the time period is based on detection of an electrical attribute corresponding to the first electromagnet.

In some aspects, the sensor identifies that the at least one bladder is at the first pressure at the first time, and the sensor identifies that the at least one bladder is at the second pressure at the second time. In some aspects, a second sensor identifies that the at least one bladder is at the first pressure at the first time, and the second sensor identifies that the at least one bladder is at the second pressure at the second time.

In some aspects, the sensor (and/or the second sensor) captures serial measurements until it the serial measurements asymptotically converge. The serial measurements include at least at least one of the sensor data, the first pressure, or the second pressure.

In some aspects, identifying the blood pressure value corresponding to the patient (at operation 1330) is based on a binary search. In some examples, the binary search can stop early in case the system is able to measure the physiological attribute during the binary search. In some examples, the binary search can stop early in case the system is able to measure the physiological attribute during the binary search.

In some aspects, the physiological attribute monitoring system (or a subset or component thereof) is configured to, and can, identify a physiological attribute (other than the blood pressure value) based on at least one of the sensor data, the first pressure, or the second pressure. The physiological attribute includes at least one of a central venous pressure, a heart rate, a respiratory rate, or a cardiac output.

In some aspects, the physiological attribute monitoring system includes at least one valve and at least one actuator. In some aspects, to inflate the at least one bladder of the cuff (at operation 1310), the physiological attribute monitoring system (or a subset or component thereof) is configured to, and can, open the at least one valve using the at least one actuator to convey the gas into the at least one bladder from a gas source.

In some aspects, the physiological attribute monitoring system includes at least one valve and at least one actuator. In some aspects, to deflate the at least one bladder of the cuff (at operation 1320), the physiological attribute monitoring system (or a subset or component thereof) is configured to, and can, open the at least one valve using the at least one actuator to convey the gas out of the at least one bladder of the cuff.

The systems and methods disclosed herein solve a number of technical problems, and provide a number of technical improvements. For instance, the systems and methods described herein can improve the speed, efficiency, accuracy, flexibility, adaptability, and reliability of blood pressure monitoring.

In some examples, the physiological attribute monitoring system includes at least 1 cuff with at least one bladder, with at least zero divisions placed on at least one extremity or finger/toe. The various signals measured (with or without emitted or generated other signals) may be sampled at various frequencies (or continuously), by sensors (e.g. pressure) continuously (by transducers placed anywhere within the system) while bladder pressure being increased from 0 (or other preset low target number) to 200 mmHg (or other preset high target number) with a given rate (e.g. 0.1-20 mmHg/second) during inflation, and then decreased from 200 mmHg (or other preset high target number) to 0 (or other preset low target number) mmHg with a given rate (e.g. 0.1-20 mmHg/second) during deflation. The whole cycle of inflation and deflation can be repeated, or part of this cycle can be repeated, with same or variable (for each inflation-deflation cycle) low target number and high target number, until asymptotic convergency (asymptotic behavior) is detected by analysis of some or all signals. For example, a patient can walk into a physician's office. The physician can place the cuff 110 on the patient's arm. The physiological attribute monitoring system starts measuring, and the physiological attribute monitoring system starts inflating the cuff 110. Initially it may only inflate the cuff from 0 mmHg to 100 mmHg, (e.g., to find where the diastolic pressure is, and what are the best pressures in bladder(s)/cuff(s) to estimate it). But the physiological attribute monitoring system detects that between 0 mmHg and 80 mmHg, there was no signal variability at all (or very little), then the system detected that the same variability (estimated by AI algorithm(s) 158 and/or statistics) continued until 100 mmHg, so the physiological attribute monitoring system notices we need higher pressure in bladder to estimate diastolic pressure, and then physiological attribute monitoring system deflates down to 80 mmHg (not to 0 mmHg since there was no variability of pressure waveform below 80 mmHg) then inflate to 120 mmHg (once it detected that this was were the variability started changing, by AI, ML, or statistical means estimation) and then deflate to 80 mmHg, and inflate to 120 mmHg in cycle few times, until asymptotic convergency (asymptotic behavior) is detected in diastolic blood pressure estimation or heart rate. Once that is achieved, the physiological attribute monitoring system assumes the patient is rested enough and then the machine reports the final blood pressure value as the actual blood pressure value to the operator.

In some examples, after an asymptotic convergency is achieved (e.g., patient has rested enough for the patient's diastolic blood pressure to stabilize) the physiological attribute monitoring system, in some examples, then checks for systolic blood pressure once (assuming that is also subjective to adequate rest). In another example, to avoid mounting expensive sensors, the cuff 110 can include an inflatable structure, for instance including one or more of the gas bladder(s) 124 and/or gas pillow(s) 126, that inflates to about 40-50 mmHg, to be able to transduce pressures within the cuff 110. Pressures in this range may not be high enough to record any pulse wave variability with sufficient signal/noise ratio, but may be high enough to detect pressure changes caused by movement of the patient. This way, the physiological attribute monitoring system can detect movement using pressure sensors (e.g., pressure transducers), without any accelerometers, gyroscopes, and/or other sensors. Thus, the physiological attribute monitoring system can detect when patient rests for long enough before the physiological attribute monitoring system determines the patient's blood pressure.

In some aspects, the physiological attribute monitoring system (or a subset or component thereof) is configured to, and can, inflate a second bladder of the cuff with the gas (e.g., to 40-50 mmHg as discussed above). Before inflating the at least one bladder (at operation 1310), the physiological attribute monitoring system can determine, based on secondary sensor data from a second sensor (e.g., pressure sensor as discussed above, or accelerometer, gyrometer, IMU, and/or other sensor) that is coupled to the second bladder, that a movement level of the patient has remained below a movement level threshold for a predetermined amount of time (e.g., five minutes, N minutes).

In some examples, the physiological attribute monitoring system includes at least 1 cuff with at least one bladder, with at least zero divisions placed on at least one extremity or finger/toe. The various signals measured (with or without emitted or generated other signals) may be sampled at various frequencies (or continuously), by sensors (e.g. pressure) continuously (by transducers placed anywhere within the system) while bladder pressure being increased from 0 (or other preset low target number) to 200 mmHg (or other preset high target number) with a given rate (e.g. 0.1-20 mmHg/second) during inflation, and then decreased from 200 mmHg (or other preset high target number) to 0 (or other preset low target number) mmHg with a given rate (e.g. 0.1-20 mmHg/second) during deflation. The whole cycle of inflation and deflation can be repeated, with same (for each inflation-deflation cycle) low target number and high target number, until asymptotic convergency (asymptotic behavior) is detected by analysis of some or all signals, to estimate some, or all signals. For example patient walks into the office, and we place the bladder (cuff) on the patient's arm. We start measuring and system starts inflating the cuff from 0 mmHg to 200 mmHg and then deflate it back to 0 mmHg, in cycle few times, each time recording/measuring and analyzing some or signals, until asymptotic convergency (asymptotic behavior) is detected in diastolic blood pressure estimation, systolic blood pressure estimation or heart rate. Once that is achieved, system would assume patient rested enough and then the machine reports the final blood pressure as the actual to the operator.

In some examples, the physiological attribute monitoring system is able to determine optimal or current values for, and/or operator can set, parameters including but not limited to: time constant, damping ratio, natural frequency, poles and zeros, and/or system order. The physiological attribute monitoring system is able to determine optimal or current values for, and/or operator is able to set: signal-to-noise ratio, input frequency content, input magnitude, final value, initial conditions, decay/growth rate coefficient, settling time, convergence criteria, rice time, overshoot percentage. The physiological attribute monitoring system is able to determine optimal or current values for, and/or operator is able to set: temperature effects, load conditions, feedback mechanisms, nonlinearity, controller gains, compensator parameters, integrated/derivative faction, anti-windup limits.

All settings and measured and calculated parameters and signals, as discussed herein, can govern, initiate, or end the process of obtaining measurements of physiologic signals, when they become stable over the preset or established time, and they when they have low likelihood of changing over next time period (become stable enough to be considered at their asymptote).

In some examples, for instance, an operator can set the system to continue measuring the systolic blood pressure, until it detects its stability over the 3 minutes. The stability, for example, may be defined, as no more than 5% upward and downward changes of systolic blood pressure from the moving average of systolic blood pressure measured/estimated/calculated over the last 2 minutes.

Other means of defining asymptotic convergence or the end of measurement/calculation/estimation may be defined using parameters and/or measures or statistics such as: time constant, damping ratio, natural frequency, poles and zeros, system order. The physiological attribute monitoring system is able to determine optimal or current values for, and/or operator is able to set: signal-to-noise ratio, input frequency content, input magnitude, final value, initial conditions, decay/growth rate coefficient, settling time, convergence criteria, rice time, overshoot percentage. The physiological attribute monitoring system is able to determine optimal or current values for, and/or operator is able to set: temperature effects, load conditions, feedback mechanisms, nonlinearities, controller gains, compensator parameters, integrated/derivative faction, anti-wind up limits.

In some examples, the physiological attribute monitoring system can include a built in heater or cooler in the bladder or within the gas pumps, or gas delivery systems, to use gas with specific temperature (e.g., body temperature), for instance to avoid vasoconstriction or other effects, or effects due to low or high temperature that cause an impact on measured signals or accuracy of measurements. The bladders may include heating and/or cooling systems. Add this as claim

In some examples, the physiological attribute monitoring system can additionally or alternatively monitor the flow in the vessels of the body part 505 of the patient 105 with ultrasound and/or doppler, the cuff would have few (e.g. 4) scattered Doppler ultrasound probes along the circumference of the cuff 110, angled at a low possible angle below a threshold relative to the surface of the cuff 110 (e.g., as parallel to the surface of the cuff 110 as possible, for instance less than 45 degrees to the skin surface, for the most sensitive measurements). These probes record relative flow rate in body parts' arteries, as the cuff is being inflated or deflated. Any lowest pressure registered when there is continuous flow in arteries at first in the arterial direction (from heart to tip of body part) would be diastolic blood pressure. Any highest pressure, registered, when there is any maximal velocity flow from all velocities recorded, would be systolic blood pressure. Any lowest pressure registered pressure when there is continuous flow in vessels of the body part at first noticed while the pressure in cuff is decreasing in the venous direction (from tip of body part to the heart) would be venous pressure. This could be average between the few beats or few runs to register mean venous pressure, or central venous pressure (CVP). CVP is a good clinical indicator volume status, and many times guides therapy in heart failure, cardiac ICU, and so forth. CVP can guide therapy to avoid hospital readmission, orthostatic symptoms during hypertension therapy).

In some examples, the physiological attribute monitoring system 500 physiological attribute monitoring system can use the techniques discussed herein to measure blood pressure momentarily. Thus, all measurements can be repeated a few times within seconds, for instance to register average values for even better accuracy, especially in case patient moves, or there is arrhythmia, or other noise.

In some examples, the physiological attribute monitoring system can additionally or alternatively monitor the flow in the body part's vessels with electromagnetic field(s). For instance, in such scenarios, electromagnetic induction is used to measure arterial and venous flow rate changes. Any lowest pressure registered when there is continuous flow in arteries at first in the arterial direction (from heart to tip of body part) would be diastolic blood pressure. Any highest pressure, registered, when there is any maximal velocity flow from all velocities recorded, would be systolic blood pressure. Any lowest pressure registered pressure when there is continuous flow in the vessels of the body part at first noticed while the pressure in cuff is decreasing in the venous direction (from tip of body part to the heart) would be venous pressure (this could be average between the few beets or few runs to register mean venous pressure, or central venous pressure (CVP).

In some examples, when blood pressure measurements are repeatedly performed, until given variability between the reads drops to E % over the given last N seconds (when all or given percent subsequent last blood pressure reads recorded within last N seconds for all or any systolic, diastolic of CVP fall between moving average+−E %).

In some examples, to differentiate between arterial and venous flow, the physiological attribute monitoring system use digital subtraction. In some examples, the physiological attribute monitoring system can digitally subtract the arterial flow signals from all signals, using the observation that arterial signals prevail and are recorded without any venous signals until the pressures in the cuff drop to much lower than diastolic. For example, a typical healthy patient has the following blood pressure values: Systolic 120 mmHg, Diastolic 80 mmHg, and central venous pressure <15 mmHg. All the signals recorded by the probes (either electromagnetic or doppler) until the cuff pressure drops to 20 are from arteries, as there is no flow in veins yet. Only after pressure in cuff drops to below 15, we will start seeing signals from arteries and veins together (all signals). The physiological attribute monitoring system can subtract signals from arteries from all signals, leaving the venous signals as a result. In some examples, the digital subtraction can be done using AI algorithm(s) 158, such as the ML model(s) 1025. In some examples, training of the AI algorithm(s) 158 (e.g., the ML model(s) 1025) to perform this digital subtraction can be done using training data from a database of all signals, with signals annotated by venous, arterial, and “all”. Another way to train the AI algorithm(s) 158 (e.g., the ML model(s) 1025) to perform this digital subtraction is to perform AI signals analysis with recorded systolic, diastolic and CVP pressure in patients who have arterial lines in and central lines in ICUs/cardiac catheterization labs and maybe in few healthy/hypertensive individuals, and who have all these pressures recorded, sometimes even real time (e.g., in ICU, cardiac surgeries, cardiac catheterization labs).

In some examples, an artificial limb, such as an artificial arm, could be used to test, train, validate, calibrate, and/or compare physiological attribute monitoring systems. Such an artificial body part can have a fluid pumped through it at regular intervals, simulating blood flow. Such an artificial body part can be referred to as a phantom body part, or just a phamtom, herein.

Such a phantom can be used to artificially produce preset systolic and preset diastolic pressure, simulate arteries, that will be used to validate blood pressure monitors and their accuracy to given pressures produced by pump of phantom. The phantom can include of customizable pump (e.g., producing blood pressure as heart) with compliant balloon “aortic” buffer (e.g., serving as an aorta) and valve (e.g., serving as an aortic valve), and another “ventricular” buffer (e.g., serving as a left ventricle).

The pump generates continuous pressure to fill the ventricular buffer with the preset “systolic” pressure. The “aortic” valve (between the “ventricular” buffer and “aortic” buffers) opens to imitate systole, and releases the fluid, imitating “blood,” from “ventricular” buffer into the “aortic” buffer. The fluid used can be selected to have viscosity, density, temperature, and other parameters similar to real blood. “Aortic” buffer intakes the bolus of fluid, due its compliance, dilating, and then slowly (after the “aortic valve” closes) releases the fluid into the arteries.

In some examples, the phantom can have artificial arm(s) (upper arms) and/or wrists (left and right) with appropriate anatomy (e.g., human left upper extremity and right extremity, especially vascular, anatomy, with appropriate veins and arteries).

In some examples, in the phantom, the veins of the arm return the “blood” into the “ventricular” buffer via larger veins and pump, or through the “blood” circuit outside of patient. The aortic buffer comes in various compliance and sizes, which renders various “pulse amplitude”-difference between systolic and diastolic pressure, please note that the pump will be preset to various “systolic pressures”, but the diastolic pressure will be result of the variable compliance and size of “aortic” buffer.

This phantom can help the physiological attribute monitoring system to correlate: systolic, diastolic, and mean pressures measured on arms (left and right); systolic, diastolic, and mean pressures measured on forearms (left and right); systolic, diastolic, and mean pressures measured in aorta (“aortic” buffer); systolic, diastolic, and mean pressures measured in ventricle (“ventricular” buffer); and systolic, diastolic, and mean pressures measured in radial artery by “a-line.” These pressures can all be measured with sensors (e.g., pressure transducers) placed in various places at the physiological attribute monitoring system and/or the phantom. In some examples, the sizes of arms and/or forearms of the phantom can be customizable for this phantom, so cuffs with all sizes could be validated and calibrated. This phantom could be used for cardiac output correlations with real time pulse wave analysis, and for validation of impedance based cardiac output measuring devices. The phantom can also include sensors (e.g., pressure transducers) in the “aortic” buffer, in the major arteries (in the “upper arm”, and in “lower arm” (forearm), and in “radial artery” top imitate A-line pressure measurements. Use of a phantom can allow for training, validation, and/or calibration that is precise (repetitive), automated, and faster (e.g., can be done robotically). The phantom can be referred to as a phantom body part, a simulated body part, a phantom limb, a simulated limb, a phantom extremity, a simulated extremity, or a combination thereof.

In some aspects, the physiological attribute monitoring system (or a subset or component thereof) is configured to, and can, calibrate the sensor (and/or other portions of the physiological attribute monitoring system) using a simulated body part (e.g., phantom body part). The simulated body part includes a liquid pumped through a channel at a predetermined pressure. In some examples, calibrating the sensor includes matching a pressure measurement from the sensor to the predetermined pressure.

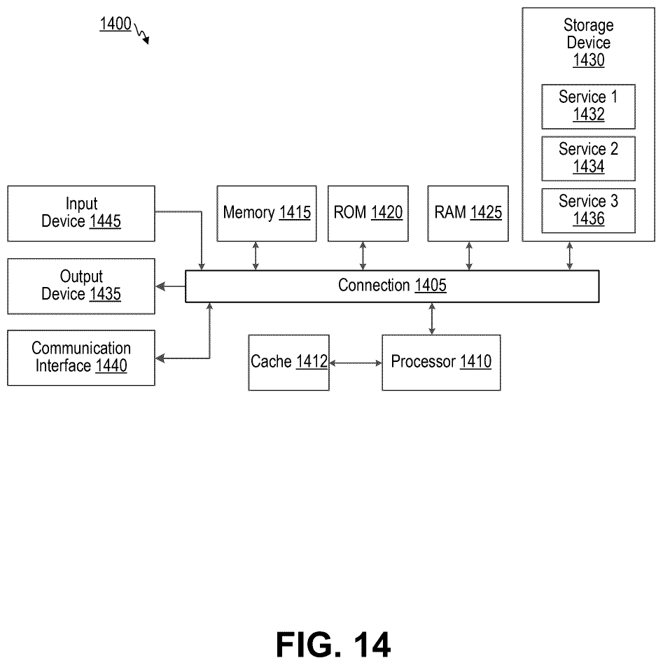

FIG. 14 is a diagram illustrating an example of a system for implementing certain aspects of the present technology. In particular, FIG. 14 illustrates an example of computing system 1400, which can be for example any computing device making up internal computing system, a remote computing system, a camera, or any component thereof in which the components of the system are in communication with each other using connection 1405. Connection 1405 can be a physical connection using a bus, or a direct connection into processor 1410, such as in a chipset architecture. Connection 1405 can also be a virtual connection, networked connection, or logical connection.

In some embodiments, computing system 1400 is a distributed system in which the functions described in this disclosure can be distributed within a datacenter, multiple data centers, a peer network, etc. In some embodiments, one or more of the described system components represents many such components each performing some or all of the function for which the component is described. In some embodiments, the components can be physical or virtual devices.

Example system 1400 includes at least one processing unit (CPU or processor) 1410 and connection 1405 that couples various system components including system memory 1415, such as read-only memory (ROM) 1420 and random access memory (RAM) 1425 to processor 1410. Computing system 1400 can include a cache 1412 of high-speed memory connected directly with, in close proximity to, or integrated as part of processor 1410.

Processor 1410 can include any general purpose processor and a hardware service or software service, such as services 1432, 1434, and 1436 stored in storage device 1430, configured to control processor 1410 as well as a special-purpose processor where software instructions are incorporated into the actual processor design. Processor 1410 may essentially be a completely self-contained computing system, containing multiple cores or processors, a bus, memory controller, cache, etc. A multi-core processor may be symmetric or asymmetric.

To enable user interaction, computing system 1400 includes an input device 1445, which can represent any number of input mechanisms, such as a microphone for speech, a touch-sensitive screen for gesture or graphical input, keyboard, mouse, motion input, speech, etc. Computing system 1400 can also include output device 1435, which can be one or more of a number of output mechanisms. In some instances, multimodal systems can enable a user to provide multiple types of input/output to communicate with computing system 1400. Computing system 1400 can include communication interface 1440, which can generally govern and manage the user input and system output. The communication interface may perform or facilitate receipt and/or transmission wired or wireless communications using wired and/or wireless transceivers, including those making use of an audio jack/plug, a microphone jack/plug, a universal serial bus (USB) port/plug, an Apple® Lightning® port/plug, an Ethernet port/plug, a fiber optic port/plug, a proprietary wired port/plug, a BLUETOOTH® wireless signal transfer, a BLUETOOTH® low energy (BLE) wireless signal transfer, an IBEACON® wireless signal transfer, a radio-frequency identification (RFID) wireless signal transfer, near-field communications (NFC) wireless signal transfer, dedicated short range communication (DSRC) wireless signal transfer, 802.11 Wi-Fi wireless signal transfer, wireless local area network (WLAN) signal transfer, Visible Light Communication (VLC), Worldwide Interoperability for Microwave Access (WiMAX), Infrared (IR) communication wireless signal transfer, Public Switched Telephone Network (PSTN) signal transfer, Integrated Services Digital Network (ISDN) signal transfer, 3G/4G/5G/LTE cellular data network wireless signal transfer, ad-hoc network signal transfer, radio wave signal transfer, microwave signal transfer, infrared signal transfer, visible light signal transfer, ultraviolet light signal transfer, wireless signal transfer along the electromagnetic spectrum, or some combination thereof. The communication interface 1440 may also include one or more Global Navigation Satellite System (GNSS) receivers or transceivers that are used to determine a location of the computing system 1400 based on receipt of one or more signals from one or more satellites associated with one or more GNSS systems. GNSS systems include, but are not limited to, the US-based Global Positioning System (GPS), the Russia-based Global Navigation Satellite System (GLONASS), the China-based BeiDou Navigation Satellite System (BDS), and the Europe-based Galileo GNSS. There is no restriction on operating on any particular hardware arrangement, and therefore the basic features here may easily be substituted for improved hardware or firmware arrangements as they are developed.

Storage device 1430 can be a non-volatile and/or non-transitory and/or computer-readable memory device and can be a hard disk or other types of computer readable media which can store data that are accessible by a computer, such as magnetic cassettes, flash memory cards, solid state memory devices, digital versatile disks, cartridges, a floppy disk, a flexible disk, a hard disk, magnetic tape, a magnetic strip/stripe, any other magnetic storage medium, flash memory, memristor memory, any other solid-state memory, a compact disc read only memory (CD-ROM) optical disc, a rewritable compact disc (CD) optical disc, digital video disk (DVD) optical disc, a blu-ray disc (BDD) optical disc, a holographic optical disk, another optical medium, a secure digital (SD) card, a micro secure digital (microSD) card, a Memory Stick® card, a smartcard chip, a EMV chip, a subscriber identity subsystem (SIM) card, a mini/micro/nano/pico SIM card, another integrated circuit (IC) chip/card, random access memory (RAM), static RAM (SRAM), dynamic RAM (DRAM), read-only memory (ROM), programmable read-only memory (PROM), erasable programmable read-only memory (EPROM), electrically erasable programmable read-only memory (EEPROM), flash EPROM (FLASHEPROM), cache memory (L1/L2/L3/L4/L5/etc.), resistive random-access memory (RRAM/ReRAM), phase change memory (PCM), spin transfer torque RAM (STT-RAM), another memory chip or cartridge, and/or a combination thereof.

The storage device 1430 can include software services, servers, services, etc., that when the code that defines such software is executed by the processor 1410, it causes the system to perform a function. In some embodiments, a hardware service that performs a particular function can include the software component stored in a computer-readable medium in connection with the necessary hardware components, such as processor 1410, connection 1405, output device 1435, etc., to carry out the function.

As used herein, the term “computer-readable medium” includes, but is not limited to, portable or non-portable storage devices, optical storage devices, and various other mediums capable of storing, containing, or carrying instruction(s) and/or data. A computer-readable medium may include a non-transitory medium in which data can be stored and that does not include carrier waves and/or transitory electronic signals propagating wirelessly or over wired connections. Examples of a non-transitory medium may include, but are not limited to, a magnetic disk or tape, optical storage media such as compact disk (CD) or digital versatile disk (DVD), flash memory, memory or memory devices. A computer-readable medium may have stored thereon code and/or machine-executable instructions that may represent a procedure, a function, a subprogram, a program, a routine, a subroutine, a subsystem, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted using any suitable means including memory sharing, message passing, token passing, network transmission, or the like.

In some embodiments the computer-readable storage devices, mediums, and memories can include a cable or wireless signal containing a bit stream and the like. However, when mentioned, non-transitory computer-readable storage media expressly exclude media such as energy, carrier signals, electromagnetic waves, and signals per se.

Specific details are provided in the description above to provide a thorough understanding of the embodiments and examples provided herein. However, it will be understood by one of ordinary skill in the art that the embodiments may be practiced without these specific details. For clarity of explanation, in some instances the present technology may be presented as including individual functional blocks including functional blocks comprising devices, device components, steps or routines in a method embodied in software, or combinations of hardware and software. Additional components may be used other than those shown in the figures and/or described herein. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

Individual embodiments may be described above as a process or method which is depicted as a flowchart, a flow diagram, a data flow diagram, a structure diagram, or a block diagram. Although a flowchart may describe the operations as a sequential process, many of the operations can be performed in parallel or concurrently. In addition, the order of the operations may be re-arranged. A process is terminated when its operations are completed, but could have additional steps not included in a figure. A process may correspond to a method, a function, a procedure, a subroutine, a subprogram, etc. When a process corresponds to a function, its termination can correspond to a return of the function to the calling function or the main function.

Processes and methods according to the above-described examples can be implemented using computer-executable instructions that are stored or otherwise available from computer-readable media. Such instructions can include, for example, instructions and data which cause or otherwise configure a general purpose computer, special purpose computer, or a processing device to perform a certain function or group of functions. Portions of computer resources used can be accessible over a network. The computer executable instructions may be, for example, binaries, intermediate format instructions such as assembly language, firmware, source code, etc. Examples of computer-readable media that may be used to store instructions, information used, and/or information created during methods according to described examples include magnetic or optical disks, flash memory, USB devices provided with non-volatile memory, networked storage devices, and so on.

Devices implementing processes and methods according to these disclosures can include hardware, software, firmware, middleware, microcode, hardware description languages, or any combination thereof, and can take any of a variety of form factors. When implemented in software, firmware, middleware, or microcode, the program code or code segments to perform the necessary tasks (e.g., a computer-program product) may be stored in a computer-readable or machine-readable medium. A processor(s) may perform the necessary tasks. Typical examples of form factors include laptops, smart phones, mobile phones, tablet devices or other small form factor personal computers, personal digital assistants, rackmount devices, standalone devices, and so on. Functionality described herein also can be embodied in peripherals or add-in cards. Such functionality can also be implemented on a circuit board among different chips or different processes executing in a single device, by way of further example.

The instructions, media for conveying such instructions, computing resources for executing them, and other structures for supporting such computing resources are example means for providing the functions described in the disclosure.

In the foregoing description, aspects of the application are described with reference to specific embodiments thereof, but those skilled in the art will recognize that the application is not limited thereto. Thus, while illustrative embodiments of the application have been described in detail herein, it is to be understood that the inventive concepts may be otherwise variously embodied and employed, and that the appended claims are intended to be construed to include such variations, except as limited by the prior art. Various features and aspects of the above-described application may be used individually or jointly. Further, embodiments can be utilized in any number of environments and applications beyond those described herein without departing from the broader spirit and scope of the specification. The specification and drawings are, accordingly, to be regarded as illustrative rather than restrictive. For the purposes of illustration, methods were described in a particular order. It should be appreciated that in alternate embodiments, the methods may be performed in a different order than that described.

One of ordinary skill will appreciate that the less than (“<”) and greater than (“>”) symbols or terminology used herein can be replaced with less than or equal to (“≤”) and greater than or equal to (“≥”) symbols, respectively, without departing from the scope of this description.

Where components are described as being “configured to” perform certain operations, such configuration can be accomplished, for example, by designing electronic circuits or other hardware to perform the operation, by programming programmable electronic circuits (e.g., microprocessors, or other suitable electronic circuits) to perform the operation, or any combination thereof.

The phrase “coupled to” refers to any component that is physically connected to another component either directly or indirectly, and/or any component that is in communication with another component (e.g., connected to the other component over a wired or wireless connection, and/or other suitable communication interface) either directly or indirectly.

Claim language or other language reciting “at least one of” a set and/or “one or more” of a set indicates that one member of the set or multiple members of the set (in any combination) satisfy the claim. For example, claim language reciting “at least one of A and B” means A, B, or A and B. In another example, claim language reciting “at least one of A, B, and C” means A, B, C, or A and B, or A and C, or B and C, or A and B and C. The language “at least one of” a set and/or “one or more” of a set does not limit the set to the items listed in the set. For example, claim language reciting “at least one of A and B” can mean A, B, or A and B, and can additionally include items not listed in the set of A and B.

The various illustrative logical blocks, subsystems, circuits, and algorithm steps described in connection with the embodiments disclosed herein may be implemented as electronic hardware, computer software, firmware, or combinations thereof. To clearly illustrate this interchangeability of hardware and software, various illustrative components, blocks, subsystems, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present application.

The techniques described herein may also be implemented in electronic hardware, computer software, firmware, or any combination thereof. Such techniques may be implemented in any of a variety of devices such as general purposes computers, wireless communication device handsets, or integrated circuit devices having multiple uses including application in wireless communication device handsets and other devices. Any features described as subsystems or components may be implemented together in an integrated logic device or separately as discrete but interoperable logic devices. If implemented in software, the techniques may be realized at least in part by a computer-readable data storage medium comprising program code including instructions that, when executed, performs one or more of the methods described above. The computer-readable data storage medium may form part of a computer program product, which may include packaging materials. The computer-readable medium may comprise memory or data storage media, such as random access memory (RAM) such as synchronous dynamic random access memory (SDRAM), read-only memory (ROM), non-volatile random access memory (NVRAM), electrically erasable programmable read-only memory (EEPROM), FLASH memory, magnetic or optical data storage media, and the like. The techniques additionally, or alternatively, may be realized at least in part by a computer-readable communication medium that carries or communicates program code in the form of instructions or data structures and that can be accessed, read, and/or executed by a computer, such as propagated signals or waves.

The program code may be executed by a processor, which may include one or more processors, such as one or more digital signal processors (DSPs), general purpose microprocessors, an application specific integrated circuits (ASICs), field programmable logic arrays (FPGAs), or other equivalent integrated or discrete logic circuitry. Such a processor may be configured to perform any of the techniques described in this disclosure. A general purpose processor may be a microprocessor; but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration. Accordingly, the term “processor,” as used herein may refer to any of the foregoing structure, any combination of the foregoing structure, or any other structure or apparatus suitable for implementation of the techniques described herein. In addition, in some aspects, the functionality described herein may be provided within dedicated software subsystems or hardware subsystems configured for encoding and decoding, or incorporated in a combined video encoder-decoder (CODEC).

While various flow diagrams and block diagrams provided and described above may show a particular order of operations performed by some embodiments of the subject technology, it should be understood that such order is exemplary. Alternative embodiments may perform the operations in a different order, combine certain operations, overlap certain operations, or some combination thereof. It should be understood that unless disclosed otherwise, any process illustrated in any flow diagram herein or otherwise illustrated or described herein may be performed by a machine, mechanism, and/or computing system 1400 discussed herein, and may be performed automatically (e.g., in response to one or more triggers/conditions described herein), autonomously, semi-autonomously (e.g., based on received instructions), or a combination thereof. Furthermore, any action described herein as occurring in response to one or more particular triggers/conditions should be understood to optionally occur automatically in response to the one or more particular triggers/conditions.

The foregoing detailed description of the technology has been presented for purposes of illustration and description. It is not intended to be exhaustive or to limit the technology to the precise form disclosed. Many modifications and variations are possible in light of the above teaching. The described embodiments were chosen in order to best explain the principles of the technology, its practical application, and to enable others skilled in the art to utilize the technology in various embodiments and with various modifications as are suited to the particular use contemplated. It is intended that the scope of the technology be defined by the claim.

Illustrative aspects of the disclosure include:
Aspect 1. A method of optimizing a blood pressure analysis for a patient, the method comprising: receiving sensor data from a sensor during a time period to monitor a characteristic of a blood pressure wave signal in the sensor data during the time period, wherein a cuff is worn by the patient during the time period; inflating at least one bladder of the cuff with a gas until the characteristic of the blood pressure wave signal crosses a characteristic threshold from a first side of the characteristic threshold to a second side of the characteristic threshold at a first time within the time period, wherein the at least one bladder is at a first pressure at the first time; deflating the at least one bladder of the cuff until the characteristic of the blood pressure wave signal crosses the characteristic threshold from the second side of the characteristic threshold to the first side of the characteristic threshold at a second time within the time period, wherein the at least one bladder is at a second pressure at the second time; and identifying a blood pressure value corresponding to the patient, wherein the blood pressure value is between the first pressure and the second pressure.
Aspect 2. The method of Aspect 1, wherein the blood pressure value is a diastolic blood pressure, wherein the blood pressure wave signal is less than the characteristic threshold on the first side of the characteristic threshold, and wherein the blood pressure wave signal is greater than the characteristic threshold on the second side of the characteristic threshold.
Aspect 3. The method of any one of Aspects 1 to 2, wherein the blood pressure value is a systolic blood pressure, wherein the blood pressure wave signal is greater than the characteristic threshold on the first side of the characteristic threshold, and wherein the blood pressure wave signal is less than the characteristic threshold on the second side of the characteristic threshold.
Aspect 4. The method of any one of Aspects 1 to 3, wherein the sensor is a microphone, wherein the sensor data includes audio data recorded by the microphone, wherein the blood pressure wave signal is a Korotkoff audio signal within the audio data, and wherein the characteristic of the blood pressure wave signal is an amplitude of the Korotkoff audio signal.
Aspect 5. The method of any one of Aspects 1 to 4, wherein the sensor is an electrical attribute sensor, wherein the sensor data includes electrical attribute data measured by the electrical attribute sensor, and wherein the blood pressure wave signal is an impedance signal based on the electrical attribute data.
Aspect 6. The method of any one of Aspects 1 to 5, wherein inflating at least one bladder of the cuff with the gas includes opening a valve to release an amount of the gas from a pressurized gas canister and to convey the amount of the gas from the pressurized gas canister to the at least one bladder of the cuff.
Aspect 7. The method of any one of Aspects 1 to 6, wherein deflating the at least one bladder of the cuff includes opening a valve to release an amount of the gas from the at least one bladder of the cuff.
Aspect 8. The method of any one of Aspects 1 to 7, wherein the cuff includes a plurality of bladders, wherein inflating the at least one bladder of the cuff with the gas includes inflating at least a subset of the plurality of bladders with the gas, and wherein deflating the at least one bladder of the cuff includes deflating at least the subset of the plurality of bladders.
Aspect 9. The method of any one of Aspects 1 to 8, wherein the at least one bladder includes a first bladder and a second bladder, wherein inflating the at least one bladder of the cuff with the gas includes inflating the first bladder and the second bladder at a shared pressure.
Aspect 10. The method of any one of Aspects 1 to 9, wherein the at least one bladder includes a first bladder and a second bladder, wherein inflating the at least one bladder of the cuff with the gas includes inflating the first bladder at a first pressure and inflating the second bladder at a second pressure.
Aspect 11. The method of Aspect 10, wherein the first bladder is closer to a heart of the patient than the second bladder, and wherein the first pressure is lower than the second pressure.
Aspect 12. The method of any one of Aspects 1 to 11, wherein the at least one bladder includes a first pillow and a second pillow, wherein inflating the at least one bladder of the cuff with the gas includes inflating the first pillow at a first inflation rate and inflating the second pillow at a second inflation rate, and wherein deflating the at least one bladder of the cuff with the gas includes deflating the first pillow at a first deflation rate and deflating the second pillow at a second deflation rate, wherein the cuff includes overlapping portions that overlap over each other while the cuff is worn by the patient during the time period, wherein one of the overlapping portions includes the first pillow, and wherein a non-overlapping portion of the cuff includes the second pillow.
Aspect 13. The method of any one of Aspects 1 to 12, further comprising: receive secondary sensor data from a second sensor during the time period to monitor a secondary blood pressure wave signal in the secondary sensor data during the time period; inflate a second bladder of the cuff with the gas until the characteristic of the secondary blood pressure wave signal crosses a secondary threshold from a first side of the secondary threshold to a second side of the secondary threshold at a third time within the time period, wherein the second bladder is at a third pressure at the first time; deflate the second bladder of the cuff until the characteristic of the secondary blood pressure wave signal crosses the secondary threshold from the second side of the secondary threshold to the first side of the secondary threshold at a fourth time within the time period, wherein the second bladder is at a fourth pressure at the fourth time; and identify a second blood pressure value corresponding to the patient, wherein the second blood pressure value is between the third pressure and the fourth pressure.
Aspect 14. The method of Aspect 13, wherein the blood pressure value is a diastolic blood pressure, and wherein the second blood pressure value is a systolic blood pressure.
Aspect 15. The method of any one of Aspects 13 to 14, wherein the blood pressure value is a systolic blood pressure, and wherein the second blood pressure value is a diastolic blood pressure.
Aspect 16. The method of any one of Aspects 1 to 15, further comprising: identifying that a first fastener is in use to secure the cuff to a body part of the patient during the time period, wherein the first fastener is one of a plurality of fasteners of the cuff; and identifying a size of the body part of the patient based the first fastener being in use to secure the cuff to the body part of the patient during the time period.
Aspect 17. The method of Aspect 16, further comprising: setting the characteristic threshold based the size of the body part of the patient.
Aspect 18. The method of any one of Aspects 16 to 17, wherein the cuff includes at least one electromagnet, wherein the first fastener includes a ferromagnetic material that is magnetically coupled to the at least one electromagnet of the cuff to secure the cuff to the body part of the patient during the time period, and wherein identifying that the first fastener is in use to secure the cuff to the body part of the patient during the time period is based on detection of an electrical attribute corresponding to the electromagnet in the ferromagnetic material of the first fastener.
Aspect 19. The method of any one of Aspects 16 to 18, wherein plurality of fasteners include a plurality of electromagnets that each have different electrical attributes, wherein the first fastener includes a first electromagnet of the plurality of electromagnets, and wherein identifying that the first fastener is in use to secure the cuff to the body part of the patient during the time period is based on detection of an electrical attribute corresponding to the first electromagnet.
Aspect 20. The method of any one of Aspects 1 to 19, further comprising: inflating a second bladder of the cuff with the gas; and determining, based on secondary sensor data from a second sensor that is coupled to the second bladder, and before inflating the at least one bladder, that a movement level of the patient has remained below a movement level threshold for a predetermined amount of time.
Aspect 21. The method of any one of Aspects 1 to 20, further comprising: calibrating the sensor using a simulated body part, wherein the simulated body part includes a liquid pumped through a channel at a predetermined pressure, and wherein calibrating the sensor includes matching a pressure measurement from the sensor to the predetermined pressure.
Aspect 22. The method of any one of Aspects 1 to 21, wherein the sensor identifies that the at least one bladder is at the first pressure at the first time, and wherein the sensor identifies that the at least one bladder is at the second pressure at the second time.
Aspect 23. The method of any one of Aspects 1 to 22, wherein a second sensor identifies that the at least one bladder is at the first pressure at the first time, and wherein the second sensor identifies that the at least one bladder is at the second pressure at the second time.
Aspect 24. The method of any one of Aspects 1 to 23, wherein identifying the blood pressure value corresponding to the patient is based on a binary search.
Aspect 25. The method of any one of Aspects 1 to 24, wherein the sensor captures serial measurements until it the serial measurements asymptotically converge, wherein the serial measurements include at least at least one of the sensor data, the first pressure, or the second pressure.
Aspect 26. The method of any one of Aspects 1 to 25, further comprising: identifying a physiological attribute based on at least one of the sensor data, the first pressure, or the second pressure, wherein the physiological attribute includes at least one of a central venous pressure, a heart rate, a respiratory rate, or a cardiac output.
Aspect 27. A system for blood pressure analysis for a patient, the system comprising: a memory storing instructions; and a processor that executes the instructions, wherein execution of the instructions by the processor causes the processor to: receive sensor data from a sensor during a time period to monitor a characteristic of a blood pressure wave signal in the sensor data during the time period, wherein a cuff is worn by the patient during the time period; inflate at least one bladder of the cuff with a gas until the characteristic of the blood pressure wave signal crosses a characteristic threshold from a first side of the characteristic threshold to a second side of the characteristic threshold at a first time within the time period, wherein the at least one bladder is at a first pressure at the first time; deflate the at least one bladder of the cuff until the characteristic of the blood pressure wave signal crosses the characteristic threshold from the second side of the characteristic threshold to the first side of the characteristic threshold at a second time within the time period, wherein the at least one bladder is at a second pressure at the second time; and identify a blood pressure value corresponding to the patient, wherein the blood pressure value is between the first pressure and the second pressure.
Aspect 28. The system of Aspect 27, further comprising: at least one valve; and at least one actuator, wherein, to inflate the at least one bladder of the cuff, the execution of the instructions by the processor causes the processor to open the at least one valve using the at least one actuator to convey the gas into the at least one bladder from a gas source.
Aspect 29. The system of any one of Aspects 27 or 28, further comprising: at least one valve; and at least one actuator, wherein, to deflate the at least one bladder of the cuff, the execution of the instructions by the processor causes the processor to open the at least one valve using the at least one actuator to convey the gas out of the at least one bladder of the cuff.
Aspect 30. The system of any one of Aspects 27 to 29, wherein the execution of the instructions by the processor causes the processor to perform operations according to any one of Aspects 2 to 26.
Aspect 31. A non-transitory computer readable storage medium having embodied thereon a program, wherein the program is executable by a processor to perform a method of blood pressure analysis for a patient, the method comprising: receiving sensor data from a sensor during a time period to monitor a characteristic of a blood pressure wave signal in the sensor data during the time period, wherein a cuff is worn by the patient during the time period; inflating at least one bladder of the cuff with a gas until the characteristic of the blood pressure wave signal crosses a characteristic threshold from a first side of the characteristic threshold to a second side of the characteristic threshold at a first time within the time period, wherein the at least one bladder is at a first pressure at the first time; deflating the at least one bladder of the cuff until the characteristic of the blood pressure wave signal crosses the characteristic threshold from the second side of the characteristic threshold to the first side of the characteristic threshold at a second time within the time period, wherein the at least one bladder is at a second pressure at the second time; and identifying a blood pressure value corresponding to the patient, wherein the blood pressure value is between the first pressure and the second pressure.
Aspect 32. The non-transitory computer-readable medium of Aspect 31, wherein the program is executable by the processor to perform operations according to any one of Aspects 2 to 26, or Aspects 28 to 30.
Aspect 33. An apparatus comprising one or more means for performing operations according to any of Aspects 1 to 32.

What is claimed is:

1. A method of blood pressure analysis for a patient, the method comprising:
receiving sensor data from a sensor during a time period to monitor a characteristic of a blood pressure wave signal in the sensor data during the time period, wherein a cuff is worn by the patient during the time period;
inflating at least one bladder of the cuff with a gas until the characteristic of the blood pressure wave signal crosses a characteristic threshold from a first side of the characteristic threshold to a second side of the characteristic threshold at a first time within the time period, wherein the at least one bladder is at a first pressure at the first time;
deflating the at least one bladder of the cuff until the characteristic of the blood pressure wave signal crosses the characteristic threshold from the second side of the characteristic threshold to the first side of the characteristic threshold at a second time within the time period, wherein the at least one bladder is at a second pressure at the second time; and
identifying a blood pressure value corresponding to the patient, wherein the blood pressure value is between the first pressure and the second pressure.

2. The method of claim 1, wherein the blood pressure value is a diastolic blood pressure, wherein the blood pressure wave signal is less than the characteristic threshold on the first side of the characteristic threshold, and wherein the blood pressure wave signal is greater than the characteristic threshold on the second side of the characteristic threshold.

3. The method of claim 1, wherein the blood pressure value is a systolic blood pressure, wherein the blood pressure wave signal is greater than the characteristic threshold on the first side of the characteristic threshold, and wherein the blood pressure wave signal is less than the characteristic threshold on the second side of the characteristic threshold.

4. The method of claim 1, wherein the sensor is a microphone, wherein the sensor data includes audio data recorded by the microphone, wherein the blood pressure wave signal is a Korotkoff audio signal within the audio data, and wherein the characteristic of the blood pressure wave signal is an amplitude of the Korotkoff audio signal.

5. The method of claim 1, wherein the sensor is an electrical attribute sensor, wherein the sensor data includes electrical attribute data measured by the electrical attribute sensor, and wherein the blood pressure wave signal is an impedance signal based on the electrical attribute data.

6. The method of claim 1, wherein inflating at least one bladder of the cuff with the gas includes opening a valve to release an amount of the gas from a pressurized gas canister and to convey the amount of the gas from the pressurized gas canister to the at least one bladder of the cuff.

7. The method of claim 1, wherein deflating the at least one bladder of the cuff includes opening a valve to release an amount of the gas from the at least one bladder of the cuff.

8. The method of claim 1, wherein the cuff includes a plurality of bladders, wherein inflating the at least one bladder of the cuff with the gas includes inflating at least a subset of the plurality of bladders with the gas, and wherein deflating the at least one bladder of the cuff includes deflating at least the subset of the plurality of bladders.

9. The method of claim 1, wherein the at least one bladder includes a first bladder and a second bladder, wherein inflating the at least one bladder of the cuff with the gas includes inflating the first bladder and the second bladder at a shared pressure.

10. The method of claim 1, wherein the at least one bladder includes a first bladder and a second bladder, wherein inflating the at least one bladder of the cuff with the gas includes inflating the first bladder at a first pressure and inflating the second bladder at a second pressure.

11. The method of claim 10, wherein the first bladder is closer to a heart of the patient than the second bladder, and wherein the first pressure is lower than the second pressure.

12. The method of claim 1, wherein the at least one bladder includes a first pillow and a second pillow, wherein inflating the at least one bladder of the cuff with the gas includes inflating the first pillow at a first inflation rate and inflating the second pillow at a second inflation rate, and wherein deflating the at least one bladder of the cuff with the gas includes deflating the first pillow at a first deflation rate and deflating the second pillow at a second deflation rate, wherein the cuff includes overlapping portions that overlap over each other while the cuff is worn by the patient during the time period, wherein one of the overlapping portions includes the first pillow, and wherein a non-overlapping portion of the cuff includes the second pillow.

13. The method of claim 1, further comprising:
receive secondary sensor data from a second sensor during the time period to monitor a secondary blood pressure wave signal in the secondary sensor data during the time period;
inflate a second bladder of the cuff with the gas until the characteristic of the secondary blood pressure wave signal crosses a secondary threshold from a first side of the secondary threshold to a second side of the secondary threshold at a third time within the time period, wherein the second bladder is at a third pressure at the first time;
deflate the second bladder of the cuff until the characteristic of the secondary blood pressure wave signal crosses the secondary threshold from the second side of the secondary threshold to the first side of the secondary threshold at a fourth time within the time period, wherein the second bladder is at a fourth pressure at the fourth time; and
identify a second blood pressure value corresponding to the patient, wherein the second blood pressure value is between the third pressure and the fourth pressure.

14. The method of claim 13, wherein the blood pressure value is a diastolic blood pressure, and wherein the second blood pressure value is a systolic blood pressure.

15. The method of claim 13, wherein the blood pressure value is a systolic blood pressure, and wherein the second blood pressure value is a diastolic blood pressure.

16. The method of claim 1, further comprising:
identifying that a first fastener is in use to secure the cuff to a body part of the patient during the time period, wherein the first fastener is one of a plurality of fasteners of the cuff; and
identifying a size of the body part of the patient based the first fastener being in use to secure the cuff to the body part of the patient during the time period.

17. The method of claim 16, further comprising:
setting the characteristic threshold based the size of the body part of the patient.

18. The method of claim 16, wherein the cuff includes at least one electromagnet, wherein the first fastener includes a ferromagnetic material that is magnetically coupled to the at least one electromagnet of the cuff to secure the cuff to the body part of the patient during the time period, and wherein identifying that the first fastener is in use to secure the cuff to the body part of the patient during the time period is based on detection of an electrical attribute corresponding to the electromagnet in the ferromagnetic material of the first fastener.

19. The method of claim 16, wherein plurality of fasteners include a plurality of electromagnets that each have different electrical attributes, wherein the first fastener includes a first electromagnet of the plurality of electromagnets, and wherein identifying that the first fastener is in use to secure the cuff to the body part of the patient during the time period is based on detection of an electrical attribute corresponding to the first electromagnet.

20. The method of claim 1, further comprising:
inflating a second bladder of the cuff with the gas; and
determining, based on secondary sensor data from a second sensor that is coupled to the second bladder, and before inflating the at least one bladder, that a movement level of the patient has remained below a movement level threshold for a predetermined amount of time.

21. The method of claim 1, further comprising:
calibrating the sensor using a simulated body part, wherein the simulated body part includes a liquid pumped through a channel at a predetermined pressure, and wherein calibrating the sensor includes matching a pressure measurement from the sensor to the predetermined pressure.

22. The method of claim 1, wherein the sensor identifies that the at least one bladder is at the first pressure at the first time, and wherein the sensor identifies that the at least one bladder is at the second pressure at the second time.

23. The method of claim 1, wherein a second sensor identifies that the at least one bladder is at the first pressure at the first time, and wherein the second sensor identifies that the at least one bladder is at the second pressure at the second time.

24. The method of claim 1, wherein identifying the blood pressure value corresponding to the patient is based on a binary search.

25. The method of claim 1, wherein the sensor captures serial measurements until it the serial measurements asymptotically converge, wherein the serial measurements include at least at least one of the sensor data, the first pressure, or the second pressure.

26. The method of claim 1, further comprising:
identifying a physiological attribute based on at least one of the sensor data, the first pressure, or the second pressure, wherein the physiological attribute includes at least one of a central venous pressure, a heart rate, a respiratory rate, or a cardiac output.

27. A system for blood pressure analysis for a patient, the system comprising:
a memory storing instructions; and
a processor that executes the instructions, wherein execution of the instructions by the processor causes the processor to:
receive sensor data from a sensor during a time period to monitor a characteristic of a blood pressure wave signal in the sensor data during the time period, wherein a cuff is worn by the patient during the time period;
inflate at least one bladder of the cuff with a gas until the characteristic of the blood pressure wave signal crosses a characteristic threshold from a first side of the characteristic threshold to a second side of the characteristic threshold at a first time within the time period, wherein the at least one bladder is at a first pressure at the first time;
deflate the at least one bladder of the cuff until the characteristic of the blood pressure wave signal crosses the characteristic threshold from the second side of the characteristic threshold to the first side of the characteristic threshold at a second time within the time period, wherein the at least one bladder is at a second pressure at the second time; and
identify a blood pressure value corresponding to the patient, wherein the blood pressure value is between the first pressure and the second pressure.

28. The system of claim 27, further comprising:
at least one valve; and
at least one actuator, wherein, to inflate the at least one bladder of the cuff, the execution of the instructions by the processor causes the processor to open the at least one valve using the at least one actuator to convey the gas into the at least one bladder from a gas source.

29. The system of claim 27, further comprising:
at least one valve; and
at least one actuator, wherein, to deflate the at least one bladder of the cuff, the execution of the instructions by the processor causes the processor to open the at least one valve using the at least one actuator to convey the gas out of the at least one bladder of the cuff.

30. A non-transitory computer readable storage medium having embodied thereon a program, wherein the program is executable by a processor to perform a method of blood pressure analysis for a patient, the method comprising:
receiving sensor data from a sensor during a time period to monitor a characteristic of a blood pressure wave signal in the sensor data during the time period, wherein a cuff is worn by the patient during the time period;
inflating at least one bladder of the cuff with a gas until the characteristic of the blood pressure wave signal crosses a characteristic threshold from a first side of the characteristic threshold to a second side of the characteristic threshold at a first time within the time period, wherein the at least one bladder is at a first pressure at the first time;
deflating the at least one bladder of the cuff until the characteristic of the blood pressure wave signal crosses the characteristic threshold from the second side of the characteristic threshold to the first side of the characteristic threshold at a second time within the time period, wherein the at least one bladder is at a second pressure at the second time; and
identifying a blood pressure value corresponding to the patient, wherein the blood pressure value is between the first pressure and the second pressure.
